SEQUENCE LISTING (50 sequences)

SEQ ID NO: 1 - DNA, 28 bp, Unknown
  Unknown plasmodium species
gacacgcgtagtcaacatggtgaaaccc

SEQ ID NO: 2 - DNA, 42 bp, Unknown
  Unknown plasmodium species
tattgctttttattttttaactagtcctttttttgaaatgga

SEQ ID NO: 3 - RNA, 23 nt, Artificial sequence
  2′-O Me-modified nucleotides (1)..(23): 2′-O Me-modified nucleotides at all positions.
  Phosphorothioate linkages (1)..(2): Phosphorothioate linkages between positions 1 and 2, 2 and 3, 19 and 20, 20 and 21, 21 and 22, and 22 and 23.
  Phosphorothioate linkages (2)..(3): Phosphorothioate linkages between positions 1 and 2, 2 and 3, 19 and 20, 20 and 21, 21 and 22, and 22 and 23.
  Phosphorothioate linkages (19)..(20): Phosphorothioate linkages between positions 1 and 2, 2 and 3, 19 and 20, 20 and 21, 21 and 22, and 22 and 23.
  Phosphorothioate linkages (20)..(21): Phosphorothioate linkages between positions 1 and 2, 2 and 3, 19 and 20, 20 and 21, 21 and 22, and 22 and 23.
  Phosphorothioate linkages (21)..(22): Phosphorothioate linkages between positions 1 and 2, 2 and 3, 19 and 20, 20 and 21, 21 and 22, and 22 and 23.
  Phosphorothioate linkages (22)..(23): Phosphorothioate linkages between positions 1 and 2, 2 and 3, 19 and 20, 20 and 21, 21 and 22, and 22 and 23.
  hydroxyprolinol linkage (23)..(23): The A at position 23 is linked to cholesterol through a hydroxyprolinol linkage.
acaaacaccauugucacacucca

SEQ ID NO: 4 - RNA, 17 nt, Artificial sequence
  2′-O Me-modified nucleotides (1)..(17): 2′-O Me-modified nucleotides at all positions.
  Phosphorothioate linkages (1)..(2): Phosphorothioate linkages between positions 1 and 2, 2 and 3, 13 and 14, 14 and 15, 15 and 16, and 16 and 17.
  Phosphorothioate linkages (2)..(3): Phosphorothioate linkages between positions 1 and 2, 2 and 3, 13 and 14, 14 and 15, 15 and 16, and 16 and 17.
  Phosphorothioate linkages (13)..(14): Phosphorothioate linkages between positions 1 and 2, 2 and 3, 13 and 14, 14 and 15, 15 and 16, and 16 and 17.
  Phosphorothioate linkages (14)..(15): Phosphorothioate linkages between positions 1 and 2, 2 and 3, 13 and 14, 14 and 15, 15 and 16, and 16 and 17.
  Phosphorothioate linkages (15)..(16): Phosphorothioate linkages between positions 1 and 2, 2 and 3, 13 and 14, 14 and 15, 15 and 16, and 16 and 17.
  Phosphorothioate linkages (16)..(17): Phosphorothioate linkages between positions 1 and 2, 2 and 3, 13 and 14, 14 and 15, 15 and 16, and 16 and 17.
  hydroxyprolinol linkage (17)..(17): The G at position 17 is linked to cholesterol through a hydroxyprolinol linkage.
caccacauaccgcacgg

SEQ ID NO: 5 - DNA, 22 bp, Mouse
gtggtttcagcaattatcgtgg

SEQ ID NO: 6 - DNA, 21 bp, Mouse
gggtccttcagaaacagagac

SEQ ID NO: 7 - DNA, 22 bp, Mouse
gcgatgatgaaccaggttatga

SEQ ID NO: 8 - DNA, 24 bp, Mouse
atctcgagcaagtctttcagtcct

SEQ ID NO: 9 - DNA, 26 bp, Mouse
ttcgggaactatgtggagtcactttg

SEQ ID NO: 10 - DNA, 25 bp, Mouse
cgcaaggctgccctcaaaccctcag

SEQ ID NO: 11 - DNA, 24 bp, Mouse
ccatcaaacgccattatcacacta

SEQ ID NO: 12 - DNA, 25 bp, Mouse
cacacaatggagaactctagcacaa

SEQ ID NO: 13 - DNA, 21 bp, Mouse
gggtgatcctcttccacgaga

SEQ ID NO: 14 - DNA, 20 bp, Mouse
aggggcaccacacccttatc

SEQ ID NO: 15 - DNA, 21 bp, Mouse
gggcgcaatgtcgagaacatg

SEQ ID NO: 16 - DNA, 21 bp, Mouse
ctggcaggacctccatcattc

SEQ ID NO: 17 - DNA, 20 bp, Mouse
agacaagaaccccaacatcc

SEQ ID NO: 18 - DNA, 21 bp, Mouse
caaaggtgtcaaatgggaagg

SEQ ID NO: 19 - DNA, 22 bp, Mouse
caagttttgcgatgtgagactg

SEQ ID NO: 20 - DNA, 23 bp, mouse
ccgtctccagagtaatgttcttg

SEQ ID NO: 21 - DNA, 21 bp, Mouse
ctctgtgggtaccttgatgcc

SEQ ID NO: 22 - DNA, 20 bp, Mouse
ggaagaccctgaactctgcc

SEQ ID NO: 23 - DNA, 20 bp, Mouse
caaatcctgggtgtcaaagc

SEQ ID NO: 24 - DNA, 20 bp, Mouse
catgggcttcagactggtac

SEQ ID NO: 25 - DNA, 22 bp, Mouse
tccgagcagagatcttcaggaa

SEQ ID NO: 26 - DNA, 22 bp, Mouse
tgcaaccaccactcattctgag

SEQ ID NO: 27 - DNA, 21 bp, Mouse
gtgaagaggaagacagcacag

SEQ ID NO: 28 - DNA, 21 bp, Mouse
gcccattccaaccattactcc

SEQ ID NO: 29 - DNA, 22 bp, Mouse
catggttcctaagggatgagag

SEQ ID NO: 30 - DNA, 22 bp, Mouse
catggttcctaagggatgagag

SEQ ID NO: 31 - DNA, 21 bp, Mouse
ccccagtgtccttacagagtg

SEQ ID NO: 32 - DNA, 19 bp, Mouse
gtgcccagagtggatgtct

SEQ ID NO: 33 - DNA, 20 bp, Mouse
cataaagggtcatcgtggct

SEQ ID NO: 34 - DNA, 19 bp, Mouse
ttgagtccgtctttgccag

SEQ ID NO: 35 - DNA, 21 bp, Mouse
gaagtctctgaagctgatggg

SEQ ID NO: 36 - DNA, 22 bp, Mouse
ttgccttgcgtgtttgatattc

SEQ ID NO: 37 - DNA, 25 bp, Mouse
tcacttctactcttgctatctttcg

SEQ ID NO: 38 - DNA, 20 bp, Mouse
cccagaatcccaaccacaag

SEQ ID NO: 39 - DNA, 21 bp, Mouse
ctgtagcccacgtcgtagcaa

SEQ ID NO: 40 - DNA, 21 bp, Mouse
ctggcaccactagttggttgt

SEQ ID NO: 41 - DNA, 22 bp, Unknown
  Unknown vertebrae
tggagtgtgacaatggtgtttg

SEQ ID NO: 42 - DNA, 22 bp, Unknown
  Unknown vertebrae
tggcagtgtcttagctggttgt

SEQ ID NO: 43 - DNA, 23 bp, Unknown
  Unknown vertebrae
taaggtgcatctagtgcagatag

SEQ ID NO: 44 - DNA, 22 bp, Unknown
  Unknown vertebrae
tagcttatcagactgatgttga

SEQ ID NO: 45 - DNA, 21 bp, Unknown
  Unknown vertebrae
cattattacttttggtacgcg

SEQ ID NO: 46 - DNA, 23 bp, Unknown
  Unknown vertebrae
caaagtgctgttcgtgcaggtag

SEQ ID NO: 47 - DNA, 22 bp, Homo sapiens
cgcaaggatgacacgcaaattc

SEQ ID NO: 48 - DNA, 22 bp, Caenorhabditis elegans
tcaccgggtgtaaatcagcttg

SEQ ID NO: 49 - RNA, 23 nt, Artificial sequence
  2′-O Me-modified nucleotides (1)..(23): 2′-O Me-modified nucleotides at all positions.
  Phosphorothioate linkages (1)..(2): Phosphorothioate linkages between positions 1 and 2, 2 and 3, 19 and 20, 20 and 21, 21 and 22, and 22 and 23.
  Phosphorothioate linkages (2)..(3): Phosphorothioate linkages between positions 1 and 2, 2 and 3, 19 and 20, 20 and 21, 21 and 22, and 22 and 23.
  Phosphorothioate linkages (19)..(20): Phosphorothioate linkages between positions 1 and 2, 2 and 3, 19 and 20, 20 and 21, 21 and 22, and 22 and 23.
  Phosphorothioate linkages (20)..(21): Phosphorothioate linkages between positions 1 and 2, 2 and 3, 19 and 20, 20 and 21, 21 and 22, and 22 and 23.
  Phosphorothioate linkages (21)..(22): Phosphorothioate linkages between positions 1 and 2, 2 and 3, 19 and 20, 20 and 21, 21 and 22, and 22 and 23.
  Phosphorothioate linkages (22)..(23): Phosphorothioate linkages between positions 1 and 2, 2 and 3, 19 and 20, 20 and 21, 21 and 22, and 22 and 23.
  hydroxyprolinol linkage (23)..(23): The A at position 23 is linked to cholesterol through a hydroxyprolinol linkage.
acaaacaccauugucacacucca

SEQ ID NO: 50 - RNA, 17 nt, Artificial sequence
  2′-O Me-modified nucleotides (1)..(17): 2′-O Me-modified nucleotides at all positions.
  Phosphorothioate linkages (1)..(2): Phosphorothioate linkages between positions 1 and 2, 2 and 3, 13 and 14, 14 and 15, 15 and 16, and 16 and 17.
  Phosphorothioate linkages (2)..(3): Phosphorothioate linkages between positions 1 and 2, 2 and 3, 13 and 14, 14 and 15, 15 and 16, and 16 and 17.
  Phosphorothioate linkages (13)..(14): Phosphorothioate linkages between positions 1 and 2, 2 and 3, 13 and 14, 14 and 15, 15 and 16, and 16 and 17.
  Phosphorothioate linkages (14)..(15): Phosphorothioate linkages between positions 1 and 2, 2 and 3, 13 and 14, 14 and 15, 15 and 16, and 16 and 17.
  Phosphorothioate linkages (15)..(16): Phosphorothioate linkages between positions 1 and 2, 2 and 3, 13 and 14, 14 and 15, 15 and 16, and 16 and 17.
  Phosphorothioate linkages (16)..(17): Phosphorothioate linkages between positions 1 and 2, 2 and 3, 13 and 14, 14 and 15, 15 and 16, and 16 and 17.
  hydroxyprolinol linkage (17)..(17): The G at position 17 is linked to cholesterol through a hydroxyprolinol linkage.
caccacauaccgcacgg

(19) United States
(12) Patent Application Publication
(10) Pub. No.: US 2025/0339445 A1
(43) Pub. Date: Nov. 6, 2025

(54) Modulators of Orphan Nuclear Receptors for Treating Pancreatitis, Glioblastoma, Sarcopenia and Stroke

(71) Applicant: Emory University, Atlanta, GA (US)

(72) Inventors: Raymond F SCHINAZI, Miami, FL (US); Bryan Cox, Atlanta, GA (US); Eithan Galun, Jerusalem (IL); Franck Amblard, Atlanta, GA (US); Dharmeshkumar Patel, Atlanta, GA (US)

(21) Appl. No.: 18/034,520

(22) PCT Filed: Nov. 1, 2021
(86) PCT No.: PCT/US2021/057628

§ 371 Date: Apr. 28, 2023

Related U.S. Application Data
(60) Provisional application No. 63/108,054, filed on Oct. 30, 2020.

(51) Int. Cl. A61K 31/5513; A61K 31/675; A61K 45/06; A61P 1/18

(52) U.S. Cl. CPC A61K 31/5513; A61K 31/675; A61K 45/06; A61P 1/18

(57) ABSTRACT

Compounds, compositions and methods for modulating retinoic acid receptor-like orphan receptors (ROR) so as to increase FGF21 levels, and treating and preventing disorders associated with FGF21, such as pancreatitis, sarcopenia, stroke, and traumatic brain injury, and to increase miR-122 levels, and treating and preventing disorders such as glioblastoma.

### CROSS-REFERENCE TO RELATED APPLICATIONS

This application claims the benefit of priority under Article 8 PCT of U.S. Provisional Patent Application No. 63/108,054 filed Oct. 30, 2020 and entitled “MODULATORS OF ORPHAN NUCLEAR RECEPTORS FOR USE IN TREATING PANCREATITIS, GLIOBLASTOMA, SARCOPENIA, STROKE, AND TRAUMATIC BRAIN INJURY.” The contents of the above application is incorporated by reference as if fully set forth herein in its entirety.

### FIELD OF THE INVENTION

This application is directed to small molecule modulators of retinoic acid receptor-related orphan receptors (ROR) such as RORα, RORβ, or RORγ for use in treating disorders such as pancreatitis, sarcopenia, stroke, glioblastoma, and traumatic brain injury, which are associated with FGF21 and/or miR122.

### BACKGROUND OF THE INVENTION

Pancreatitis is one of the most common and debilitating diseases of the gastrointestinal tract, leading to substantial morbidity and mortality. Pancreatitis results from the premature activation of digestive enzymes in the pancreas itself, which causes tissue damage and inflammation. Common causes of pancreatitis include alcohol abuse and gallstones. About a third of pancreatitis cases in humans are caused by alcohol, which has the highest rates of morbidity. Pancreatitis also occurs in 5 to 10% of patients undergoing endoscopic retrograde cholangiopancreatography (ERCP), a procedure used to examine the pancreatic and biliary ducts as well as the gallbladder.

There are no specific therapies for this severe clinical condition. Treatments for pancreatitis are generally supportive in nature. Thus, there is a pressing need for new therapies.

Pancreatitis initiates from the activation of digestive enzymes in the pancreas, which causes tissue damage and inflammation. Common causes of pancreatitis include alcohol abuse, hyperlipidemia and gallstones movement out of the biliary system. Pancreatitis is also iatrogenic, occurs in 5 to 10% of patients undergoing endoscopic retrograde cholangiopancreatography (ERCP). Collectively, pancreatitis is an unmet therapeutic need.

Pancreatitis is a fibroblast growth factor 21 (FGF21)-deficient state, and can be corrected by increasing FGF21 levels. A discussion of the relationship between pancreatitis and FGF21 is provided below.

Fibroblast growth factor 21 (FGF21) is a hormone secreted by the liver in response to diverse metabolic stresses including starvation and the consumption of alcohol or simple sugars. FGF21 acts on a heteromeric cell surface receptor complex composed of a conventional FGF receptor, FGFR1c, together with an obligate co-receptor, β-klotho (7-9). FGF21 is also highly expressed in the exocrine pancreas, where it acts directly on acinar cells in an autocrine/paracrine manner to stimulate digestive enzyme secretion. This prevents protein overload and relieves endoplasmic reticulum (ER) stress. Mice lacking FGF21 are particularly susceptible to pancreatitis induced by the cholecystokinin (CCK) analog cerulean. Conversely, genetic overexpression of FGF21 confers protection in this model. Likewise, prophylactic FGF21 administration reduces fibrogenesis in a mouse model of 1-arginine-induced chronic pancreatitis.

The exocrine pancreas expresses the highest concentrations of FGF21 in the body, where it maintains acinar cell proteostasis. As has been shown in both mouse and human models, acute and chronic pancreatitis is associated with a loss of FGF21 expression, due to activation of the integrated stress response (ISR) pathway. Mechanistically, activation of the ISR in cultured acinar cells and mouse pancreata induced the expression of ATF3, a transcriptional repressor that directly bound to specific sites on the Fgf21 promoter and resulted in loss of FGF21 expression. These ATF3 binding sites are conserved in the human FGF21 promoter.

Consistent with the mouse studies, the reciprocal expression of ATF3 and FGF21 was observed in the pancreata of human patients with pancreatitis. Pharmacologic replacement of FGF21 mitigated the ISR and resolved pancreatitis. Likewise, inhibition of the ISR with an inhibitor of the PKR-like endoplasmic reticulum kinase (PERK) also restored FGF21 expression and alleviated pancreatitis. These findings highlight the importance of FGF21 in preserving exocrine pancreas function.

A positive correlation of serum FGF21 levels and sarcopenia has also been made (See Tezze et al. Age-associated loss of OPA1 in muscle impacts muscle mass, metabolic homeostasis, systemic inflammation, and Epithelial Senescence. Cell Metab 2017; 25:1374-1389 e6).

FGF21 also has effects in treating traumatic brain injury and stroke (see, for example, Jiang et al., “Abstract WMP81: FGF21 Reduces Post-Stroke Blood Brain Barrier Damage in Diabetic db/db Male Mice,” Stroke, Vol 51, Issue Suppl_1 (February 2020)). Jiang discloses that recombinant human Fibroblast growth factor 21 (rFGF21) protects against post-stroke BBB damage by PPARγ activation of the cerebral micovascular endothelium. See also Chen et al., “FGF21 Protects the Blood-Brain Barrier by Upregulating PPARγ via FGFR1/β-klotho after Traumatic Brain Injury,” Journal of Neurotrauma, Vol. 35, No. 17 (2018)).

Blood-brain barrier (BBB) disruption and dysfunction result in brain edema, which is responsible for more than half of all deaths after severe traumatic brain injury (TBI). Chen discloses that fibroblast growth factor 21 (FGF21) has a potential neuroprotective function in the brain. The effects of recombinant human FGF21 (rhFGF21) on BBB integrity and on tight junction (TJ) and adhesion junction (AJ) proteins were investigated both in a TBI mouse model and an in vitro BBB disruption model established with tumor necrosis factor alpha (TNF-α)-induced human brain microvascular endothelial cells (HBMECs). The ability of rhFGF21 to form an FGF21/FGFR1/β-klotho complex was confirmed by in vitro 0-klotho small interfering RNA (siRNA) transfection and FGFR1 co-immunoprecipitation. rhFGF21 markedly reduced neurofunctional behavior deficits and cerebral edema degree, preserved BBB integrity, and recued brain tissue loss and neuron apoptosis in the mouse model after TBI. Both in vivo and in vitro, rhFGF21 upregulated TJ and AJ proteins, thereby preserving the BBB. Moreover, rhFGF21 activated PPARγ in TNF-α-induced HBMECs through formation of an FGF21/FGFR1/β-klotho complex. rhFGF21 protected the BBB through FGF21/FGFR1/β-klotho complex formation and PPARγ activation, which upregulated TJ and AJ proteins. Accordingly, FGF21 is useful for treating traumatic brain injury and other disorders caused by BBB disruption, brain abscesses, De Vivo disease, HIV encephalitis, meningitis, multiple sclerosis, and neuromyelitis optica.

FGF21 is administered by injection, so for reasons of patient compliance, it would be advantageous to provide compounds that can be orally administered to treat or prevent pancreatitis, sarcopenia, stroke, glioblastoma, or traumatic brain injury, or to reduce the susceptibility to, reduce the severity, or delay the progression of these disorders. The present invention provides such compounds, and methods for using the compounds.

### SUMMARY OF THE INVENTION

In one embodiment, RORα agonist compounds, compositions including these compounds, and methods for treating or preventing pancreatitis, sarcopenia, stroke, glioblastoma, traumatic brain injury, or reducing the susceptibility to, reducing the severity of, or delaying the progression of these disorders, are disclosed. In other embodiments, the compounds are used for other disorders associated with FGF21 deficiency, or which can benefit from greater than normal FGF21 levels and/or miR122.

Fibroblast growth factor 21 (FGF21) is a hormone secreted by the liver in response to diverse metabolic stresses. FGF21 is expressed in the exocrine pancreas, to stimulate digestive enzyme secretion. FGF21 knockout (KO) mice are particularly susceptible to pancreatitis. Overexpression of FGF21 confers protection from pancreatitis. Prophylactic FGF21 administration reduces fibrogenesis in a mouse model of pancreatitis. Loss of FGF21 is a driving factor of pancreatitis. Using FGF21 therapeutically reverses preexisting pancreatitis.

Since the RORα agonists described herein increase expression of endogenous FGF21, the RORα agonists can be used to treat, prevent, reduce the susceptibility to, reduce the severity of, or delay the progression of pancreatitis.

In some aspects of this embodiment, methods are provided for modulating the bioactivity of ROR in a subject in a way that increases the subject's endogenous FGF21 levels. Increasing the FGF21 levels treats, prevents, reduces the susceptibility to, reduces the severity of, or delays the progression of disorders associated with FGF21 deficiency, such as pancreatitis or sarcopenia, and also provides a neuroprotective effect to help patients with stroke, traumatic brain injury, and the like.

In other aspects of this embodiment, methods are provided for modulating the bioactivity of ROR in a subject in a way that increases the subject's endogenous miR122 levels. Increasing the miR122 levels treats, prevents, reduces the susceptibility to, reduces the severity of, or delays the progression of disorders associated with miR122, such as those involving lipid droplet formation, such as glioblastoma and the like.

The methods involve contacting the ROR with an effective amount of a compound of formula (A) as shown below, wherein the compound is an agonist or an activator of RORA (also referred to herein as RORα).

In another embodiment, the compound has the following formula:

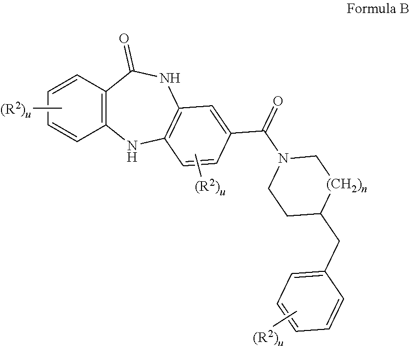

and pharmaceutically-acceptable salts and prodrugs thereof, wherein R² and u are as elsewhere herein with respect to Formula A.

One representative compound has the following formula:

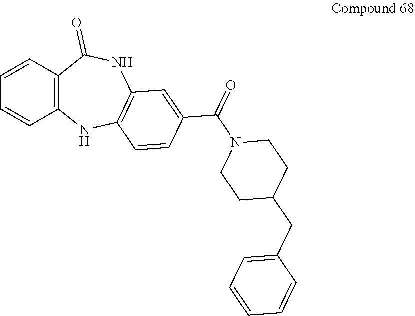 [SMILES: O=C1Nc2cc(C(=O)N3CCC(Cc4ccccc4)CC3)ccc2Nc2ccccc21]

and pharmaceutically-acceptable salts and prodrugs thereof, where R² and u are defined as described elsewhere herein for Formula A.

In another embodiment, the compounds are benzodiazepines that bind with relatively high affinity to the RORA receptor, are agonists of the RORA receptor, and do not cross the blood brain barrier and/or do not bind with a high affinity to GABA receptors, such as the GABA-A receptor. In this embodiment, the compounds can be used to treat a variety of disorders, including pancreatitis and sarcopenia, which are associated with FGF21.

In yet another embodiment, the compounds are benzodiazepines that bind with relatively high affinity to the RORA receptor, are agonists of the RORA receptor, and do cross the blood brain barrier, but do not bind with a high affinity to GABA receptors, such as the GABA-A receptor. In this embodiment, the compounds can be used to treat a variety of neurological disorders, including stroke, and traumatic brain injury, which are associated with FGF21.

In some embodiments, the benzodiazepines are instead used to treat fatty liver disease, such as NASH, as well as cirrhosis of the liver caused by progression of fatty liver disease.

The compounds described herein can be in the form of stereoisomers, polymorphs, salt forms and prodrug forms.

In various embodiments, pharmaceutical compositions and formulations with an effective compound of Formula (A)-(H) are provided to treat, prevent, reduce the susceptibility to, reduce the severity of, or delay the progression of conditions associated with FGF21 deficiency, such as pancreatitis, sarcopenia, stroke, and traumatic brain injury. The compositions can include a compound of Formula (A)-(H), and a pharmaceutically-acceptable carrier or excipient, and can optionally comprise one or more additional active agents.

In various embodiments, pharmaceutical compositions and formulations with an effective compound of Formulas (B)-(H) are provided to treat, prevent, reduce the susceptibility to, reduce the severity of, or delay the progression of conditions associated with FGF21 deficiency, such as pancreatitis, sarcopenia, stroke, and traumatic brain injury. The compositions can include compound of Formulas (B)-(H) and a pharmaceutically-acceptable carrier or excipient, and can optionally comprise one or more additional active agents. Specifically listed R¹ variables for Formula A can also be used with any of Formulas (B)-(H).

The present invention will be better understood with reference to the following Detailed Description.

### BRIEF DESCRIPTION OF THE DRAWINGS

FIG. 1 is a chart showing how Compound 1 induces expression of RORα-regulated luciferase with a WT RORE but has no activity when the RORE is mutated. Data are presented in terms of relative lucerifase activity versus concentration, with error bars=SD. *P<0.05 compared to DMSO.

FIG. 2 is a chart showing how Compound 1 specifically increases secretion of miR-122 from Huh-7 cells, shown in terms of relative microRNA levels in Huh7 medium versus DMSO control and Compound 1 (1 μM). Data are presented as error bars=SD. **P<0.01.

FIGS. 3A-B are charts showing how Compound 1 modulates Th17 populations in human peripheral mononuclear cells (PBMCs). FIG. 3A shows how the viability of CD4⁺ T cells was determined by LIVE/DEAD fixable aqua dead cell staining, shown as % viability over the total CD4+ Th17 cell population. FIG. 3B shows the total percent composition of CD4⁺ Th17 cells (in terms of % Th17 cells) as determined by gating on CD3⁺/CD4⁺/CD45RA⁻/CXCR3⁻/CCR4⁺CXCR5⁻/CCR6⁺ cells. These results show that Compound 1 decreases the CD4⁺ Th17 population selectively under stimulating conditions.

FIGS. 4A-E are charts showing how Compound 1 increases expression of RORα target genes in mice (n=3)—miR-122 and Gpase6. FIG. 4A shows plasma levels, and FIG. 4B shows liver levels, of miR-122 levels measured over 7 days. FIG. 4C shows mRNA levels of miR-122 and RORα target genes (Aldoa and Gpase6, respectively), and miR-122 precursor were measured over 7 days. The data show that secreted miR-122 enters periphery tissues. FIGS. 4D and 4E show miR-122 levels in skeletal muscle (4D) and white adipose tissues (“WAT”) (4E) were measured over 7 days. The data show that miR-18 and miR-126 were not affected following treatment with Compound 1. Data are presented as error bars=SD. *P<0.05, **P<0.01, ***P<0.001 compare to saline. White bars are control (saline), red bars are results from Day 1, pink bars are results from Day 3, and purple bars are results from Day 7.

FIGS. 5A-C are charts showing Compound 1 (Cmpd1) treatment reduces body weight and increases energy expenditure via miR-122 activity in high-fat-fed C57BL/6 mice. FIG. 5 A shows the change in body weight (grams) before (blue) and after (red) 3 weeks of treatment. FIG. 5B shows the qRT-PCR analysis of relative miR-122 levels in plasma at the final time point. FIG. 5C is a chart showing the colorimetric quantification of b-hydroxybutyrate plasma levels (in nM) 3 weeks after treatment. FIG. 5D is a photograph of a representative lipid accumulation visualized by H&E staining of liver sections. N=5. Data are presented as error bars=SD. *P<0.05, **P<0.01, ***P<0.001.

FIGS. 6A-B are charts showing that Compound 1 administration increases miR-122 expression and reduces liver and muscle triglyceride levels in Sgp130FC mice (n=3). Sgp130FC mice were injected (ip) with Compound 1 for four weeks (7.5 mg/kg, twice/week). FIGS. 6A-6B show the qRT-PCR analysis of miR-122 levels in plasma (6A), and in the livers (6B) after 4 weeks treatment with saline (as a control) and Compound 1. MicroRNA-18 was used as a negative control and its plasma and liver levels were not affected following treatment with Compound 1. The effect seen on this microRNA in FIG. 6A is not significant compared to the significant effect seen on miR-122.

FIGS. 7A and B show the results of qRT-PCR analysis of miR-122 extracted from plasma and liver, respectively, in mice treated with Compound 1 or saline. FIG. 7C shows the qRT-PCR analysis of FGF21 and G6pc, as well as RORα target genes, pri- and pre-miR-122 mRNA, extracted from mice livers. FIG. 7D is a chart showing the quantification of liver triglyceride (TG) levels (mg/dL) for mice administered saline or Compound 1.

FIGS. 8A-D are charts showing various markers of liver damage in C57BL/6 mice fed for 3 weeks with an atherogenic diet (to induce fibrosis) and injected with 15 mg/kg Compound 1 (or saline+DMSO) 3 times a week for 3.5 weeks (n=8). qRT-PCR analysis of miR-122 extracted from plasma (FIG. 8A) and liver (FIG. 8B) for the untreated (grey bars) and treated (black bars) cohorts. miR-93 and miR-18 were included for negative controls in plasma and liver, respectively. FIG. 8C is a chart that shows ALT and AST plasma levels measured at the end of the experiment. FIG. 8D is a chart showing qRT-PCR analysis of mRNA of genes involved in fibrosis and RORα target gene (FGF21) extracted from mice livers. microRNA levels in the plasma were normalized to spiked C. elegans miR-39; microRNA levels in the tissues were normalized to RNU6. mRNA levels were normalized to HPRT. Data are presented as error bars=SD. *P<0.05, **P<0.01. ***P<0.001, ****P<0.0001.

FIG. 9A shows representative microphotographs of H&E, CD3 and F4/80-stained livers taken from saline or Compound 1-treated mice; scale bars represent 10 μm. FIG. 9B is a chart showing quantification of positively-stained F4/80 areas using ImageJ.

FIGS. 10A and 10C are microphotographs of Masson Trichrome (M.T.) and α-SMA stained livers taken from saline or Compound 1-treated mice; scale bars represent 10 μm. FIGS. 10B and D are graphs showing quantification of positively-stained areas using ImageJ (%). M.T. staining is shown in FIG. 10B and SMA staining is shown in FIG. 10D.

FIG. 11 shows expression levels of RORα, of RORα and MIR122 target genes, in livers of NASH patients. Database-based gene expression analysis conducted using human public datasets obtained from the NCBI GEO database (http://www.ncbi.nlm.nih.gov/geo/), processed as median-normalized signal intensity values measuring liver mRNA levels in normal and steatohepatitis patients. A positive correlation between pre-MIR122 and RORα target gene (FGF21) expression levels was shown in the livers of patients with NASH. The positive correlation coefficient (r2) was calculated by Pearson correlation test. n=25 (normal) and n=14 (NASH) for GEO: GSE89632.

FIGS. 12-13 show that the RORα agonist Compound 1 reduced steatosis via increased MIR122 expression in HFD-fed mice. The figures show levels of MIR122 (Agpat1 and Dgat1) and RORα (FGF21) target genes in the liver and muscle. FIG. 13 shows an RNA-seq analysis of RNA extracted from liver tissues showing a positive correlation between pri-MIR122 and FGF21 (RORα target gene) mRNA expression. microRNA levels in the plasma were normalized to spiked C. elegans miR-39; microRNA levels in the tissues were normalized to RNU6. mRNA levels were normalized to HPRT. Data are represented as mean±SD. N=6. *P<0.05, **P<0.01. ***P<0.001, ****P<0.0001.

FIG. 14 shows the results of a qRT-PCR analysis of RORα target genes and pri- and pre-MIR122 mRNA extracted from mice livers. The data show that the RORα agonist, Compound 1, increased levels of MIR122.

FIG. 15 shows the anti-inflammatory and anti-fibrogenic effects of Compound 1. A qRT-PCR analysis of mRNA of genes involved in fibrosis and the RORα target gene (FGF21) extracted from mice livers, where microRNA levels in the plasma were normalized to spiked C. elegans miR-39, and microRNA levels in the tissues were normalized to RNU6. mRNA levels were normalized to HPRT. Data are represented as mean±SD. *P<0.05, **P<0.01. ***P<0.001.

FIG. 16 is a chart showing relative FGF21 expression based on various concentrations of SR1078 (μM).

FIG. 17 is a schematic illustration of the ligand binding domain of the RORA receptor, with compounds shown docked inside the domain.

FIGS. 18A and B are schematic illustrations of the binding of a putative agonist of the RORA receptor to the miR-122 promoter, showing how when an agonist binds to the receptor, one can measure lucerifase activity, and when a compound is not an agonist, there is no luciferase activity.

FIG. 19 is a chart showing the relative luceriferase activity against wild type and mutant RORα for various concentrations of Compound 68.

### DETAILED DESCRIPTION

The compounds described herein of Formula (A)-(H) modulate expression of ROR target genes in hepatocyte cells, particularly those related to production of miR-122 and subsequent production of FGF21.

Increased production of FGF21 is useful for treating a variety of disorders, including pancreatitis, sarcopenia, stroke, and traumatic brain injury, which are associated with FGF21.

Increased production of miR-122 also reduces formation of lipid droplets, and glioblastoma (GBM) cells form lipid droplets as a way to avoid lipotoxicity. Accordingly, administration of the compounds described herein to a subject increases the subject's endogenous miR122 levels, which, in turn, treats, prevents, reduces the susceptibility to, reduces the severity of, or delays the progression of disorders associated with miR-122, such as those involving lipid droplet formation, such as glioblastoma (GBM) and the like.

Pharmaceutical formulations including one or more compounds described herein, in combination with a pharmaceutically acceptable carrier or excipient, are also disclosed. In one embodiment, the formulations include at least one compound described herein and at least one further therapeutic agent.

The present invention will be better understood with reference to the following definitions:

### I. Definitions

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term “modulator” includes antagonists, allosteric inhibitors, agonists, and partial agonists. Certain modulators can shut down ROR expression (antagonists and allosteric inhibitors directly, and partial agonists in a dose-dependent manner), and others (agonists and partial agonists, the latter in a dose-dependent manner) can increase ROR expression.

As used herein, the term “enantiomerically pure” refers to a compound composition that comprises at least approximately 95%, and, preferably, approximately 97%, 98%, 99% or 100% of a single enantiomer of that compound.

As used herein, the term “substantially free of” or “substantially in the absence of” refers to a compound composition that includes at least 85 to 90% by weight, preferably 95% to 98% by weight, and, even more preferably, 99% to 100% by weight, of the designated enantiomer of that compound. In a preferred embodiment, the compounds described herein are substantially free of enantiomers.

Similarly, the term “isolated” refers to a compound composition that includes at least 85 to 90% by weight, preferably 95% to 98% by weight and, even more preferably, 99% to 100% by weight, of the compound, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃. Where the alkyl moiety is substituted at both ends, it is an “alkylene” moiety, such as a methylene moiety, and such are intended to be encompassed herein.

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

As used herein, a “bridged alkyl” refers to a bicyclo- or tricycloalkane, for example, a 2:1:1 bicyclohexane.

As used herein, a “spiro alkyl” refers to two rings that are attached at a single (quaternary) carbon atom.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “fatty alcohol” as used herein refers to straight-chain primary alcohols with between 4 and 26 carbons in the chain, preferably between 8 and 26 carbons in the chain, and most preferably, between 10 and 22 carbons in the chain. The precise chain length varies with the source. Representative fatty alcohols include lauryl, stearyl, and oleyl alcohols. They are colorless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. They are widely used in industry. As with fatty acids, they are often referred to generically by the number of carbon atoms in the molecule, such as “a C₁₂ alcohol”, that is an alcohol having 12 carbons, for example dodecanol.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, arylkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from the group consisting of straight, branched, or cyclic alkyl or lower alkyl, alkoxyalkyl, including, but not limited to methoxymethyl, aralkyl, including, but not limited to, benzyl, aryloxyalkyl, such as phenoxymethyl, aryl, including, but not limited to, phenyl, optionally substituted with halogen (F, Cl, Br, or I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituents selected from the group consisting of halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluene sulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism to which the compounds are administered, including but not limited to cell lines and animals, and, preferably, humans. The term host specifically refers to primates (including but not limited to chimpanzees) and humans. In most animal applications of the present invention, the host is a human being. Veterinary applications, in certain indications, however, are clearly contemplated by the present invention (such as for use in treating chimpanzees).

The term “peptide” refers to a natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester) compound which, upon administration to a patient, provides the compound. Pharmaceutically-acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

Pharmaceutically acceptable prodrugs refer to a compound that is metabolized, for example hydrolyzed or oxidized, in the host to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds of this invention can possess antiviral activity, can be metabolized to form a compound that exhibits such activity, or both.

Non-limiting examples of phosphate/phosponate prodrugs are described in the following references: Ho, D. H. W. (1973) “Distribution of Kinase and deaminase of 1-beta-D-arabinofuranosylcytosine in tissues of man and muse.” Cancer Res. 33, 2816-2820; Holy, A. (1993) Isopolar phosphorous-modified nucleotide analogues,” In: De Clercq (Ed.), Advances in Antiviral Drug Design, Vol. I, JAI Press, pp. 179-231; Hong, C. I., Nechaev, A., and West, C. R. (1979a) “Synthesis and antitumor activity of 1-beta-D-arabino-furanosylcytosine conjugates of cortisol and cortisone.” Bicohem. Biophys. Rs. Commun. 88, 1223-1229; Hong, C. I., Nechaev, A., Kirisits, A. J. Buchheit, D. J. and West, C. R. (1980) “Nucleoside conjugates as potential antitumor agents. 3. Synthesis and antitumor activity of 1-(beta-D-arabinofuranosyl)cytosine conjugates of corticosteroids and selected lipophilic alcohols.” J. Med. Chem. 28, 171-177; Hosteller, K. Y., Stuhmiller, L. M., Lenting, H. B. M. van den Bosch, H. and Richman J. Biol. Chem. 265, 6112-6117; Hosteller, K. Y., Carson, D. A. and Richman, D. D. (1991); “Phosphatidylazidothymidine: mechanism of antiretroviral action in CEM cells.” J. Biol Chem. 266, 11714-11717; Hosteller, K. Y., Korba, B. Sridhar, C., Gardener, M. (1994a) “Antiviral activity of phosphatidyl-dideoxycytidine in hepatitis B-infected cells and enhanced hepatic uptake in mice.” Antiviral Res. 24, 59-67; Hosteller, K. Y., Richman, D. D., Sridhar. C. N. Felgner, P. L. Felgner, J., Ricci, J., Gardener, M. F. Selleseth, D. W. and Ellis, M. N. (1994b) “Phosphatidylazidothymidine and phosphatidyl-ddC: Assessment of uptake in mouse lymphoid tissues and antiviral activities in human immunodeficiency virus-infected cells and in rauscher leukemia virus-infected mice.” Antimicrobial Agents Chemother. 38, 2792-2797; Hunston, R. N., Jones, A. A. McGuigan, C., Walker, R. T., Balzarini, J., and DeClercq, E. (1984) “Synthesis and biological properties of some cyclic phosphotriesters derived from 2′-deoxy-5-flourouridine.” J. Med. Chem. 27, 440-444; Ji, Y. H., Moog, C., Schmitt, G., Bischoff, P. and Luu, B. (1990); “Monophosphoric acid esters of 7-.beta.-hydroxycholesterol and of pyrimidine nucleoside as potential antitumor agents: synthesis and preliminary evaluation of antitumor activity.” J. Med. Chem. 33 2264-2270; Jones, A. S., McGuigan, C., Walker, R. T., Balzarini, J. and DeClercq, E. (1984) “Synthesis, properties, and biological activity of some nucleoside cyclic phosphoramidates.” J. Chem. Soc. Perkin Trans. I, 1471-1474; Juodka, B. A. and Smrt, J. (1974) “Synthesis of diribonucleoside phosph (P.fwdarw.N) amino acid derivatives.” Coll. Czech. Chem. Comm. 39, 363-968; Kataoka, S., Imai, J., Yamaji, N., Kato, M., Saito, M., Kawada, T. and Imai, S. (1989) “Alkylated cAMP derivatives; selective synthesis and biological activities.” Nucleic Acids Res. Sym. Ser. 21, 1-2; Kataoka, S., Uchida, “(cAMP) benzyl and methyl triesters.” Heterocycles 32, 1351-1356; Kinchington, D., Harvey, J. J., O'Connor, T. J., Jones, B. C. N. M., Devine, K. G., Taylor-Robinson D., Jeffries, D. J. and McGuigan, C. (1992) “Comparison of antiviral effects of zidovudine phosphoramidate an dphosphorodiamidate derivates against HIV and ULV in vitro.” Antiviral Chem. Chemother. 3, 107-112; Kodama, K., Morozumi, M., Saithoh, K. I., Kuninaka, H., Yosino, H. and Saneyoshi, M. (1989) “Antitumor activity and pharmacology of 1-.beta.-D-arabinofuranosylcytosine-5′-stearylphosphate; an orally active derivative of 1-.beta.-D-arabinofuranosylcytosine.” Jpn. J. Cancer Res. 80, 679-685; Korty, M. and Engels, J. (1979) “The effects of adenosine- and guanosine 3′,5′ phosphoric and acid benzyl esters on guinea-pig ventricular myocardium.” Naunyn-Schmiedeberg's Arch. Pharmacol. 310, 103-111; Kumar, A., Goe, P. L., Jones, A. S. Walker, R. T. Balzarini, J. and DeClercq, E. (1990) “Synthesis and biological evaluation of some cyclic phosphoramidate nucleoside derivatives.” J. Med. Chem, 33, 2368-2375; LeBec, C., and Huynh-Dinh, T. (1991) “Synthesis of lipophilic phosphate triester derivatives of 5-fluorouridine an arabinocytidine as anticancer prodrugs.” Tetrahedron Lett. 32, 6553-6556; Lichtenstein, J., Barner, H. D. and Cohen, S. S. (1960) “The metabolism of exogenously supplied nucleotides by Escherichia coli.,” J. Biol. Chem. 235, 457-465; Lucthy, J., Von Daeniken, A., Friederich, J. Manthey, B., Zweifel, J., Schlatter, C. and Benn, M. H. (1981) “Synthesis and toxicological properties of three naturally occurring cyanoepithioalkanes”. Mitt. Geg. Lebensmittelunters. Hyg. 72, 131-133 (Chem. Abstr. 95, 127093); McGigan, C. Tollerfield, S. M. and Riley, P.a. (1989) “Synthesis and biological evaluation of some phosphate triester derivatives of the antiviral drug Ara.” Nucleic Acids Res. 17, 6065-6075; McGuigan, C., Devine, K. G., O'Connor, T. J., Galpin, S. A., Jeffries, D. J. and Kinchington, D. (1990a) “Synthesis and evaluation of some novel phosphoramidate derivatives of 3′-azido-3′-deoxythymidine (AZT) as anti-HIV compounds.” Antiviral Chem. Chemother. 1 107-113; McGuigan, C., O'Connor, T. J., Nicholls, S. R. Nickson, C. and Kinchington, D. (1990b) “Synthesis and anti-HIV activity of some novel substituted dialkyl phosphate derivatives of AZT and ddCyd.” Antiviral Chem. Chemother. 1, 355-360; McGuigan, C., Nicholls, S. R., O'Connor, T. J., and Kinchington, D. (1990c) “Synthesis of some novel dialkyl phosphate derivative of 3′-modified nucleosides as potential anti-AIDS drugs.” Antiviral Chem. Chemother. 1, 25-33; McGuigan, C., Devin, K. G., O'Connor, T. J., and Kinchington, D. (1991) “Synthesis and anti-HIV activity of some haloalkyl phosphoramidate derivatives of 3′-azido-3′-deoxythylmidine (AZT); potent activity of the trichloroethyl methoxyalaninyl compound.” Antiviral Res. 15, 255-263; McGuigan, C., Pathirana, R. N., Balzarini, J. and DeClercq, E. (1993b) “Intracellular delivery of bioactive AZT nucleotides by aryl phosphate derivatives of AZT.” J. Med. Chem. 36, 1048-1052.

### II. Active Compounds

In one embodiment, the compounds have the following formula:

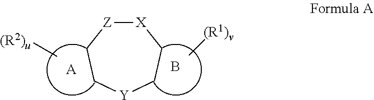

or a pharmaceutically acceptable salt or prodrug thereof.

In this formula:
one of X and Z is selected from the group consisting of —NH—, —N(NH₂)—, —NH(OH)—, N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, —O—, —CH₂—, —CH(C₁₋₁₀ alkyl)-, C(C₁₋₁₀ alkyl)₂-, —CH(C₃₋₁₀ cycloalkyl)-, —CH(C₂₋₁₀ alkenyl, —CH(C₂₋₁₀ alkynyl)-, —CH(aryl)-, —CH(heteroaryl)-, —CF₂—, —CCl₂—, —CH(CF₃)—, —CH(OH)—, —CH(O—C₁₋₁₀ Alkyl)-, —CH(NH₂)—, —CH(NH—C₁₋₁₀ Alkyl)-, and —CH(C(O)NH₂)—,
and the other one of X and Z is selected from the group consisting of —C(O)—, —SO₂—, —N(C(O)—, —CH₂—, —CH(C₁₋₁₀ alkyl)-, C(C₁₋₁₀ alkyl)₂-, —CH(C₃₋₁₀ cycloalkyl)-, —CH(C₂₋₁₀ alkenyl, —CH(C₂₋₁₀ alkynyl)-, —CH(aryl)-, —CH(heteroaryl)-, —CF₂—, —CCl₂—, —CH(CF₃)—, —CH(OH)—, —CH(OAlkyl)-, —CH(NH₂)—, —CH(NHC₁₋₁₀ Alkyl)-, and —CH(C(O)NH₂)—,
Y is selected from the group consisting of —NH, —N(NH₂)—, —NH(OH)—, N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, —O—, —CH₂—, —CH(C₁₋₁₀ alkyl)-, —CH(C₃₋₁₀ cycloalkyl)-, —CH(C₂₋₁₀ alkenyl, —CH(C₂₋₁₀ alkynyl)-, —CH(aryl)-, —CH(heteroaryl)-, —C(C₁₋₁₀ alkyl)₂-, —CF₂—, —CCl₂—, —CH(CF₃)—, —CH(OH)—, —CH(O—C₁₋₁₀ Alkyl)-, —C(O)—, —SO₂—, —N(C(O)—C₁₋₁₀ Alkyl)-, —N(C(O)O—C₁₋₁₀ Alkyl)-, —CH(NH₂)—, —CH(NH—C₁₋₁₀ Alkyl)-, and —CH(C(O)NH₂)—,
A and B are, independently, phenyl, a five-membered heteroaromatic ring containing one, two or three nitrogen, oxygen, or sulfur atoms, or a six-membered heteroaromatic ring containing one, two or three nitrogen atoms;
u and v are independently 0, 1, 2, 3 or 4; with the proviso that at least one of u and v is 1, 2, 3, or 4;
each R¹ and R² are independently R³, OH, OR³, SR³, S(O)R³, SO₂R³, C(O)R³, C(O)OR³, OC(O)R³, OC(O)OR³, NH₂, NHR³, NHC(O)R³, NR³C(O)R³, NHS(O)₂R³, NR³S(O)₂R³, NHC(O)OR³, NR³C(O)OR³, NHC(O)NH₂, NHC(O)NHR³, NHC(O)N(R³)₂, NR³C(O)N(R³)₂, C(O)NH₂, C(O)NHR³, C(O)N(R³)₂, C(O)NHOH, C(O)NHOR³, C(O)NHSO₂R³, C(O)NR³SO₂R³, SO₂NH₂, SO₂NHR³, SO₂N(R³)₂, COOH, C(O)H, C(N)NH₂, C(N)NHR³, C(N)N(R³)₂, C(N)OH, C(N)OCH₃, CN, N₃, NO₂, CF₃, CF₂CF₃, OCF₃, OCF₂CF₃, halo (F, Cl, Br, or I), —CH₂-phosphonate, —CH₂O-phosphate, CH₂P(O)(OH)₂, CH₂P(O)(OR³)₂, CH₂P(O)(OR³)(NR³), CH₂P(O)(NR³)₂, CH₂P(O)(OH)(OC₁₋₁₀ alkyl-O—C₁₋₂₀ alkyl), or CH₂-cycloSal monophosphate prodrug,
wherein the term phosphate includes monophosphate, diphosphate, triphosphate, and stabilized phosphate prodrugs, and the term phosphonate includes the same prodrugs that are present in the phosphate prodrugs,
and when R¹ and R² are on adjacent carbon, they can come together to form an saturated or unsaturated alkyl, an aromatic or a heteroaromatic ring;
each R³ is, independently, aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁴, OH, OR⁴, SR⁴, S(O)R⁴, SO₂R⁴, C(O)R⁴, C(O)OR⁴, OC(O)R⁴, OC(O)OR⁴, NH₂, NHR⁴, NHC(O)R⁴, NR⁴C(O)R⁴, NHS(O)₂R⁴, NR⁴S(O)₂R⁴, NHC(O)OR⁴, NR⁴C(O)OR⁴, NHC(O)NH₂, NHC(O)NHR⁴, NHC(O)N(R⁴)₂, NR⁴C(O)N(R⁴)₂, C(O)NH₂, C(O)NHR⁴, C(O)N(R⁴)₂, C(O)NHOH, C(O)NHOR⁴, C(O)NHSO₂R⁴, C(O)NR⁴SO₂R⁴, SO₂NH₂, SO₂NHR⁴, SO₂N(R⁴)₂, COOH, C(O)H, C(N)NH₂, C(N)NHR⁴, C(N)N(R⁴)₂, C(N)OH, C(N)OCH⁴, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), cycloSal monophosphate prodrugs, CH₂P(O)(OH)₂, CH₂P(O)(OR⁴)₂, CH₂P(O)(OR⁴)(NR⁴), CH₂P(O)(NR⁴)₂, CH₂P(O)(OH)(OC₁₋₁₀ alkyl-O—C₁₋₂₀ alkyl), and CH₂-cycloSal monophosphate prodrugs,
each R⁴ are independently selected from aryl, heteroaryl, arylalkyl, alkylaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl, and C₂₋₁₀ alkynyl, each of which is unsubstituted or independently substituted with one or more substituents selected from the group consisting of R⁵, OH, OR⁵, SR⁵, S(O)R⁵, SO₂R⁵, C(O)R⁵, C(O)OR⁵, OC(O)R⁵, OC(O)OR⁵, NH₂, NHR⁵, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, COOH, C(O)H, C(N)NH₂, C(N)NHR⁵, C(N)N(R⁵)₂, C(N)OH, C(N)OCH₃, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
each R⁵ are independently aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁶, OH, OR⁶, SR⁶, S(O)R⁶, SO₂R⁶, C(O)R⁶, C(O)OR⁶, OC(O)R⁶, OC(O)OR⁶, NH₂, NHR⁶, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, COOH, C(O)H, C(N)NH₂, C(N)NHR⁶, C(N)N(R⁶)₂, C(N)OH, C(N)OCH₃, CN, N₃, NO₂, CF₃, CF₂CF₃, OCF₃, OCF₂CF₃, F, Cl, Br, I, P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
each R⁶ are independently aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of C₁₋₁₀ alkyl, C₂₋₁₀ alkenyl, C₂₋₁₀ alkynyl, OH, NH₂, C(O)NH₂, C(O)NHOH, SO₂NH₂, COOH, C(O)H, C(N)NH₂, C(N)OH, C(N)OCH₃, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs.

Pharmaceutically-acceptable salts and prodrugs of these compounds are also intended to be within the scope of the invention.

Representative R² moieties are shown below:

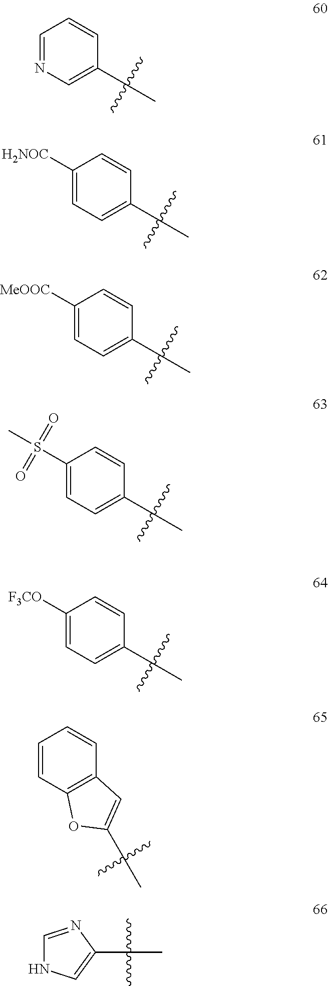

Representative R³ moieties are shown below:

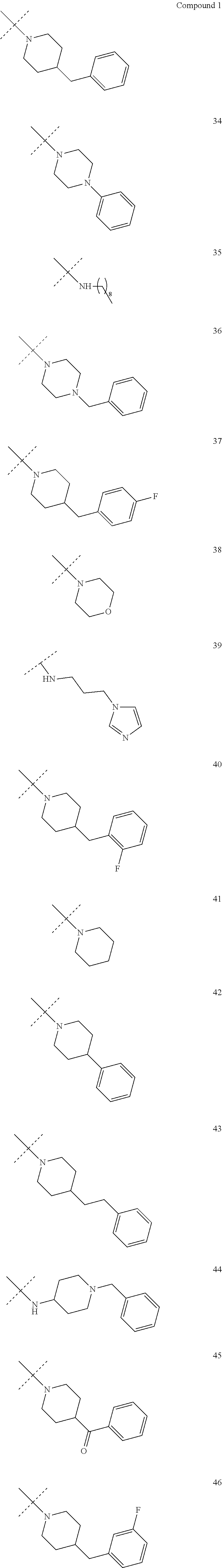

In one embodiment, one of X and Z is —C(O)—, —SO₂—, or —NC(O)—, and the other is —NH—, —N(NH₂)—, —NH(OH)—, —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, or —O—.

In another embodiment, one of X and Z is —C(O)—, —SO₂—, or —N(C(O)—, and the other is —CH₂—, —CH(C₁₋₆ alkyl)-, C(alkyl)₂-, —CH(C₃₋₈ cycloalkyl)-, —CH(C₂₋₆ alkenyl, —CH(C₂₋₆ alkynyl)-, —CH(aryl)-, —CH(heteroaryl)-, —CF₂—, —CCl₂—, —CH(CF₃)—, —CH(OH)—, —CH(OAlkyl)-, —CH(NH₂)—, —CH(NHAlkyl)-, or —CH(C(O)NH₂)—.

In another embodiment, one of X and Z is —NH—, —N(NH₂)—, —NH(OH)—, —N(alkyl)-, or —O— and the other is —CH₂—, —CH(C₁₋₆ alkyl)-, C(alkyl)₂-, —CH(C₃₋₈ cycloalkyl)-, —CH(C₂₋₆ alkenyl, —CH(C₂₋₆ alkynyl)-, —CH(aryl)-, —CH(heteroaryl)-, —CF₂—, —CCl₂—, —CH(CF₃)—, —CH(OH)—, —CH(OAlkyl)-, —CH(NH₂)—, —CH(NHAlkyl)-, or —CH(C(O)NH₂)—.

In a third embodiment, one of X and Z is —NH—, —N(NH₂)—, —NH(OH)—, —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, and the other is —C(O)— or —SO₂—.

In a fourth embodiment, Y is —NH, —N(NH₂)—, —NH(OH)—, —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, or —O—.

In a fifth embodiment, Y is —NH, —N(NH₂)—, —NH(OH)—, —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, In a sixth embodiment, one of R¹ and R² is H, —CH₂-phosphonate, —CH₂O-phosphate, wherein the term phosphate includes monophosphate, diphosphate, triphosphate, and stabilized phosphate prodrugs, and the term phosphonate includes the same prodrugs that are present in the phosphate prodrugs.

In a seventh embodiment, one of R¹ and R² is H, —CH₂P(O)(OH)₂, —CH₂P(O)(OH)(OR⁶), —CH₂P(O)(OR⁶)₂, —CH₂P(O)(OR⁶)(NR⁶), —CH₂P(O)(NR⁶)₂, —CH₂P(O)(OH)(OC₁₋₁₀ alkyl-O—C₁₋₂₀ alkyl), or a —CH₂-cycloSal monophosphate prodrug.

In one aspect of this embodiment, one of R¹ and R² is a phosphonate, a phosphoramidate, a cycloSal monophosphate prodrug, or has the formula —CH₂P(O)(OH)(OC₁₋₁₀ alkyl-O—C₁₋₂₀ alkyl).

In a preferred embodiment, one of R¹ and R² is —C(O)NHR⁴, —C(O)N(R⁴)₂,

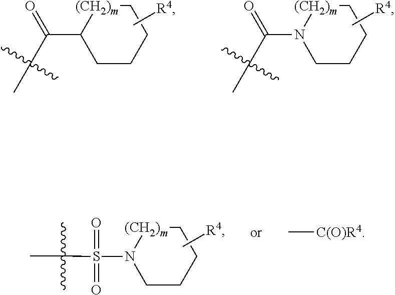

wherein R⁴ is C₁₋₁₀ alkyl, C₃₋₁₀ cycloalkyl, C₂₋₁₀ alkenyl, C₂₋₁₀ alkynyl, arylalkyl, alkylaryl, C₁₋₁₀ halo alkyl, C₁₋₁₀ alkyl-aryl, or C₁₋₁₀ haloalkyl-aryl and m is 0, 1 or 2. In specific embodiments, R⁴ is C₁₋₁₀ alkyl-aryl, and benzyl is a particularly preferred R⁴ substituent.

In another embodiment, one of R¹ and R² is —C(O)—C₁₋₁₀ alkyl, —C(O)-alkylaryl, —C(O)— heterocyclyl-alkylaryl, —C(O)-heterocyclyl-CH₂-aryl, —C(O)-heterocyclyl-CF₂-aryl, —C(O)— cycloalkyl-alkylaryl, —C(O)NHC₁₋₁₀ alkyl, —C(O)NH-alkylaryl, —C(O)NH-heterocyclyl-alkylaryl, —C(O)NH-heterocyclyl-CF₂-aryl, —C(O)NH-cycloalkyl-alkylaryl, —SO₂—C₁₋₁₀ alkyl, —SO₂-alkylaryl, —SO₂-heterocyclyl-alkylaryl, —SO₂-heterocyclyl-CF₂-aryl, or —SO₂-cycloalkyl-alkylaryl.

The specific variables shown above can also be used in connection with any of Formulas B through H, discussed in more detail below.

Representative compounds include the following:

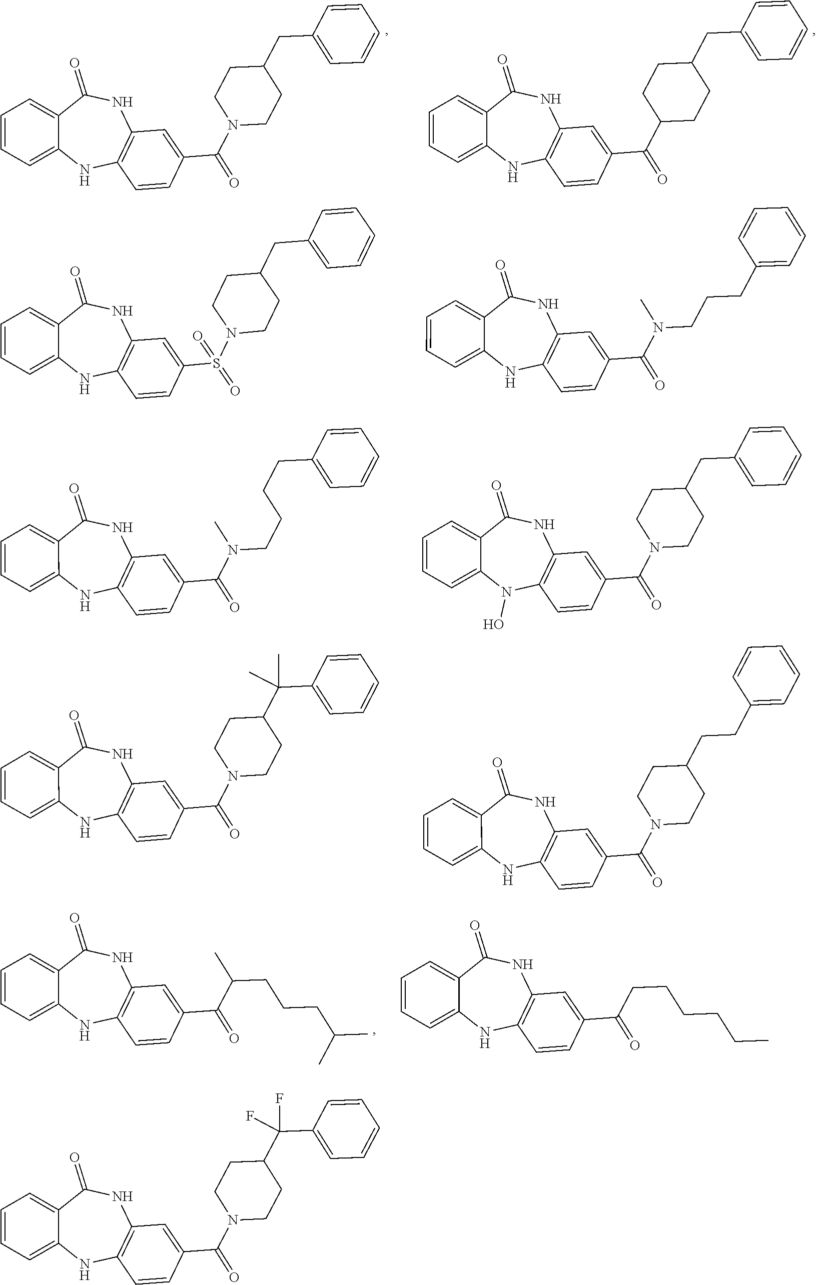

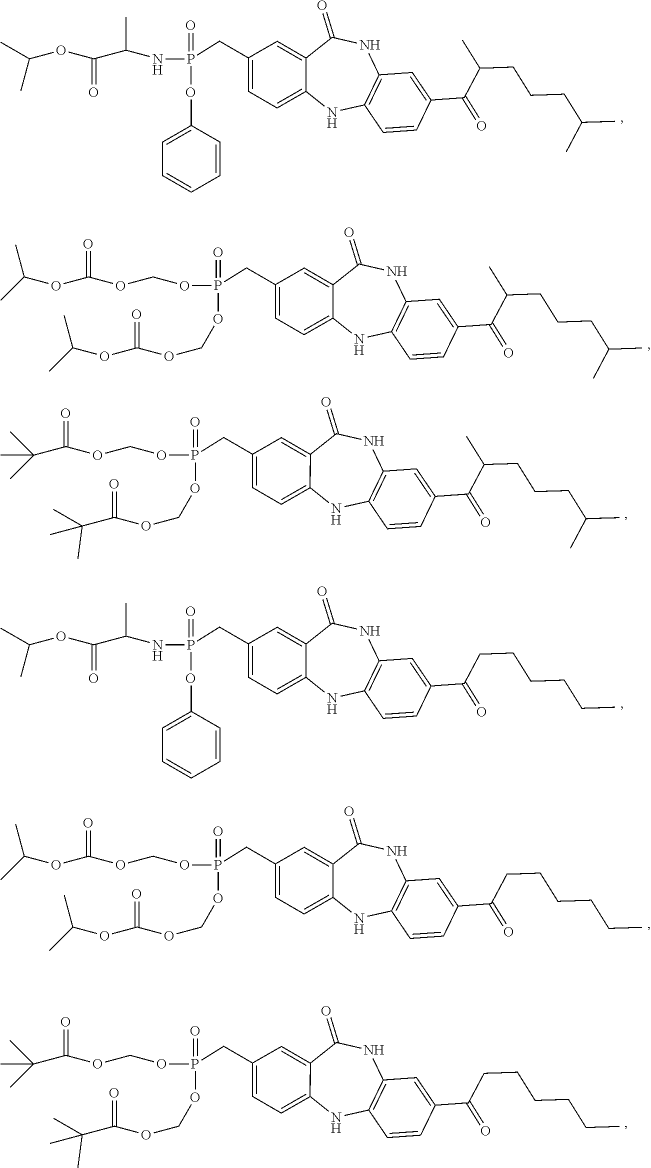

or a pharmaceutically-acceptable salt or prodrug thereof.

A particularly referred compound has the formula:

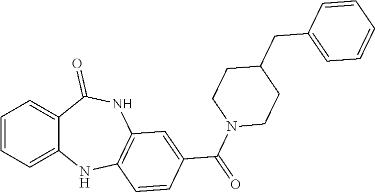 [SMILES: O=C1Nc2cc(C(=O)N3CCC(Cc4ccccc4)CC3)ccc2Nc2ccccc21]

or a pharmaceutically acceptable salt or prodrug thereof.

In other embodiments, the compounds have one of the following formulas:

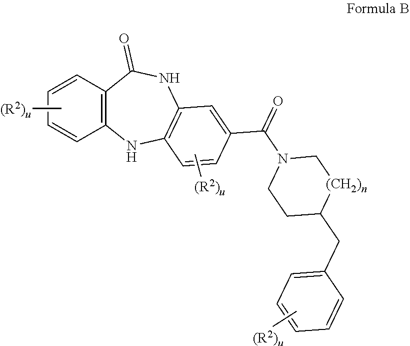

and pharmaceutically-acceptable salts and prodrugs thereof, wherein R² and u are as defined above with respect to Formula A, except that u may be 0.

One representative compound has the following formula:

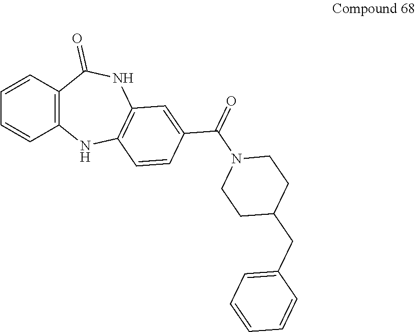 [SMILES: O=C1Nc2cc(C(=O)N3CCC(Cc4ccccc4)CC3)ccc2Nc2ccccc21]

and pharmaceutically-acceptable salts and prodrugs thereof, where R² and u are defined as described above for Formula A, except that u may be 0.

In another embodiment, the compounds have one of the following formulas:

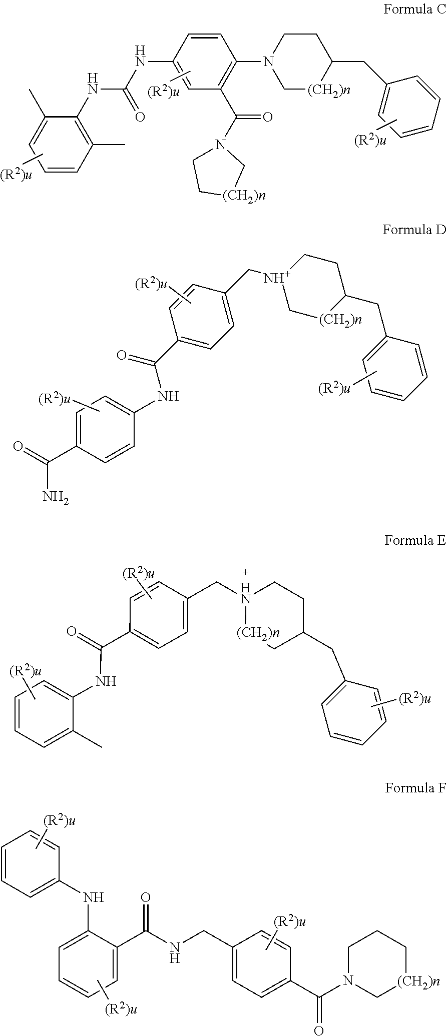

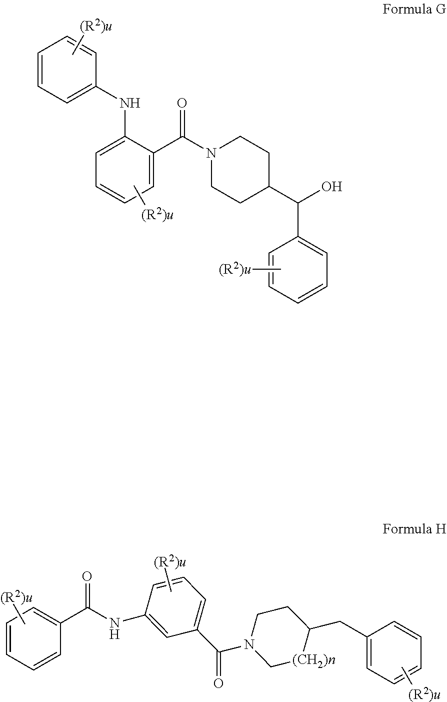

and pharmaceutically-acceptable salts and prodrugs thereof, wherein R² and u are as defined above with respect to Formula A, except that u may be 0, and n is 0, 1, or 2.

### III. Stereoisomerism and Polymorphism

The compounds described herein can have asymmetric centers and occur as racemates, racemic mixtures, individual diastereomers or enantiomers, with all isomeric forms being included in the present invention. Compounds of the present invention having a chiral center can exist in and be isolated in optically active and racemic forms. Some compounds can exhibit polymorphism. The present invention encompasses racemic, optically-active, polymorphic, or stereoisomeric forms, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme;
iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### IV. Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts can also be formed, including but not limited to, sulfate, nitrate, bicarbonate and carbonate salts. For certain transdermal applications, it can be preferred to use fatty acid salts of the compounds described herein. The fatty acid salts can help penetrate the stratum corneum. Examples of suitable salts include salts of the compounds with stearic acid, oleic acid, lineoleic acid, palmitic acid, caprylic acid, and capric acid.

Pharmaceutically acceptable salts can be obtained using standard procedures well known in the art, for example by reacting a sufficiently basic compound such as an amine with a suitable acid, affording a physiologically acceptable anion. In those cases where a compound includes multiple amine groups, the salts can be formed with any number of the amine groups. Alkali metal (e.g., sodium, potassium or lithium) or alkaline earth metal (e.g., calcium) salts of carboxylic acids can also be made.

A prodrug is a pharmacological substance that is administered in an inactive (or significantly less active) form and subsequently metabolized in vivo to an active metabolite. Getting more drug to the desired target at a lower dose is often the rationale behind the use of a prodrug and is generally attributed to better absorption, distribution, metabolism, and/or excretion (ADME) properties. Prodrugs are usually designed to improve oral bioavailability, with poor absorption from the gastrointestinal tract usually being the limiting factor. Additionally, the use of a prodrug strategy can increase the selectivity of the drug for its intended target thus reducing the potential for off target effects.

### V. Methods of Treatment

Hosts can be treated by administering to the patient an effective amount of the active compound or a pharmaceutically acceptable prodrug or salt thereof, optionally in the presence of a pharmaceutically acceptable carrier or diluent. The active materials can be administered by any appropriate route, for example, orally, parenterally, intravenously, intradermally, transdermally, subcutaneously, or topically, in liquid or solid form. Details of administration are provided in pharmaceutical compositions.

The compounds can be used for treating, preventing, reducing the susceptibility to, reducing the severity of, or delaying the progression of pancreatitis, sarcopenia, stroke, and traumatic brain injury, which are associated with FGF21, as well as glioblastomas and other disorders associated with miR-122. In some embodiments, the compounds are administered with other pharmaceutical agents useful for treating these disorders.

### Treatment of Glioblastoma

Glioblastoma (GBM), a mostly lethal brain tumor, acquires large amounts of free fatty acids (FAs) to promote cell growth. While high levels of FAs typically result in lipotoxicity, research has suggested that GBM avoids lipotoxocity by upregulating diacylglycerol-acyltransferase 1 (DGAT1) to store excess FAs as triglycerides and lipid droplets (Cheng et al., 2020, Cell Metabolism 32, 229-242 (Aug. 4, 2020)).

Cheng looked at inhibiting DGAT1 as a way to disrupt lipid homeostasis, noting that DGAT1 inhibition resulted in excessive FAs moving into mitochondria for oxidation. This led to the generation of high levels of reactive oxygen species (ROS), mitochondrial damage, cytochrome c release, and tumor cell apoptosis. In an animal model of GBM, Cheng showed that targeting DGAT1 blocked lipid droplet formation, induced tumor cell apoptosis, and markedly suppressed GBM growth, and suggested that targeting DGAT1 could be a promising therapeutic approach for GBM. However, DGAT1 inhibitors are accompanied by gastrointestinal adverse events such as nausea, diarrhea, and vomiting (DeVita and Pinto, “Current status of the research and development of diacylglycerol O-acyltransferase 1 (DGAT1) inhibitors,” J Med Chem. 56(24):9820-5 (2013), and the DGAT1 inhibitor Cheng evaluated (DGAT1 inhibitor A-922500) was unable to cross the blood-brain barrier, so would be unable to treat GBM. It would therefore be advantageous to provide alternatives to DGAT1 inhibitors as a way to inhibit lipid droplet formation, and thus treat GBM.

There is an association between miR-122 and lipid droplet formation (Wu et al., “MicroRNA-122 Inhibits Lipid Droplet Formation and Hepatic Triglyceride Accumulation via Yin Yang 1,” Cell Physiol Biochem., 44(4):1651-1664 (2017)). Wu disclosed that an increase in intracellular lipid droplet formation and hepatic triglyceride (TG) content can result in nonalcoholic fatty liver disease, miR-122 was downregulated in free fatty acid (FFA)-induced steatotic hepatocytes, and streptozotocin and high-fat diet (STZ-HFD) induced nonalcoholic steatohepatitis (NASH) in mice. Transfection of hepatocytes with miR-122 mimics before FFA induction inhibited lipid droplet formation and TG accumulation in vitro.

The ROR-α agonist compounds described herein increase circulating miR-122 levels. Since miR-122 can cross the blood-brain barrier, administration of these compounds increases miR-122 levels in the brain. As such, the compounds can block lipid droplet formation, induce tumor cell apoptosis, and suppress GBM growth.

### VI. Combination or Alternation Therapy

In one embodiment, a compound of Formula (A) or a pharmaceutically acceptable derivative thereof, can be employed alone, in combination with one or more compounds of formula (A) or a pharmaceutically acceptable derivative thereof, or in combination with at least one other agent in use for treating conditions associated with ROR.

In certain embodiments, a compound of Formula (A) for treatment of pancreatitis in combination with agents such as, but not limited to, analgesics, such as Acetaminophen Ibuprofen, Hydrocodone, Tramadol, or Naproxen, enzyme pills to help with digestion, vitamins, such as vitamins A, B12, D, E, and/or K if the patient suffers from malabsorption, and/or STAT3 (Signal Transducer and Activator of Transcription) inhibitors, such as Niclosamide, WP1066 (WPD Pharma), OPB-51602 (Medkoo Biosciences) and S3I-201 (Santa Cruz Biotechnology), as well as inhibitors of other members of the STAT protein family, including STAT1, STAT2, STAT4, STAT5 (STAT5A and STAT5B), and STAT6.

The course of therapy can be followed, for example, with blood tests to look for elevated levels of pancreatic enzymes, stool tests in chronic pancreatitis to measure levels of fat that could suggest the patient's digestive system is not adequately absorbing nutrients, computerized tomography (CT) scan to look for gallstones and assess the extent of pancreas inflammation, abdominal ultrasound to look for gallstones and pancreas inflammation, endoscopic ultrasound to look for inflammation and blockages in the pancreatic duct or bile duct, and/or magnetic resonance imaging (MRI) to look for abnormalities in the gallbladder, pancreas and ducts.

When used to treat sarcopenia, the compounds can be co-administered with Urocortin II, hormones, such as testosterone or growth hormone, STAT3 inhibitors, such as Niclosamide, WP1066 (WPD Pharma), OPB-51602 (Medkoo Biosciences) and S3I-201 (Santa Cruz Biotechnology), as well as inhibitors of other members of the STAT protein family, including STAT1, STAT2, STAT4, STAT5 (STAT5A and STAT5B), and STAT6, and medications for treating metabolic syndrome (including, insulin-resistance, obesity, and hypertension), such as metformin and other AMPK agonists.

When used to treat glioblastoma, the compounds can be administered with other treatments for glioblastoma. One or more of the other active agents described below, in any combination, can be administered to aggressively treat GBM.

Treating glioblastoma has historically been very difficult, due to several complicating factors. The tumor cells are very resistant to conventional therapies, the brain is susceptible to damage from conventional therapy, and has a very limited capacity to repair itself, and many drugs cannot cross the blood-brain barrier to act on the tumor.

Temozolomide (TMZ) is one example of a drug which can be used to treat glioblastoma multiforme. It can be administered orally or intravenously.

Cannabinoids (whether in the form of tetrahydrocannabinol (THC), the synthetic analogue nabilone, CBD, CBG, or other cannabinoids), can be co-administered. These compounds can combat nausea and vomiting induced by chemotherapy, stimulate appetite, lessen anguish and pain, and inhibit growth and angiogenesis in malignant gliomas. Cannabinoids can attack the neoplastic stem cells of glioblastomas, inducing their differentiation into more mature (and therefore more “treatable”) cells.

Berberine, an isoquinoline alkaloid, is one example of a compound which can be co-administered. The antitumor effect of berberine on glioblastoma cells is believed to involve induction of cellular senescence, inhibition of the RAF-MEK-ERK signaling pathway and/or downregulation of EGFR.

Direct nose-to-brain drug delivery can be used to achieve higher, and hopefully more effective, drug concentrations in the brain, of the compounds described herein, and also with the additional active agents described herein. The natural compound perillyl alcohol can be administered via intranasal delivery, for example, as an aerosol.

GBM tumors contain zones of tissue exhibiting hypoxia, which are highly resistant to radiotherapy. Radiosensitizers can be co-administered, along with radiotherapy. Oxygen diffusion-enhancing compounds such as trans-sodium crocetinate are examples of radiosensitizers. Boron neutron capture therapy has been tested as an alternative treatment for glioblastoma.

Anticonvulsants such as phenytoin can be co-administered, typically after a seizure occurs. Corticosteroids, usually dexamethasone, can reduce peritumoral edema (through rearrangement of the blood-brain barrier), diminishing mass effect and lowering intracranial pressure, with a decrease in headache or drowsiness.

The compounds described herein can also be combined with chimeric antigen receptor (CAR) T cell therapy. CAR T cells using CLTX as the targeting domain (CLTX-CAR T cells) mediate potent anti-GBM activity and efficiently target tumors lacking expression of other GBM-associated antigens (Wang et al., “Chlorotoxin-directed CAR T cells for specific and effective targeting of glioblastoma,” Science Translational Medicine, Vol. 12, Issue 533, eaaw2672 (March 2020). CAR T-cell therapy using IL13Rα2, Her2/CMV, EGFRvIII, CSPG4, NKG2DL, CD19, and CD133 as the targeting domain can also be used. Representative therapies include Novartis' Kymriah and Gilead Sciences' Yescarta.

MP-Pt(IV) is a MAOB-sensitive mitochondrial-specific prodrug for treating glioblastoma, which can also be combined with the compounds described herein.

RIPGBM (N-[1,4-Dihydro-1,4-dioxo-3-[(phenylmethyl)amino]-2-naphthalenyl]-N-[(4-fluorophenyl)methyl]acetamide) is an RIPK2 modulator, and is a prodrug of cRIPGM. The compound selectively induces apoptosis in glioblastoma multiforme cancer stem cell lines, and is orally bioavailable and brain penetrant. The structure is shown below:

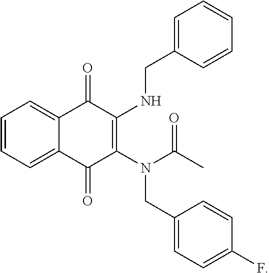 [SMILES: CC(=O)N(Cc1ccc(F)cc1)C1=C(NCc2ccccc2)C(=O)c2ccccc2C1=O]

Kisquali® (Ribociclib), an inhibitor of cyclin D1/CDK4 and CDK6, is another anti-cancer drug that can be combined with the compounds described herein.

Given the aggressive nature of GBM, it is contemplated that one or more of these active agents can be combined with the compounds described herein, to attack GBM via multiple biological pathways.

When used to treat traumatic brain injury, the compounds can be co-administered with Tranexamic acid (when administered shortly after the injury), sedatives, analgesics and paralytic agents while managing intracranial pressure (ICP), anti-seizure medications, such as phenytoin and leviteracetam, and norepinephrine or similar drugs to help maintain cerebral perfusion, intranasal insulin, as described in U.S. Pat. No. 10,314,911, and VLA-1 (Very Late Activation Antigen-I) antagonists.

When used to treat stroke, the compounds can be co-administered with compounds that inhibit blood clot formation, such as blood thinners, or compounds that break up existing blood clots, such as tissue plasminogen activator (TPA), Integrilin (eptifibatide), abciximab (ReoPro) or tirofiban (Aggrastat).

Blood thinners prevent blood clots from forming, and keep existing blood clots from getting larger. There are two main types of blood thinners. Anticoagulants, such as heparin or warfarin (also called Coumadin), slow down biological processes for producing clots, and antiplatelet aggregation drugs, such as Plavix, aspirin, prevent blood cells called platelets from clumping together to form a clot.

By way of example, Integrilin® is typically administered at a dosage of 180 mcg/kg intravenous bolus administered as soon as possible following diagnosis, with 2 mcg/kg/min continuous infusion (following the initial bolus) for up to 96 hours of therapy.

Representative platelet aggregation inhibitors include glycoprotein IIB/IIIA inhibitors, phosphodiesterase inhibitors, adenosine reuptake inhibitors, and adenosine diphosphate (ADP) receptor inhibitors. These can optionally be administered in combination with an anticoagulant.

Representative anti-coagulants include coumarins (vitamin K antagonists), heparin and derivatives thereof, including unfractionated heparin (UFH), low molecular weight heparin (LMWH), and ultra-low-molecular weight heparin (ULMWH), synthetic pentasaccharide inhibitors of Factor Xa, including Fondaparinux, Idraparinux, and Idrabiotaparinux, directly acting oral anticoagulants (DAOCs), such as dabigatran, rivaroxaban, apixaban, edoxaban and betrixaban, and antithrombin protein therapeutics/thrombin inhibitors, such as bivalent drugs hirudin, lepirudin, and bivalirudin and monovalent argatroban.

Representative platelet aggregation inhibitors include pravastatin, Plavix (clopidogrel bisulfate), Pletal (cilostazol), Effient (prasugrel), Aggrenox (aspirin and dipyridamole), Brilinta (ticagrelor), caplacizumab, Kengreal (cangrelor), Persantine (dipyridamole), Ticlid (ticlopidine), Yosprala (aspirin and omeprazole).

The compounds can also be co-administered with neuroprotective agents, such as thrombolytic agents, erythropoiesis-stimulating agents, such as erythropoietin, darbepoetin, and epoetin alfa, ET_{B} receptor agonists, such as IRL-1620, ET_{A} receptor agonists, such as sulfosoxazole, clazosentan, atrasentan, tezosentan, bosentan, sitaxsentan, enrasentan, BMS 207940, BMS 193884, BMS 182874, J 104132, VML 588/Ro 61 1790, T-0115, TAK 044, BQ 788, TBC2576, TBC3214, PD180988, ABT 546, SB247083, RPR118031A, and BQ123, as well as argatroban, alfimeprase, tenecteplase, ancrod, sildenafil, insulin, insulin growth factor, magnesium sulfate, human serum albumin, caffeinol, microplasmin, a statin, eptifibatide, tinzaparin, enecadin, citicoline, edaravone, cilostazol, and mixtures thereof.

Other agents for use in combination for conditions associated with ROR are, but not limited to, the following: cholesterol biosynthesis inhibitors (HMG CoA reductase inhibitors, e.g., lovastatin, simvastatin, pravastatin, fluvastatin, atorvastatin, cerivastatin, nisvastatin and rivastatin); squalene epoxidase inhibitors (e.g. terbinafine); plasma HDL-raising agents (e.g. CETP inhibitors e.g. anacetrapib, R1658); human peroxisome proliferator activated receptor (PPAR) gamma agonists (e.g., thiazolidinediones e.g. rosiglitazone, troglitazone, and pioglitazone); PPAR alpha agonists (e.g. clofibrate, fenofibrate, and gemfibronzil); PPAR dual alpha/gamma agonists (e.g. muraglitazar, aleglitazar, peliglitazar, elafibranor); farnesoid X receptor (FXR) modulators (e.g., obeticholic acid, LMB763, LJN45, etc.); bile acid sequestrants (e.g., anion exchange resins, or quaternary amines (e.g. cholestyramine or colestipol)); bile acid transport inhibitors (BATi); nicotinic acid, niacinamide; cholesterol absorption inhibitors (e.g. ezetimibe); acyl-coenzyme A:cholesterol O-acyl transferase (ACAT) inhibitors (e.g., avasimibe); selective estrogen receptor modulators (e.g. raloxifene or tamoxifen); LXR alpha or beta agonists, antagonists or partial agonists (e.g., 22(R)-hydroxycholesterol, 24(S)-hydroxycholesterol, T0901317 or GW3965); microsomal triglyceride transfer protein (MTP) inhibitors, anti-diabetes agents such as, e.g. insulin and insulin analogs (e.g. LysPro insulin, inhaled formulations comprising insulin; sulfonylureas and analogues (e.g. tolazamide, chlorpropamide, glipizide, glimepiride, glyburide, glibenclamide, tolbutamide, acetohexamide, glypizide), biguanides (e.g., metformin or metformin hydrochloride, phenformin, buformin) alpha2-antagonists and imidazolines (e.g. midaglizole, isaglidole, deriglidole, idazoxan, efaroxan, fluparoxan), thiazolidinediones (e.g., pioglitazone hydrochloride, rosiglitazone maleate, ciglitazone, troglitazone or balaglitazone), alpha-glucosidase inhibitors (e.g. miglitol, acarbose, epalrestat, or voglibose), meglitinides (e.g. repaglinide or nateglinide), DPP-4 inhibitors (e.g., sitagliptin phosphate, saxagliptin, vildagliptin, alogliptin or denagliptin), incretins (e.g. glucagon-like peptide-1 (GLP-1) receptor agonists (e.g. Exenatide (Byetta™), NN2211 (Liraglutide), GLP-1(7-36) amide and its analogs, GLP-1(7-37) and its analogs, AVE-0010 (ZP-10), R1583 (Taspoglutide), GSK-716155 (albiglutide, GSK/Human Genome Sciences), BRX-0585 (Pfizer/Biorexis) and CJC-1134-PC (Exendin-4:PC-DAC™ and glucose-dependent insulinotropic peptide (GIP)); amylin agonists (e.g. pramlintide, AC-137); insulin secretagogues (e.g. linogliride, nateglinide, repaglinide, mitiglinide calcium hydrate or meglitinide); SGLT-2 inhibitors (e.g. dapagliflozin (BMS), sergliflozin (Kissei), AVE 2268 (Sanofi-Aventis); Glucokinase activators such as the compounds disclosed in e.g. WO 00/58293 A1; anti-obesity agents such as nerve growth factor agonist (e.g. axokine), growth hormone agonists (e.g. AOD-9604), adrenergic uptake inhibitors (e.g. GW-320659), 5-HT (serotonin) reuptake/transporter inhibitors (e.g. Prozac), 5-HT/NA (serotonin/noradrenaline) reuptake inhibitors (e.g. sibutramine), DA (dopamine) reuptake inhibitors (e.g. Buproprion), 5-HT, NA and DA reuptake blockers, steroidal plant extracts (e.g. P57), NPY1 or 5 (neuropeptide Y Y1 or Y5) antagonists, NPY2 (neuropeptide Y Y2) agonists, MC4 (melanocortin 4) agonists, CCK-A (cholecystokinin-A) agonists, GHSR1a (growth hormone secretagogue receptor) antagonist/inverse agonists, ghrelin antibody, MCH1R (melanin concentrating hormone 1R) antagonists (e.g. SNAP 7941), MCH2R (melanin concentrating hormone 2R) agonist/antagonists, H3 (histamine receptor 3) inverse agonists or antagonists, H1 (histamine 1 receptor) agonists, FAS (Fatty acid synthase) inhibitors, ACC-1 (acetyl-CoA carboxylase-1) inhibitors, 33 (beta adrenergic receptor 3) agonists, DGAT-2 (diacylglycerol acyltransferase 2) inhibitors, DGAT-1 (diacylglycerol acyltransferase 1) inhibitors, CRF (corticotropin releasing factor) agonists, Galanin antagonists, UCP-1 (uncoupling protein-1), 2 or 3 activators, leptin or a leptin derivatives, opioid antagonists, orexin antagonists, BRS3 agonists, GLP-1 (glucagons-like peptide-1) agonists, IL-6 agonists, a-MSH agonists, AgRP antagonists, BRS3 (bombesin receptor subtype 3) agonists, 5-HT1B agonists, POMC antagonists, CNTF (ciliary neurotrophic factor or CNTF derivative), NN2211, Topiramate, glucocorticoid antagonist, Exendin-4 agonists, 5-HT2C (serotonin receptor 2C) agonists (e.g. Lorcaserin), PDE (phosphodiesterase) inhibitors, fatty acid transporter inhibitors, dicarboxylate transporter inhibitors, glucose transporter inhibitors, CB-1 (cannabinoid-1 receptor) inverse agonists or antagonists (e.g. SR141716), lipase inhibitors (e.g., orlistat); cyclooxygenase-2 (COX-2) inhibitors (e.g. rofecoxib and celecoxib); thrombin inhibitors (e.g., heparin, argatroban, melagatran, dabigatran); platelet aggregation inhibitors (e.g. glycoprotein IIb/IIIa fibrinogen receptor antagonists or aspirin); vitamin B6 and pharmaceutically acceptable salts thereof; vitamin B 12; vitamin E; folic acid or a pharmaceutically acceptable salt or ester thereof; antioxidant vitamins such as C and E and beta carotene; beta blockers (e.g. angiotensin II receptor antagonists such as losartan, irbesartan or valsartan; antiotensin converting enzyme inhibitors such as enalapril and captopril; calcium channel blockers such as nifedipine and diltiazam; endothelian antagonists; aspirin; fatty-acid/bile-acid conjugates (Aramchol); caspase inhibitors (emricasan); immunomodulators (Cenicriviroc, etc.); thyroid hormone receptor modulators (MB07811, MGL-3196, etc.); agents other than LXR ligands that enhance ATP-Binding Cassette Transporter-Al gene expression; and bisphosphonate compounds (e.g., alendronate sodium).

In certain embodiments, a compound of Formula (A) in combination with at least one other agent that modifies host metabolism such as, but not limited to, clarithromycin, cobicistat, indinavir, itraconazole, ketoconazole, nefazodone, ritonavir, saquinavir, suboxone, telithromycin, aprepitant, erythromycin, fluconazole, verapamil, diltiazem, cimetidine, amiodarone, boceprevir, chloramphenicol, ciprofloxacin, delaviridine, diethyl-dithiocarbamate, fluvoxamine, gestodene, imatinib, mibefradil, mifepristone, norfloxacin, norfluoxetine, telaprevir, and voriconazole.

### VII. Pharmaceutical Compositions

Hosts, including but not limited to humans, affected by pancreatitis, stroke, traumatic brain injury or sarcopenia, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound for will be in the range of between about 0.01 and about 10 mg/kg, more generally, between about 0.1 and 5 mg/kg, and, preferably, between about 0.5 and about 2 mg/kg, of body weight of the recipient per day. The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to but not limited to one containing 7 to 600 mg, preferably 70 to 600 mg of active ingredient per unit dosage form. An oral dosage of 5-400 mg is usually convenient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antiviral compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

### Transdermal Formulations

In some embodiments, the compositions are present in the form of transdermal formulations, such as that used in the FDA-approved agonist rotigitine transdermal (Neupro patch). Another suitable formulation is that described in U.S. Publication No. 20080050424, entitled “Transdermal Therapeutic System for Treating Parkinsonism.” This formulation includes a silicone or acrylate-based adhesive, and can include an additive having increased solubility for the active substance, in an amount effective to increase dissolving capacity of the matrix for the active substance.

The transdermal formulations can be single-phase matrices that include a backing layer, an active substance-containing self-adhesive matrix, and a protective film to be removed prior to use. More complicated embodiments contain multiple-layer matrices that may also contain non-adhesive layers and control membranes. If a polyacrylate adhesive is used, it can be crosslinked with multivalent metal ions such as zinc, calcium, aluminum, or titanium ions, such as aluminum acetylacetonate and titanium acetylacetonate.

When silicone adhesives are used, they are typically polydimethylsiloxanes. However, other organic residues such as, for example, ethyl groups or phenyl groups may in principle be present instead of the methyl groups. Because the active compounds are amines, it may be advantageous to use amine-resistant adhesives. Representative amine-resistant adhesives are described, for example, in EP 0 180 377.

Representative acrylate-based polymer adhesives include acrylic acid, acrylamide, hexylacrylate, 2-ethylhexylacrylate, hydroxyethylacrylate, octylacrylate, butylacrylate, methylacrylate, glycidylacrylate, methacrylic acid, methacrylamide, hexylmethacrylate, 2-ethylhexylmethacrylate, octylmethacrylate, methylmethacrylate, glycidylmethacrylate, vinylacetate, vinylpyrrolidone, and combinations thereof.

The adhesive must have a suitable dissolving capacity for the active substance, and the active substance most be able to move within the matrix, and be able to cross through the contact surface to the skin. Those of skill in the art can readily formulate a transdermal formulation with appropriate transdermal transport of the active substance.

Certain pharmaceutically acceptable salts tend to be more preferred for use in transdermal formulations, because they can help the active substance pass the barrier of the stratum corneum. Examples include fatty acid salts, such as stearic acid and oleic acid salts. Oleate and stearate salts are relatively lipophilic, and can even act as a permeation enhancer in the skin.

Permeation enhancers can also be used. Representative permeation enhancers include fatty alcohols, fatty acids, fatty acid esters, fatty acid amides, glycerol or its fatty acid esters, N-methylpyrrolidone, terpenes such as limonene, alpha-pinene, alpha-terpineol, carvone, carveol, limonene oxide, pinene oxide, and 1,8-eucalyptol.

The patches can generally be prepared by dissolving or suspending the active agent in ethanol or in another suitable organic solvent, then adding the adhesive solution with stirring. Additional auxiliary substances can be added either to the adhesive solution, the active substance solution or to the active substance-containing adhesive solution. The solution can then be coated onto a suitable sheet, the solvents removed, a backing layer laminated onto the matrix layer, and patches punched out of the total laminate.

### Nanoparticulate Compositions

The compounds described herein can also be administered in the form of nanoparticulate compositions. In one embodiment, controlled release nanoparticulate formulations comprise a nanoparticulate active agent to be administered and a rate-controlling polymer which prolongs the release of the agent following administration. In this embodiment, the compositions can release the active agent, following administration, for a time period ranging from about 2 to about 24 hours or up to 30 days or longer. Representative controlled release formulations including a nanoparticulate form of the active agent are described, for example, in U.S. Pat. No. 8,293,277.

Nanoparticulate compositions can comprise particles of the active agents described herein, having a non-crosslinked surface stabilizer adsorbed onto, or associated with, their surface.

The average particle size of the nanoparticulates is typically less than about 800 nm, more typically less than about 600 nm, still more typically less than about 400 nm, less than about 300 nm, less than about 250 nm, less than about 100 nm, or less than about 50 nm. In one aspect of this embodiment, at least 50% of the particles of active agent have an average particle size of less than about 800, 600, 400, 300, 250, 100, or 50 nm, respectively, when measured by light scattering techniques.

A variety of surface stabilizers are typically used with nanoparticulate compositions to prevent the particles from clumping or aggregating. Representative surface stabilizers are selected from the group consisting of gelatin, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, poloxamines, poloxamine 908, dialkylesters of sodium sulfosuccinic acid, sodium lauryl sulfate, an alkyl aryl polyether sulfonate, a mixture of sucrose stearate and sucrose distearate, p-isononylphenoxypoly-(glycidol), SA9OHCO, decanoyl-N-methylglucamide, n-decyl-D-glucopyranoside, n-decyl-D-maltopyranoside, n-dodecyl-D-glucopyranoside, n-dodecyl-D-maltoside, heptanoyl-N-methylglucamide, n-heptyl-D-glucopyranoside, n-heptyl-D-thioglucoside, n-hexyl-D-glucopyranoside, nonanoyl-N-methylglucamide, n-nonyl-D-glucopyranoside, octanoyl-N-methylglucamide, n-octyl-D-glucopyranoside, and octyl-D-thioglucopyranoside. Lysozymes can also be used as surface stabilizers for nanoparticulate compositions. Certain nanoparticles such as poly(lactic-co-glycolic acid) (PLGA)-nanoparticles are known to target the liver when given by intravenous (IV) or subcutaneously (SQ).

Representative rate controlling polymers into which the nanoparticles can be formulated include chitosan, polyethylene oxide (PEO), polyvinyl acetate phthalate, gum arabic, agar, guar gum, cereal gums, dextran, casein, gelatin, pectin, carrageenan, waxes, shellac, hydrogenated vegetable oils, polyvinylpyrrolidone, hydroxypropyl cellulose (HPC), hydroxyethyl cellulose (HEC), hydroxypropyl methylcellulose (HPMC), sodium carboxymethylcellulose (CMC), poly(ethylene) oxide, alkyl cellulose, ethyl cellulose, methyl cellulose, carboxymethyl cellulose, hydrophilic cellulose derivatives, polyethylene glycol, polyvinylpyrrolidone, cellulose acetate, cellulose acetate butyrate, cellulose acetate phthalate, cellulose acetate trimellitate, polyvinyl acetate phthalate, hydroxypropylmethyl cellulose phthalate, hydroxypropylmethyl cellulose acetate succinate, polyvinyl acetaldiethylamino acetate, poly(alkylmethacrylate), poly(vinyl acetate), polymers derived from acrylic or methacrylic acid and their respective esters, and copolymers derived from acrylic or methacrylic acid and their respective esters.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anhydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” and U.S. Pat. No. 6,432,381 for “Methods for targeting drug delivery to the upper and/or lower gastrointestinal tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Certain nanoformulations can enhance the absorption of drugs by releasing drug into the lumen in a controlled manner, thus reducing solubility issues. The intestinal wall is designed to absorb nutrients and to act as a barrier to pathogens and macromolecules. Small amphipathic and lipophilic molecules can be absorbed by partitioning into the lipid bilayers and crossing the intestinal epithelial cells by passive diffusion, while nanoformulation absorption may be more complicated because of the intrinsic nature of the intestinal wall. The first physical obstacle to nanoparticle oral absorption is the mucus barrier which covers the luminal surface of the intestine and colon. The mucus barrier contains distinct layers and is composed mainly of heavily glycosylated proteins called mucins, which have the potential to block the absorption of certain nanoformulations. Modifications can be made to produce nanoformulations with increased mucus-penetrating properties (Ensign et al., “Mucus penetrating nanoparticles: biophysical tool and method of drug and gene delivery,” Adv Mater 24: 3887-3894 (2012)).

Once the mucus coating has been traversed, the transport of nanoformulations across intestinal epithelial cells can be regulated by several steps, including cell surface binding, endocytosis, intracellular trafficking and exocytosis, resulting in transcytosis (transport across the interior of a cell) with the potential involvement of multiple subcellular structures. Moreover, nanoformulations can also travel between cells through opened tight junctions, defined as paracytosis. Non-phagocytic pathways, which involve clathrin-mediated and caveolae-mediated endocytosis and macropinocytosis, are the most common mechanisms of nanoformulation absorption by the oral route.

Non-oral administration can provide various benefits, such as direct targeting to the desired site of action and an extended period of drug action. Transdermal administration has been optimized for nanoformulations, such as solid lipid nanoparticles (SLNs) and NEs, which are characterized by good biocompatibility, lower cytotoxicity and desirable drug release modulation (Cappel and Kreuter, “Effect of nanoparticles on transdermal drug delivery. J Microencapsul 8: 369-374 (1991)). Nasal administration of nanoformulations allows them to penetrate the nasal mucosal membrane, via a transmucosal route by endocytosis or via a carrier- or receptor-mediated transport process (Illum, “Nanoparticulate systems for nasal delivery of drugs: a real improvement over simple systems?” J. Pharm. Sci 96: 473-483 (2007)), an example of which is the nasal administration of chitosan nanoparticles of tizanidine to increase brain penetration and drug efficacy in mice (Patel et al., “Improved transnasal transport and brain uptake of tizanidine HCl-loaded thiolated chitosan nanoparticles for alleviation of pain,” J. Pharm. Sci 101: 690-706 (2012)). Pulmonary administration provides a large surface area and relative ease of access. The mucus barrier, metabolic enzymes in the tracheobronchial region and macrophages in the alveoli are typically the main barriers for drug penetration. Particle size is a major factor determining the diffusion of nanoformulation in the bronchial tree, with particles in the nano-sized region more likely to reach the alveolar region and particles with diameters between 1 and 5 μm expected to deposit in the bronchioles (Musante et al., “Factors affecting the deposition of inhaled porous drug particles,” J Pharm Sci 91: 1590-1600 (2002)). A limit to absorption has been shown for larger particles, presumably because of an inability to cross the air-blood barrier. Particles can gradually release the drug, which can consequently penetrate into the blood stream or, alternatively, particles can be phagocytosed by alveolar macrophages (Bailey and Berkland, “Nanoparticle formulations in pulmonary drug delivery,” Med. Res. Rev., 29: 196-212 (2009)).

Certain nanoformulations have a minimal penetration through biological membranes in sites of absorption and for these, i.v. administration can be the preferred route to obtain an efficient distribution in the body (Wacker, “Nanocarriers for intravenous injection—The long hard road to the market,” Int. J. Pharm., 457: 50-62., 2013).

The distribution of nanoformulations can vary widely depending on the delivery system used, the characteristics of the nanoformulation, the variability between individuals, and the rate of drug loss from the nanoformulations. Certain nanoparticles, such as solid drug nanoparticles (SDNs), improve drug absorption, which does not require them to arrive intact in the systemic circulation. Other nanoparticles survive the absorption process, thus altering the distribution and clearance of the contained drug.

Nanoformulations of a certain size and composition can diffuse in tissues through well-characterized processes, such as the enhanced permeability and retention effect, whereas others accumulate in specific cell populations, which allows one to target specific organs. Complex biological barriers can protect organs from exogenous compounds, and the blood-brain barrier (BBB) represents an obstacle for many therapeutic agents. Many different types of cells including endothelial cells, microglia, pericytes and astrocytes are present in the BBB, which exhibits extremely restrictive tight junctions, along with highly active efflux mechanisms, limiting the permeation of most drugs. Transport through the BBB is typically restricted to small lipophilic molecules and nutrients that are carried by specific transporters. One of the most important mechanisms regulating diffusion of nanoformulations into the brain is endocytosis by brain capillary endothelial cells.

Recent studies have correlated particle properties with nanoformulation entry pathways and processing in the human BBB endothelial barrier, indicating that uncoated nanoparticles have limited penetration through the BBB and that surface modification can influence the efficiency and mechanisms of endocytosis (Lee et al., “Targeting rat anti-mouse transferrin receptor monoclonal antibodies through blood-brain barrier in mouse,” J. Pharmacol. Exp. Ther. 292: 1048-1052 (2000)). Accordingly, surface-modified nanoparticles which cross the BBB, and deliver one or more of the compounds described herein, are within the scope of the invention.

Macrophages in the liver are a major pool of the total number of macrophages in the body. Kupffer cells in the liver possess numerous receptors for selective phagocytosis of opsonized particles (receptors for complement proteins and for the fragment crystallizable part of IgG). Phagocytosis can provide a mechanism for targeting the macrophages, and providing local delivery (i.e., delivery inside the macrophages) of the compounds described herein.

Nanoparticles linked to polyethylene glycol (PEG) have minimal interactions with receptors, which inhibits phagocytosis by the mononuclear phagocytic system (Bazile et al., “Stealth Me.PEG-PLA nanoparticles avoid uptake by the mononuclear phagocytes system,” J. Pharm. Sci. 84: 493-498 (1995)).

Representative nanoformulations include inorganic nanoparticles, SDNs, SLNs, NEs, liposomes, polymeric nanoparticles and dendrimers. The compounds described herein can be contained inside a nanoformulation, or, as is sometimes the case with inorganic nanoparticles and dendrimers, attached to the surface. Hybrid nanoformulations, which contain elements of more than one nanoformulation class, can also be used.

SDNs are lipid-free nanoparticles, which can improve the oral bioavailability and exposure of poorly water-soluble drugs (Chan, “Nanodrug particles and nanoformulations for drug delivery,” Adv. Drug. Deliv. Rev. 63: 405 (2011)). SDNs include a drug and a stabilizer, and are produced using ‘top-down’ (high pressure homogenization and wet milling) or bottom-up (solvent evaporation and precipitation) approaches.

SLNs consist of a lipid (or lipids) which is solid at room temperature, an emulsifier and water. Lipids utilized include, but are not limited to, triglycerides, partial glycerides, fatty acids, steroids and waxes. SLNs are most suited for delivering highly lipophilic drugs.

Liquid droplets of less than a 1000 nm dispersed in an immiscible liquid are classified as NEs. NEs are used as carriers for both hydrophobic and hydrophilic agents, and can be administered orally, transdermally, intravenously, intranasally, and ocularly. Oral administration can be preferred for chronic therapy, and NEs can effectively enhance oral bioavailability of small molecules, peptides and proteins.

Polymeric nanoparticles are solid particles typically around 200-800 nm in size, which can include synthetic and/or natural polymers, and can optionally be pegylated to minimize phagocytosis. Polymeric nanoparticles can increase the bioavailability of drugs and other substances, compared with traditional formulations. Their clearance depends on several factors, including the choice of polymers (including polymer size, polymer charge and targeting ligands), with positively charged nanoparticles larger than 100 nm being eliminated predominantly via the liver (Alexis et al., Factors affecting the clearance and biodistribution of polymeric nanoparticles. Mol Pharm 5: 505-515 (2008)).

Dendrimers are tree-like, nanostructured polymers which are commonly 10-20 nm in diameter.

Liposomes are spherical vesicles which include a phospholipid bilayer. A variety of lipids can be utilized, allowing for a degree of control in degradation level. In addition to oral dosing, liposomes can be administered in many ways, including intravenously (McCaskill et al., 2013), transdermally (Pierre and Dos Santos Miranda Costa, 2011), intravitreally (Honda et al., 2013) and through the lung (Chattopadhyay, 2013). Liposomes can be combined with synthetic polymers to form lipid-polymer hybrid nanoparticles, extending their ability to target specific sites in the body. The clearance rate of liposome-encased drugs is determined by both drug release and destruction of liposomes (uptake of liposomes by phagocyte immune cells, aggregation, pH-sensitive breakdown, etc.) (Ishida et al., “Liposome clearance,” Biosci Rep 22: 197-224 (2002)).

One of more of these nanoparticulate formulations can be used to deliver the active agents described herein to the macrophages, across the blood brain barrier, and other locations as appropriate.

### Controlled Release Formulations

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

The terms used in describing the invention are commonly used and known to those skilled in the art. As used herein, the following abbreviations have the indicated meanings:

| | |
|---|---|
| DCM | Dichloromethane |
| DIPEA | N,N-Diisopropylethylamine |
| DME | Dimethoxyethane |
| DMF | Dimethylformamide |
| DMSO | Dimethyl sulfoxide |
| EDCI | N-(3-Dimethylaminopropyl)-N- |
| | ethylcarbodiimide hydrochloride |
| EtOAc | ethyl acetate |
| HATU | 1-[Bis(dimethylamino)methylene]-1H-1,2,3-triazolo |
| | [4,5-b]pyridinium 3-oxidhexafluorophosphate |
| HOBt | Hydroxybenzotriazole |
| MeOH | Methanol |
| THF | tetrahydrofuran |
| X-phos | 2-Dicyclohexylphosphino-2′,4′,6′-triisopropylbiphenyl |

### VIII. General Methods for Preparing Active Compounds

Methods for the facile preparation of active compounds are known in the art and result from the selective combination known methods. The compounds disclosed herein can be prepared as described in detail below, or by other methods known to those skilled in the art. It will be understood by one of ordinary skill in the art that variations of detail can be made without departing from the spirit and in no way limiting the scope of the present invention.

The various reaction schemes are summarized below.
Scheme 1 Synthetic approach to compound 5.
Scheme 2 Alternate synthetic approach to intermediate 4.
Scheme 3 Synthetic approach to compounds 8 and 10.
Scheme 4 An alternate synthetic approach to compound 8 and 10.
Scheme 5 Synthetic approach to compounds of general formula 15.
Scheme 6 Synthetic approach to compounds of general formula 16.
Scheme 7 Synthetic approach to compounds of general formula 17.
Scheme 8 Synthetic approach to compounds of general formula 22.
Scheme 9 Alternative synthetic approach to compounds of general formula 22.

Compounds of general Formula A can be accomplished by one of ordinary skill in the art, using methods outlined in: (a) Wang, L.; Sullivan, G. M.; Hexamer, L. A.; Hasvold, L. A.; Thalji, R.; Przytulinska, M.; Tao, Z. F.; Li, G.; Chen, Z.; Xiao, Z.; Gu, W. Z.; Xue, J.; Bui, M. H.; Merta, P.; Kovar, P.; Bouska, J. J.; Zhang, H.; Park, C.; Stewart, K. D.; Sham, H. L.; Sowin, T. J.; Rosenberg, S. H.; Lin, N. H. J. Med. Chem., 2007, 50 (17), 4162-4176; b) Hasvold L A1, Wang L, Przytulinska M, Xiao Z, Chen Z, Gu W Z, Merta P J, Xue J, Kovar P, Zhang H, Park C, Sowin T J, Rosenberg S H, Lin N H. Bioorg. Med. Chem. Lett. 2008, 18, 2311-2315; c) Giannotti, D.; Viti, G.; Sbraci, P.; Pestellini, V.; Volterra, G.; Borsini, F.; Lecci, A.; Meli, A.; Dapporto, P.; Paoli, P. J. Med. Chem., 1991, 34, 1356-1362; c) Ramirez-Martinez, J. F.; González-Chávez, R.; Guerrero-Alba, R.; Reyes-Gutiérrez, P. E.; Martínez, R.; Miranda-Morales, M.; Espinosa-Luna, R.; González-Chávez, M. M.; Barajas-López, C. Molecules, 2013, 18, 894-913 and by general Schemes 1-9.

In the schemes described herein, if an intermediate includes functional groups that might interfere with, or be decomposed or otherwise converted during certain steps, such functional groups can be protected using suitable protecting groups. After these steps, protected functional groups, if any, can be deprotected.

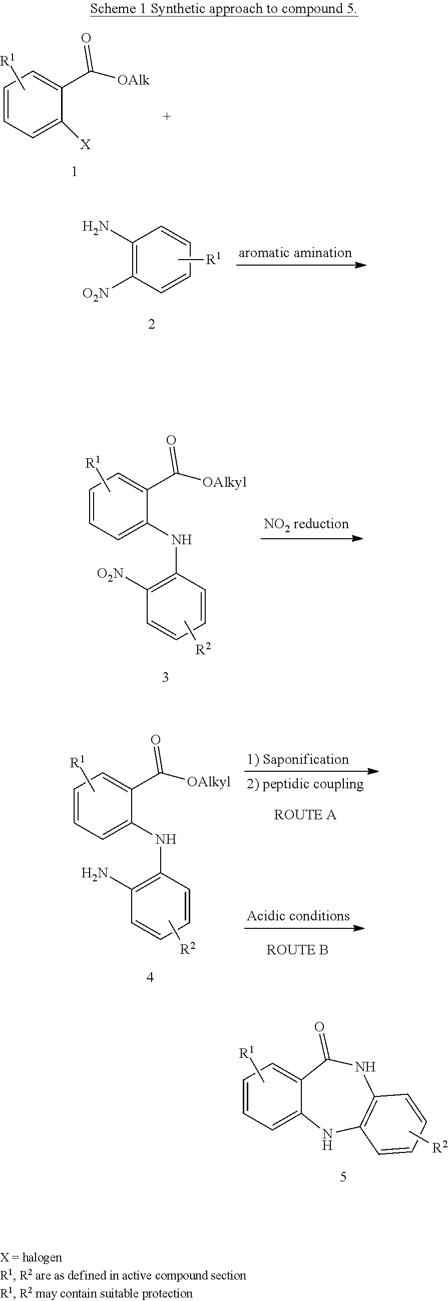

Compound 5 can be obtained, for instance, by the chemistry described in Scheme 1. Reaction of a compound of general formula 1 with an appropriately substituted nitro aniline of general formula 2 in the presence of Cu and an inorganic base such a K₂CO₃, Na₂CO₃ or Cs₂CO₃ can provide intermediate 3. Reduction of the nitro group using for instance Pt/C in the presence of hydrogen in an alcoholic solvent system or SnCl₂ in EtOAc can give compound of general formula 4. Compound 4 can be cyclized in the presence of an acid such as HCl or p-toluene sulfonic acid (Route B). Alternatively, compound 4 can be treated in basic condition with for instance LiOH in a mixture of water and THF to give an acid intermediate which can be then cyclized under classic peptidic conditions using a coupling agent such as HATU in presence of an organic base such as Et₃N (Route A).

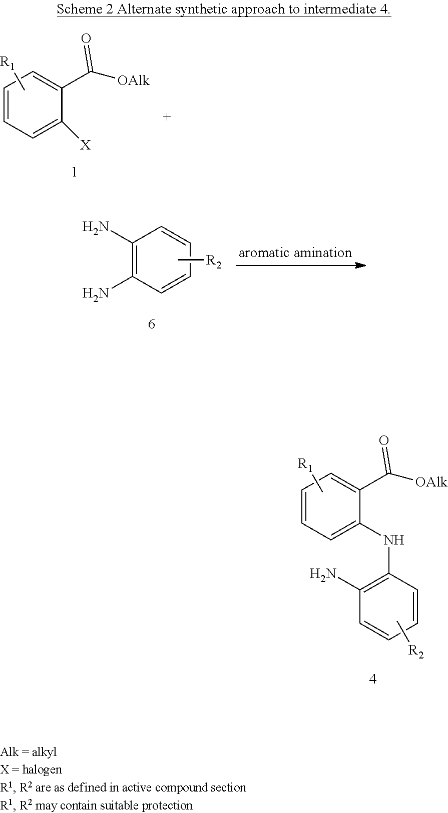

Compounds of general formula 4 can also be prepared by reaction compound of general formula 1 with a diamine of general formula 6 in the presence of Cu and an inorganic base such a K₂CO₃, Na₂CO₃ or Cs₂CO₃.

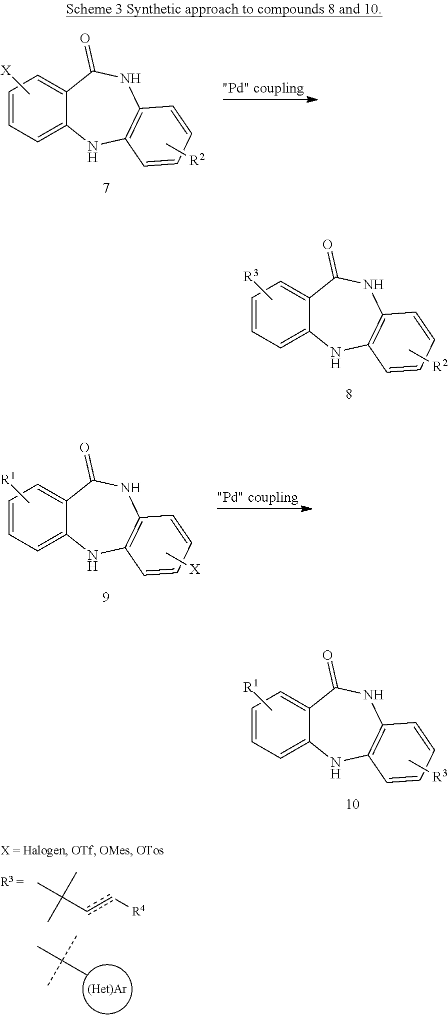

Compounds of general formulas 8 and 10 can be obtained from compounds of general formula 7 or 9 where X is a leaving group such as a halogen, a triflate, a mesylate or a tosylate, by coupling of an alkyne, an alkyl, an alkene, an organoborane or an organostannane derivative under classical palladium catalyzed Sonogashira, Heck, Suzuki or Stille coupling conditions.

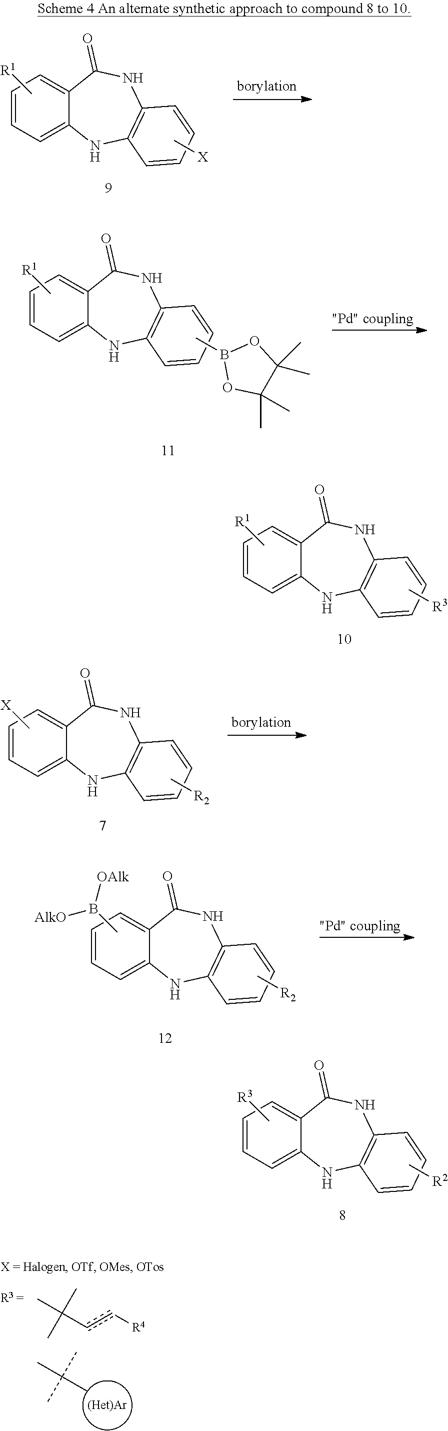

Alternatively, compounds of general formulas 8 and 10 can be prepared by borylation of compounds of general formulas 7 or 9, where X is a leaving group such as a halogen, a triflate, a mesylate or a tosylate. Intermediate 11 and 12 can then be reacted, under classical palladium catalyzed Suzuki coupling conditions, with an aryl, a heteroaryl, an alkene, an alkyne containing a leaving group such as a halogen, a triflate, a mesylate or a tosylate.

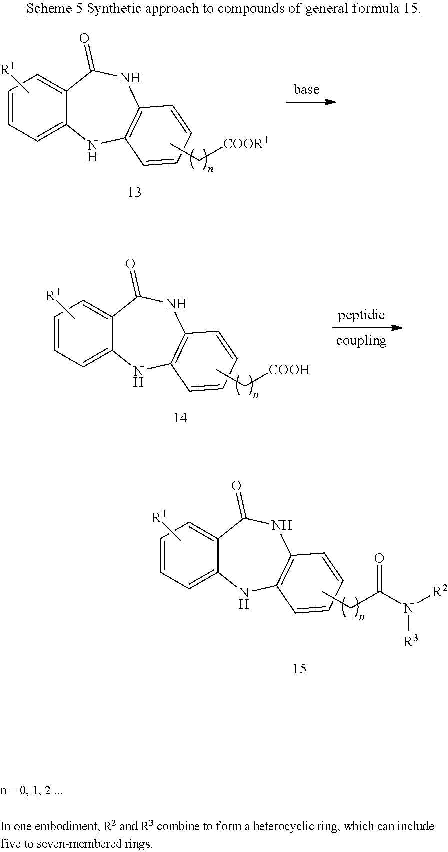

Compounds of general formula 15 can be prepared from esters of general formula, obtained from the chemistry described above, by treatment in basic condition with, for instance, LiOH in a mixture of water and THF to give an acid intermediate which can be then coupled with an amine under classic peptidic conditions using a coupling agent such as HATU in presence of an organic base such as Et₃N.

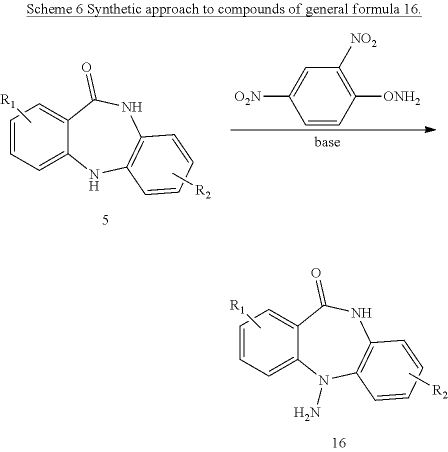

Compounds of general formula 16 can be obtained by treatment with an aminating agent such as O-(2,4-dinitrophenyl)hydroxylamine in presence of a base.

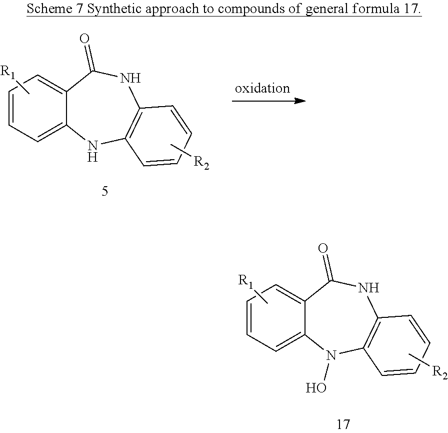

Compounds of general formula 17 can be obtained by treatment with an oxidizing agent such as mCPBA.

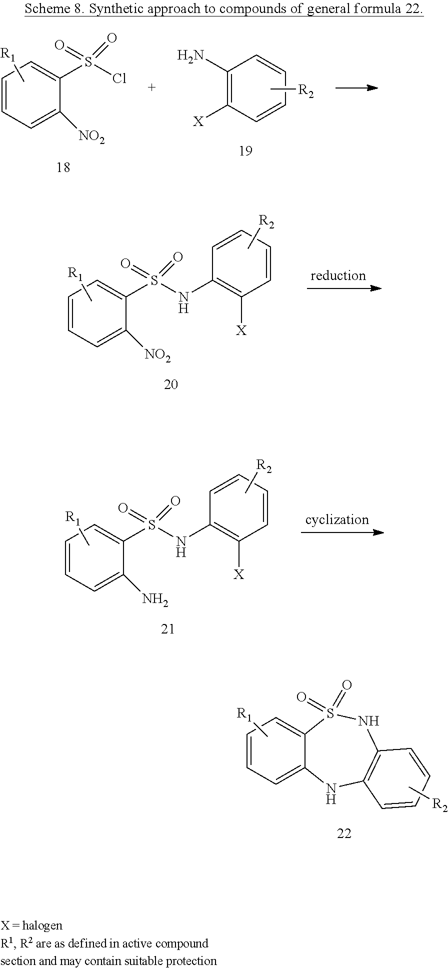

Compounds of general formula 22 can be obtained by the chemistry described in Scheme 8. Reaction of a compound of general formula 18 with an appropriately substituted aniline of general formula 19 in the presence an organic base such as pyridine or trimethylamine can provide intermediate 20. Reduction of the nitro group using for instance Pt/C in the presence of hydrogen in an alcoholic solvent system or SnCl₂ in EtOAc can give compound of general formula 21. Compound 21 can be cyclized in the presence of Cu and an inorganic base such a K₂CO₃, Na₂CO₃ or Cs₂CO₃.

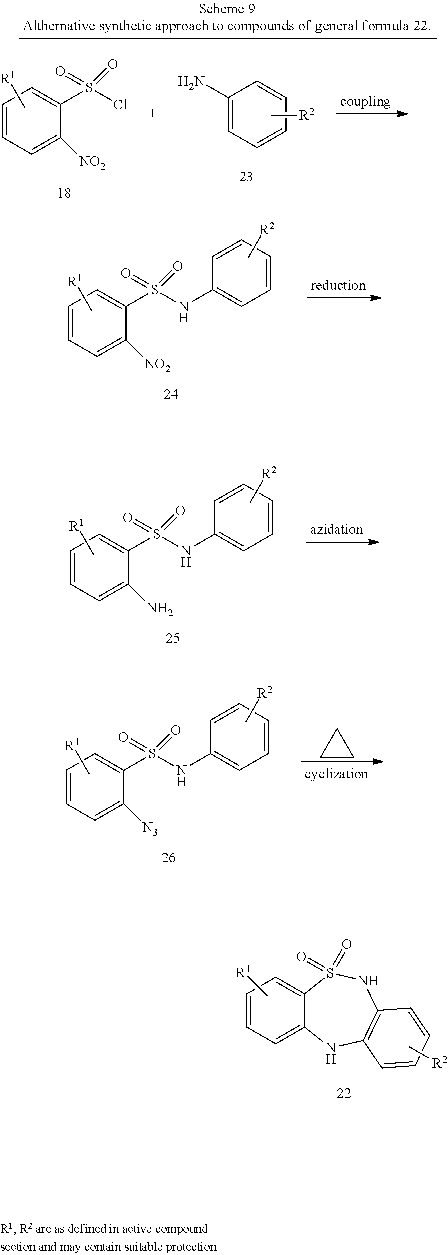

Alternatively, compounds of general formula 22 can be obtained through the chemistry described in Scheme 9. Reaction of a compound of general formula 18 can react with an appropriately substituted aniline of general formula 23 in the presence an organic base such as pyridine or triethylamine to provide intermediate 24. Reduction of the nitro group using for instance Pt/C in the presence of hydrogen in an alcoholic solvent system or SnCl₂ in EtOAc can give compound of general formula 25. Azidation of 25 can be performed using, for instance, NaNO₂, CF₃COOH and NaN₃ to give compounds of general formula 26. Compounds 26 can be cyclized at high temperature in a high boiling point solvents such as dihexyl ether.

### Substitution of Aromatic Rings

In various reaction schemes shown above, the aromatic rings are substituted with various R¹ and R² substituents. It is known in the art how to provide substituents on aromatic rings. For example, where it is desirable to provide substitution on one or both of the aromatic rings, electrophilic aromatic substitution can be used to provide desired functionality. For example, alkyl, aryl, heteroaryl, alkaryl, arylalkyl, alkenyl, alkynyl, and acyl groups can be added using Friedel-Crafts alkylation/arylation/acylation reactions. Other electrophilic aromatic substitution reactions can be used, for example, to provide halogens, such as by forming chloronium or bromonium ions in situ and reacting them with the aromatic ring, or by forming sulfonium or nitronium ions to provide sulfonyl or nitro groups.

Friedel Crafts alkylation is conducted using an appropriate halo-alkyl moiety, and a Lewis acid. The alkyl moiety forms a carbocation, and electrons from the aryl ring form a bond with the carbocation, placing a positive charge on the aryl ring. The aryl ring then loses a proton. Alkyl and alkaryl moieties (such as benzyl moieties) can be added in this fashion.

Friedel Crafts acylation is similar, but uses an acid halide, such as an acid chloride, to place a ketone moiety on the ring. The acid halide can be an alkyl acid, such as acetic acid, propionic acid, butyric acid, and the like, or can be an aromatic acid, such as benzoic acid, p-toluic acid, and the like.

Friedel Crafts arylation (also known as the Scholl reaction) is a coupling reaction with two aryl rings, catalyzed by a Lewis acid. The proton lost during the coupling reaction serves as an additional catalyst. Typical Reagents are iron(III) chloride in dichloromethane, copper(II) chloride, PIFA and boron trifluoride etherate in dichloromethane, Molybdenum(V) chloride and lead tetraacetate with BF₃ in acetonitrile.

Substitution typically occurs at a position ortho or para to the amine groups, and meta to nitro groups. Accordingly, depending on the desired functionality and position, it may be desirable to start with an amine group, and place a substituent So, positions 3, 6, and 8 are typically functionalized using this chemistry. Substitution of the naphthalene ring at a meta position to the amine groups (i.e., positions 2 and 7) can be performed by oxidizing the amine group(s) to nitro groups, which leads to meta substitution. The nitro groups can then be reduced back to the amine groups.

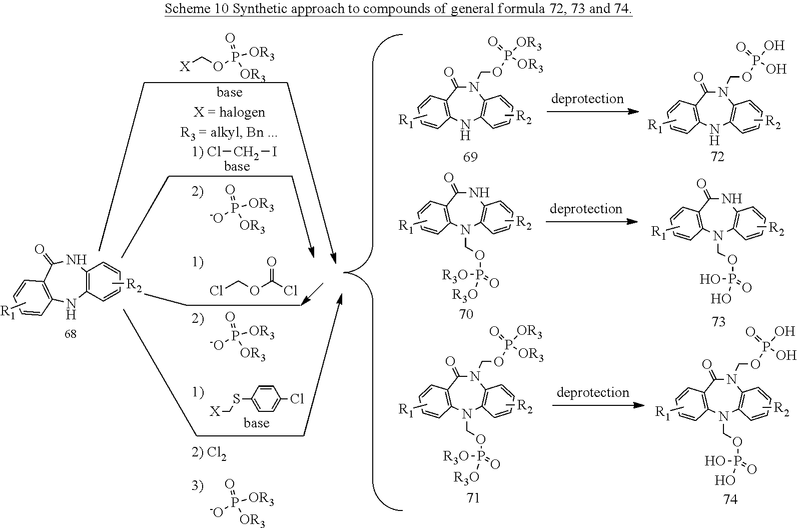

Compounds of general formula 72, 73 and 74. can be obtained by the chemistry described in Scheme 10. Phosphate ester intermediate 69, 70 and 71 can be formed from 68 by reaction with an halogenomethylphosphate ester in presence of a base such as, but not limited to, NaH or Cs₂CO₃. Alternatively, phosphate ester intermediate 69, 70 and 71 can be prepared by reaction of 68 with chloroiodomethane in presence of a base such as, but not limited to, NaH or Cs₂CO₃ followed by reaction with a phosphate diester salt, in which the salt can be, but is not limited to, Na⁺, K⁺ or tetraalkylammonium. Phosphate ester intermediate 69, 70 and 71 can also be made by reacting 68 with first, a (halogenomethyl)(4-chlorophenyl)sulfane and then chlorine followed by substitution of the resulting N-chloromethyl intermediate with a phosphate diester salt. Finally, phosphate ester 69, 70 and 71 can then be deprotectedunder acidic conditions such as, but not limited to, HCl in dioxane, TFA in toluene, AcOH in water and acetonitrile, when R₃=tBu, or by hydrogenation, when R₃=Bn.

### Incorporation of Deuterium:

It is expected that single or multiple replacement of hydrogen with deuterium (carbon-hydrogen bonds to carbon-deuterium bond) at site(s) of metabolism on ROR modulators will slow down the rate of metabolism. This can provide a relatively longer half-life, and slower clearance from the body. The slow metabolism of a therapeutic ROR modulators is expected to add extra advantage to a therapeutic candidate, while other physical or biochemical properties are not affected.

Methods for incorporating deuterium into organic derivatives are well known to those of skill in the art. Representative methods are disclosed in Angew. Chem. Int. Ed. Engl. 2007, 46, 7744-7765. Accordingly, using these techniques, one can provide one or more deuterium atoms in the ROR modulators described herein.

The present invention will be better understood with reference to the following non-limiting examples.

### Example 1

### Synthesis of Compound 1 and Compounds 34-46.

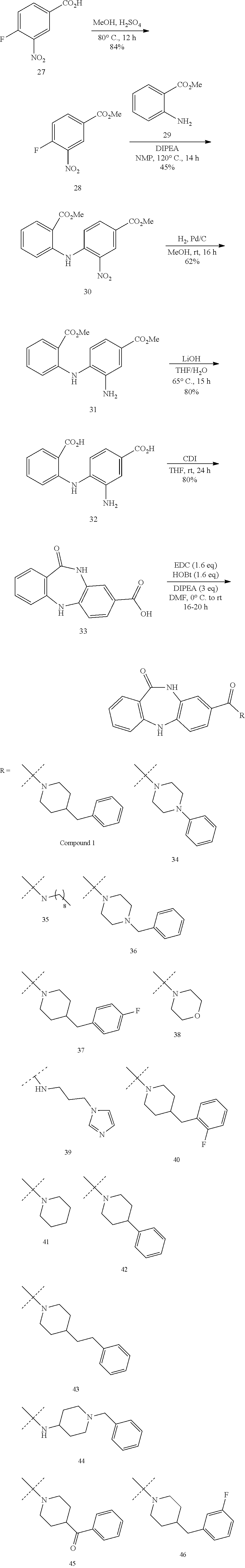

### Methyl 4-fluoro-3-nitrobenzoate (28)

4-Fluoro-3-nitrobenzoic acid 27 (10 g, 54 mmol) was dissolved in methanol (200 mL) and cone. H₂SO₄ (1 mL) at room temperature. The reaction mixture was stirred overnight at 80° C. After completion of the reaction, the solvent was evaporated under reduced pressure. The crude mixture was diluted with H₂O (200 ml) and basified with a saturated solution of NaHCO₃. The precipitated solid was filtered, washed with water (2×100 mL) and dried under vacuum to afford compound 28 as a white solid (10.75 g, 84%); ¹H NMR (400 MHz, DMSO-d₆): δ 8.54 (dd, J=7.3, 2.3 Hz, 1H), 8.31 (ddd, J=8.8, 4.3, 2.3 Hz, 1H), 7.73 (dd, J=11.1, 8.7 Hz, 1H), 3.90 (s, 3H); ¹³C NMR (101 MHz, DMSO-d₆): 158.7, 156.0, 136.9, 136.8 (d, J=10.8 Hz), 127.1 (d, J=1.6 Hz), 126.7 (d, J=3.9 Hz), 119.4 (d, J=21.7 Hz), 52.9; ¹⁹F NMR (377 MHz, DMSO) δ −111.97.

### Methyl 4-((2-(methoxycarbonyl)phenyl)amino)-3-nitrobenzoate (30)

To a solution of methyl 4-fluoro-3-nitrobenzoate 28 (1 g, 6.61 mmol) in NMP (20 mL) were added DIPEA (0.76 ml, 19.83 mmol) and methyl 4-fluoro-3-nitrobenzoate 29 (1.5 g, 9.92 mmol) at room temperature, under inert atmosphere. The mixture was stirred at 120° C. for 14 h and after completion of the reaction, the mixture was cooled down to room temperature, diluted with diethyl ether (20 ml) and stirred for 1 h, The obtained solid was filtered, washed with EtOAc (20 mL) and dried under vacuum to afford compound 30 as a yellow solid (996 mg, 45%); ¹H NMR (400 MHz, DMSO-d₆): δ 11.15 (s, 1H), 8.66 (d, J=2.1 Hz, 1H), 8.09-7.96 (m, 2H), 7.71-7.61 (m, 2H), 7.59 (d, J=9.0 Hz, 1H), 7.29 (ddd, J=8.2, 5.9, 2.1 Hz, 1H), 3.87 (s, 6H); ¹³C NMR (101 MHz, DMSO): δ 166.6, 164.5, 142.7, 139.7, 135.4, 134.7, 133.9, 131.5, 128.0, 124.2, 121.9, 120.4, 120.0, 117.8, 52.5, 52.3.

### Methyl 3-amino-4-((2-(methoxycarbonyl)phenyl)amino)benzoate (31)

A solution of methyl 4-((2-(methoxycarbonyl)phenyl)amino)-3-nitrobenzoate 30 (2.5 g, 7.5 mmol) and 10% Pd/C (1.25 g, 50% wet) in MeOH was stirred under hydrogen atmosphere for 16 h at room temperature. After completion of the reaction, the mixture was filtered through Celite and washed with 20% MeOH/DCM (250 mL). The filtrate was concentrated and the crude residue was purified was purified by flash column chromatography (AcOEt/hexanes 3/7) to afford compound 31 as a yellow solid (1.4 g, 62%); ¹H NMR (400 MHz, DMSO-d₆) δ 8.93 (s, 1H), 7.91 (d, J=8.4 Hz, 1H), 7.52-7.36 (m, 2H), 7.21 (s, 2H), 6.95 (d, J=8.4 Hz, 1H), 6.88-6.72 (m, 1H), 5.19 (s, 2H), 3.85 (s, 3H), 3.80 (s, 3H); ¹³C NMR (101 MHz, DMSO) δ 167.9, 166.3, 146.7, 142.2, 134.3, 131.2, 130.6, 125.5, 121.9, 118.1, 117.7, 116.2, 114.9, 112.3, 51.9, 51.8.

### 3-Amino-4-((2-carboxyphenyl)amino)benzoic acid (32)

To a solution of methyl 3-amino-4-((2-(methoxycarbonyl)phenyl)amino)benzoate 31 (1.4 g, 4.66 mmol) in a mixture of THF:H₂O (2.5/1, 105 ml) was added lithium hydroxide monohydrate (1.75 g, 41.9 mmol) at room temperature. The reaction mixture was stirred at 65° C. for 5 h and the volatiles were removed under vacuum. The pH of the residue was acidified to 4 with 2N HCl. The precipitated solid was filtered, washed with water (10 ml) and dried under vacuum to afford compound 32 as a white solid (1 g, 80%). ¹H NMR (400 MHz, DMSO-d₆) δ 12.64 (s, 2H), 9.20 (s, 1H), 7.89 (d, J=7.9 Hz, 1H), 7.44-7.31 (m, 2H), 7.18 (s, 2H), 6.91 (d, J=8.5 Hz, 1H), 6.76 (t, J=7.5 Hz, 1H), 5.06 (s, 2H); ¹³C NMR (101 MHz, DMSO) δ 169.8, 167.4, 147.3, 142.1, 134.0, 131.7, 130.4, 126.6, 121.9, 118.4, 117.3, 116.5, 114.5, 112.8.

### 11-oxo-10,11-Dihydro-5H-dibenzo[b,e][1,4]diazepine-8-carboxylic acid (33)

A solution of compound 32 (1 g, 3.67 mmol) and CDI (2.39 g, 14.6 mmol) in THF (40 mL) was stirred at room temperature for 24 h under inert atmosphere. After completion of the reaction, the volatiles were removed under vacuum. The pH of the residue was adjusted to 2 using 2N HCl. The precipitated solid was filtered, washed with pentane (10 mL) and dried under vacuum to afford compound 33 as a pale green solid (746 mg, 80%). ¹H NMR (400 MHz, DMSO-d₆) 12.66 (s, 1H), 9.93 (s, 1H), 8.28 (s, 1H), 7.70 (dd, J=7.9, 1.7 Hz, 1H), 7.57-7.49 (m, 2H), 7.36 (td, J=7.9, 1.7 Hz, 1H), 7.02 (dd, J=17.0, 8.1 Hz, 2H), 6.91 (t, J=7.4 Hz, 1H); ¹³C NMR (101 MHz, DMSO) δ 167.4, 166.7, 148.8, 143.8, 133.5, 132.3, 129.2, 126.0, 125.0, 122.5, 122.2, 121.1, 119.4, 119.2, SM (IS): m/z: 255.4 [M+1]

### General Procedure I

To a solution of compound 33 (100 mg, 0.393 mmol) in DMF (5 ml) were added EDCI, HCl (121 mg, 0.629 mmol), HOBt (85 mg, 6.29 mmol), amine (0.511 mmol) and DIPEA (205 ml, 0.117 mmol) at 0° C. under inert atmosphere. The reaction mixture was then stirred at room temperature for 16-24 h. After completion of the reaction, water was added. The crude solid was filtered and washed with water. The crude residue was purified by chromatography on silica gel (DCM/Methanol) to afford the desired compound.

### 8-(4-benzylpiperidine-1-carbonyl)-10,11a-dihydro-4aH-dibenzo[b,e][1,4]diazepin-11(5H)-one (Compound 1)

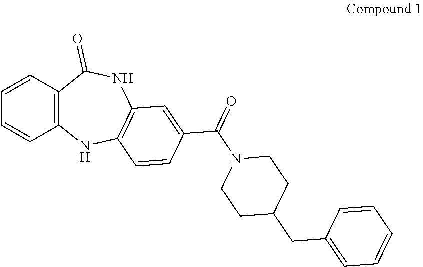 [SMILES: O=C1Nc2cc(C(=O)N3CCC(Cc4ccccc4)CC3)ccc2Nc2ccccc21]

Compound 1 was prepared from 4-benzylpiperidine (91 ml, 0.511 mmol) following general procedure I. Column chromatography: DCM/Methanol (95:5); Light yellow solid (111 mg, 68%); ¹H NMR (400 MHz, DMSO-d₆) δ 9.91 (s, 1H), 8.06 (s, 1H), 7.68 (d, J=7.8 Hz, 1H), 7.34 (t, J=7.8 Hz, 1H), 7.27 (t, J=7.8 Hz, 2H), 7.16 (d, J=7.8 Hz, 3H), 7.05-6.99 (m, 4H), 6.89 (d, J=7.8 Hz, 1H), 4.34 (s, 1H), 3.67 (s, 1H), 2.78 (s, 2H), 2.52 (s, 2H), 1.75 (s, 1H), 1.56 (s, 2H), 1.17-1.02 (m, 2H); ¹³C NMR (101 MHz, DMSO-d₆) δ 168.2, 167.6, 149.6, 140.7, 140.0, 133.4, 132.2, 130.6, 129.3, 129.0, 128.1, 125.8, 123.4, 122.5, 120.9, 120.0, 119.4, 119.1, 42.1, 37.5, 31.6; SM (IS): 412.5 m/z: [M+1]; HRMS (ESI) [M+H]⁺ calcd for C₂₆H₂₆N₃O₂: 412.1947, found: 412.2018.

### 8-(4-phenylpiperazine-1-carbonyl)-5H-dibenzo[b,e][1,4]diazepin-11(10H)-one (34)

Compound 34 was prepared from 4-phenylpiperazine (77 ml, 0.511 mmol) following general procedure I. Column chromatography: DCM/Methanol (95:5); Beige solid (100 mg, 64%); ¹H NMR (400 MHz, DMSO-d₆) δ 9.94 (s, 1H), 8.12 (s, 1H), 7.70 (d, J=7.7 Hz, 1H), 7.37 (t, J=7.6 Hz, 1H), 7.23 (t, J=7.9 Hz, 2H), 7.06 (d, J=2.6 Hz, 3H), 7.01 (d, J=8.0 Hz, 1H), 6.97-6.93 (m, 3H), 6.82 (t, J=7.2 Hz, 1H), 3.72-3.52 (m, 4H), 3.15 (s, 4H); ¹³C NMR (101 MHz, DMSO-d₆) δ 168.4, 167.6, 150.8, 149.5, 141.0, 133.4, 132.2, 129.8, 129.4, 129.0, 123.8, 122.4, 120.9, 120.4, 119.4, 119.4, 119.1, 115.9, 48.2; SM (IS): 399.5 m/z: [M+1]; HRMS (ESI) [M+H]⁺ calcd for C₂₄H₂₃N₄O₂: 399.1810, found: 399.1812

### N-nonyl-11-oxo-10,11-dihydro-5H-dibenzo[b,e][1,4]diazepine-8-carboxamide (35)

Compound 35 was prepared from nonylamine (93 μl, 0.511 mmol) following general procedure I. The reaction mixture was quenched with water and extracted with ethyl acetate (3×5 mL). The combined organic layers were dried over magnesium sulfate and concentrated under reduced pressure and finally purified by column chromatography eluting with DCM/Methanol (99:1 to 95/5); yellow solid (70 mg, 47%); ¹H NMR (400 MHz, DMSO-d₆) δ 9.91 (s, 1H), 8.24 (t, J=5.4 Hz, 1H), 8.13 (s, 1H), 7.69 (dd, J=7.9, 1.6 Hz, 1H), 7.47-7.39 (m, 2H), 7.39-7.32 (m, 1H), 7.00 (dd, J=7.9, 5.4 Hz, 2H), 6.91 (t, J=7.9 Hz, 1H), 3.20 (q, J=6.6 Hz, 2H), 1.49 (d, J=8.0 Hz, 2H), 1.30-1.22 (m, 12H), 0.85 (t, J=6.6 Hz, 3H); ¹³C NMR (101 MHz, DMSO-d₆) δ 167.5, 165.3, 149.4, 142.4, 133.4, 132.2, 129.5, 129.2, 123.2, 122.4, 121.0, 120.9, 119.1, 119.0, 31.3, 29.1, 29.0, 28.8, 28.7, 26.5, 22.1, 14.0; SM (IS): 380.2 m/z: [M+1]; HRMS (ESI) [M+H]⁺ calcd for C₂₃H₃₀N₃O₂: 380.2327, found: 380.2332.

### 8-(4-benzylpiperazine-1-carbonyl)-5H-dibenzo[b,e][1,4]diazepin-11(10H)-one. (36)

Compound 36 was prepared from 1-benzylpiperazine (89 ml, 0.511 mmol) following general procedure I. Column chromatography: DCM/Methanol (99:1 to 95/5); yellow solid (42 mg, 26%); ¹H NMR (400 MHz, DMSO-d₆) 9.92 (s, 1H), 8.09 (s, 1H), 7.69 (dd, J=7.9, 1.7 Hz, 1H), 7.40-7.27 (m, 5H), 7.25 (td, J=5.9, 2.5 Hz, 1H), 7.08-6.93 (m, 4H), 6.91 (t, J=7.9 Hz, 1H), 3.50 (s, 4H), 3.36 (s, 2H), 2.36 (s, 4H); ¹³C NMR (101 MHz, DMSO-d₆) 168.3, 167.6, 149.5, 141.0, 137.8, 133.4, 132.2, 130.0, 129.3, 128.9, 128.2, 127.0, 123.7, 122.4, 120.9, 120.3, 119.4, 119.1, 61.9, 52.6; SM (IS): 413.5 m/z: [M+1]; HRMS (ESI) [M+H]⁺ calcd for C₂₅H₂₅N₄O₂:413.1899, found: 413.1970.

### 8-(4-(4-fluorobenzyl)piperidine-1-carbonyl)-5H-dibenzo[b,e][1,4]diazepin-11(10H)-on (37)

Compound 37 was prepared from 4-(4-fluorobenzyl)piperidine (99 mg, 0.511 mmol) following general procedure I. Column chromatography: DCM:MeOH (99/1 to 95/5); Yellow solid (74 mg, 43%); ¹H NMR (400 MHz, DMSO-d₆) δ 9.92 (s, 1H), 8.07 (s, 1H), 7.69 (dd, J=8.0, 1.7 Hz, 1H), 7.36 (ddd, J=8.0, 7.2, 1.7 Hz, 1H), 7.24-7.19 (m, 2H), 7.13-7.07 (m, 2H), 7.04-6.95 (m, 4H), 6.91 (ddd, J=8.0, 7.2, 1.7 Hz, 1H), 4.36 (s, 1H), 3.68 (s, 1H), 2.86 (s, 2H), 2.52 (s, 2H), 1.75 (s, 1H), 1.56 (s, 2H), 1.11 (qd, J=12.3, 4.2 Hz, 2H). ¹³C NMR (101 MHz, DMSO-d₆): δ 133.4, 132.2, 130.7 (d, J=7.8 Hz), 123.4, 120.9, 120.0, 119.3, 119.1, 114.8 (d, J=20.9 Hz), 41.1, 37.5. ¹⁹F NMR (377 MHz, DMSO-d₆): δ −117.51 (s). SM (IS): 430.5 m/z: [M+1]; HRMS (ESI) [M+H]⁺ calcd for C₂₆H₂₅FN₃O₂: 430.1853, found: 430.1926.

### 8-(morpholine-4-carbonyl)-5,10-dihydro-11H-dibenzo[b,e][1,4]diazepin-11-one (38)

Compound 38 was prepared from morpholine (44 ml, 0.511 mmol) following general procedure I. After 16 h, the reaction was quenched with water and extracted with ethyl acetate (3×5 mL). The combined organic layers were dried over magnesium sulfate and concentrated under reduced pressure, purified by column chromatography eluting with DCM/Methanol (99:1 to 95/5); yellow solid (54 mg, 43%); ¹H NMR (400 MHz, DMSO-d₆) δ 9.92 (s, 1H), 8.10 (s, 1H), 7.70 (dd, J=7.9, 1.6 Hz, 1H), 7.36 (ddd, J=8.6, 7.9, 1.7 Hz, 1H), 7.06-6.94 (m, 4H), 6.96-6.87 (m, 1H), 3.58 (d, J=5.0 Hz, 4H), 3.47 (s, 4H); ¹³C NMR (101 MHz, DMSO-d₆): δ 168.5, 167.6, 149.5, 141.1, 133.4, 132.2, 129.6, 129.4, 123.8, 122.4, 120.9, 120.5, 119.4, 119.1, 66.2, 40.1. SM (IS): 324.5 m/z: [M+1]; HRMS (ESI) [M+H]⁺ calcd for C₁₈H₁₈N₃O: 324.1270, found: 324.1341

### N-(3-(1H-imidazol-1-yl)propyl)-11-oxo-10,11-dihydro-5H-dibenzo[b,e][1,4]diazepine-8-carboxamide (39)

Compound 39 was prepared from 3-(1H-imidazol-1-yl)propan-1-amine (0.511 mmol) following general procedure I. After 16 h, 30 ml of water were added, and the mixture was extracted two times with 20 ml of ethyl acetate. The combined organic layers were dried with MgSO₄, filtered and evaporated under vacuum. The crude solid was purified by column chromatography eluting with DCM/Methanol (99:1 to 95/5); yellow solid (40 mg, 28%). ¹H NMR (400 MHz, DMSO-d₆) δ 9.92 (s, 1H), 8.34 (t, J=5.6 Hz, 1H), 8.15 (s, 1H), 7.70 (dd, J=7.9, 1.7 Hz, 1H), 7.65 (d, J=1.3 Hz, 1H), 7.53-7.41 (m, 2H), 7.36 (ddd, J=8.7, 7.2, 1.7 Hz, 1H), 7.21 (d, J=1.3 Hz, 1H), 7.08-6.97 (m, 2H), 6.93-6.87 (m, 2H), 4.00 (t, J=6.9 Hz, 2H), 3.19 (q, J=6.9 Hz, 2H), 1.93 (p, J=6.9 Hz, 2H), ¹³C NMR (101 MHz, DMSO-d₆): δ 167.5, 165.7, 149.3, 142.5, 137.3, 133.4, 132.2, 129.3, 129.2, 128.4, 123.3, 122.4, 121.0, 121.0, 119.3, 119.1, 119.0, 43.7, 36.4, 30.8. SM (IS): 362.4 m/z: [M+1]; HRMS (ESI) [M+H]⁺ calcd for C₂₀H₂₀N₅O₂: 362.1539, found: 362.1609

### 8-(4-(2-fluorobenzyl)piperidine-1-carbonyl)-5,10-dihydro-11H dibenzo[b,e][1,4]diazepin-11-one (40)

Compound 40 was prepared from 4-(2-fluorobenzyl)piperidine (99 mg, 0.511 mmol) following general procedure I. Column chromatography: DCM:MeOH (99/1 to 95/5); Yellow solid (135 mg, 80%); ¹H NMR (400 MHz, DMSO-d₆): δ 9.92 (s, 1H), 8.08 (s, 1H), 7.70 (d, J=7.8 Hz, 1H), 7.36 (t, J=7.6 Hz, 1H), 7.27 (d, J=8.6 Hz, 2H), 7.14 (q, J=7.3 Hz, 2H), 7.00 (t, J=5.9 Hz, 4H), 6.92 (t, J=7.4 Hz, 1H), 4.35 (s, 1H), 3.69 (s, 1H), 2.75 (s, 2H), 2.58 (d, J=7.0 Hz, 2H), 1.79 (s, 1H), 1.58 (s, 2H), 1.15 (q, J=12.5 Hz, 2H); ¹³C NMR (101 MHz, DMSO-d₆) δ 168.3, 167.6, 161.8, 159.4, 149.6, 140.8, 133.4, 132.2, 131.7 (d, J=5.1 Hz), 130.6, 129.3, 128.1 (d, J=8.3 Hz), 126.6 (d, J=15.9 Hz), 124.2 (d, J=3.3 Hz), 123.4, 122.5, 120.9, 120.0, 119.4, 119.1, 115.1 (d, J=22.3 Hz), 36.6, 35.0, 31.7. ¹⁹F NMR (377 MHz, DMSO-d₆) δ −118.26, SM (IS): 430.1 m/z: [M+1]; HRMS (ESI) [M+H]⁺ calcd for C₂₆H₂₄FN₃O₂: 430.1853, found: 430.1927

### 8-(piperidine-1-carbonyl)-5,10-dihydro-11H-dibenzo[b,e][1,4]diazepin-11-one (41)

Compound 41 was prepared from piperidine (58 ml, 0.511 mmol) following general procedure I. After 24 h, 30 ml of water were added, and the mixture was extracted two times with 20 ml of ethyl acetate. The combined organic layers were dried with MgSO₄, filtered and concentrated under vacuum. The crude solid was purified by column chromatography eluting with DCM/Methanol (99:1 to 95/5); yellow solid (20 mg, 16%); ¹H NMR (400 MHz, DMSO-d₆) δ 9.92 (s, 1H), 8.07 (s, 1H), 7.69 (dd, J=7.9, 1.7 Hz, 1H), 7.40-7.33 (m, 1H), 7.05-6.96 (m, 4H), 6.95-6.88 (m, 1H), 3.42-3.31 (m, 4H), 1.60 (q, J=5.6 Hz, 2H), 1.54-1.41 (m, 4H). ¹³C NMR (101 MHz, DMSO) δ 168.2, 167.7, 149.6, 140.7, 133.4, 132.2, 130.7, 129.4, 123.4, 122.5, 120.9, 120.0, 119.4, 119.1, 25.7, 24.7; HRMS (ESI) [M+H]⁺ calcd for C₁₉H₂₀N₃O₂: 322.1477, found: 322.1548.

### 8-(4-phenylpiperidine-1-carbonyl)-5,10-dihydro-11H-dibenzo[b,e][1,4]diazepin-11-one (42)

Compound 42 was prepared from 4-phenylpiperidine (82 mg, 0.511 mmol) by following general procedure I. Colum chromatography: DCM:MeOH (99/1 to 95/5); yellow solid (67 mg, 43%); ¹H NMR (400 MHz, DMSO-d₆) δ 9.94 (s, 1H), 8.09 (s, 1H), 7.70 (dd, J=7.9, 1.7 Hz, 1H), 7.38-7.32 (m, 1H), 7.32-7.26 (m, 4H), 7.21 (d, J=7.0 Hz, 1H), 7.08-7.02 (m, 3H), 7.00 (dd, J=8.1, 1.1 Hz, 1H), 6.94-6.88 (m, 1H), 4.71-4.39 (m, 1H), 3.95-3.66 (m, 1H), 3.11-2.98 (s, 1H), 2.80 (t, J=12.0 Hz, 2H), 1.98-1.73 (m, 2H), 1.61 (td, J=12.6, 4.1 Hz, 2H). SM (IS): 398.2 m/z: [M+1].

### 8-(4-phenethylpiperidine-1-carbonyl)-5,10-dihydro-11H-dibenzo[b,e][1,4]diazepin-11-one (43)

Compound 43 was prepared from 4-phenethylpiperidine (96 mg, 0.511 mmol) by following general procedure I. Column chromatography: DCM/Methanol (99/1 to 95/5); yellow solid (67 mg, 40%); ¹H NMR (400 MHz, DMSO-d₆ 9.92 (s, 1H), 8.07 (s, 1H), 7.69 (dd, J=7.9, 1.7 Hz, 1H), 7.36 (ddd, J=8.5, 7.9, 1.7 Hz, 1H), 7.28 (t, J=7.9 Hz, 2H), 7.23-7.14 (m, 3H), 7.03-6.97 (d, J=7.0 Hz, 4H), 6.95-6.89 (m, 1H), 4.38 (s, 1H), 3.69 (s, 1H), 2.90 (s, 2H), 2.60 (t, J=7.5 Hz, 3H), 1.73 (s, 2H), 1.52 (d, J=7.4 Hz, 3H), 1.22-0.99 (m, H). ¹³C NMR (101 MHz, DMSO-d₆) 168.6, 168.11, 150.0, 142.7, 141.2, 133.9, 132.6, 131.1, 129.8, 128.7, 128.7, 126.1, 123.8, 122.9, 121.4, 120.5, 119.8, 119.6, 4, 38.3, 35.4, 32.6. SM (IS): 426.2 m/z: [M+1].

### N-(1-benzylpiperidin-4-yl)-11-oxo-10,11-dihydro-5H-dibenzo[b,e][1,4]diazepine-8-carboxamide (44)

Compound 44 was prepared from 1-benzyl-4-aminopiperidine (104 ml, 0.511 mmol) by following general procedure I. Column chromatography: DCM/Methanol (99/1 to 95/5); yellow solid (60 mg, 35%); ¹H NMR (400 MHz, DMSO-d₆) 9.89 (s, 1H), 8.14 (s, 1H), 8.06 (d, J=7.6 Hz, 1H), 7.69 (dd, J=7.9, 1.6 Hz, 1H), 7.45 (d, J=7.0 Hz, 2H), 7.39-7.28 (m, 5H), 7.27-7.23 (m, 1H), 7.00 (t, J=7.9 Hz, 1H), 6.95-6.85 (m, 1H), 3.73 (d, J=7.0 Hz, 1H), 3.46 (s, 2H), 2.81 (d, J=9.8 Hz, 2H), 2.01 (s, 2H), 1.75 (d, J=12.4 Hz, 2H), 1.56 (td, J=11.8, 3.6 Hz, 2H); ¹³C NMR (101 MHz, DMSO-d₆): 168.0, 165.3, 149.8, 142.9, 139.1, 133.8, 132.7, 129.9, 129.6, 129.2, 128.6, 127.3, 123.9, 122.8, 121.5, 121.4, 119.6, 119.4, 62.6, 52.7, 47.3, 32.0. SM (IS): 427.3 m/z: [M+1].

### 8-(4-benzoylpiperidine-1-carbonyl)-5,10-dihydro-11H-dibenzo[b,e][1,4]diazepin-11-one (45)

Compound 45 was prepared from 4-benzoylpiperidine (89 ml, 0.511 mmol) by following general procedure I. Column chromatography: DCM/Methanol (99/1 to 95/5); yellow solid (114 mg, 68%); ¹H NMR (400 MHz, DMSO-d₆) 9.93 (s, 1H), 8.09 (s, 1H), 8.01 (d, J=7.7 Hz, 2H), 7.74-7.61 (m, 2H), 7.55 (t, J=7.5 Hz, 2H), 7.42-7.31 (m, 1H), 7.02 (d, J=7.4 Hz, 4H), 6.91 (t, J=7.5 Hz, 1H), 4.40 (s, 1H), 3.86-3.65 (m, 1H), 3.08 (s, 4H), 1.82 (s, 2H), 1.52 (d, J=13.8 Hz, 3H), ¹³C NMR (101 MHz, DMSO-d₆): δ 201.7, 168.4, 167.6, 149.5, 140.8, 135.4, 133.4, 133.26, 132.2, 130.3, 129.3, 128.8, 128.2, 123.5, 122.4, 120.9, 120.0, 119.4, 119.1, 42.4, 28.5; SM (IS): 426.2 m/z: [M+1].

### 8-(4-(3-fluorobenzyl)piperidine-1-carbonyl)-5,10-dihydro-11H-dibenzo[b,e][1,4]diazepin-11-one (46)

Compound 46 was prepared following general procedure I with 4-(3-fluorobenzyl)piperidine hydrochloride (115 mg, 0.511 mmol). Column chromatography: DCM:MeOH (99/1 to 95/5); Yellow solid (90 mg, 53%); ¹H NMR (400 MHz, DMSO-d₆) δ 9.92 (s, 1H), 8.07 (s, 1H), 7.69 (dd, J=7.9, 1.7 Hz, 1H), 7.38-7.27 (m, 2H), 7.03-6.96 (m, 7H), 6.93-6.88 (m, 1H), 4.35 (s, 1H), 3.73 (s, 1H), 2.78 (s, 2H), 2.54 (d, J=7.1 Hz, 2H), 1.87 (s, 1H), 1.55 (s, 2H), 1.11 (qd, J=12.1, 4.1 Hz, 2H), ¹⁹F NMR (377 MHz, DMSO-d₆) δ −113.89 (s), SM (IS): 430.1 m/z: [M+1].

Scheme 11 Synthesis of compounds 47-49.

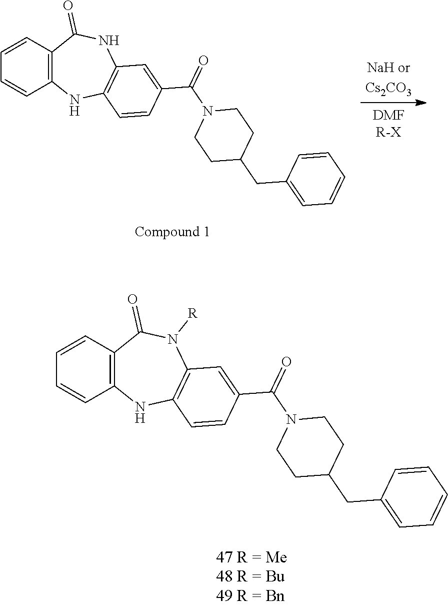

### 8-(4-benzylpiperidine-1-carbonyl)-10-methyl-5,10-dihydro-11H dibenzo[b,e][1,4]diazepin-11-one (47)

Compound 1 (100 mg, 0.243 mmol) was dissolved in 1 ml of DMF, then Cs₂CO₃ (158 mg, 0.486 mg) was added at room temperature following by Mel (16 ml, 0.267 mmol). The reaction mixture was stirred overnight at room temperature and then diluted with H₂O (20 ml). The obtained solid was filtered, washed with 5 ml of H₂O and then purified by column chromatography on silica gel (DCM/Methanol (99:1)) to afford compound 47 as a white solid (80 mg, 78%). ¹H NMR (400 MHz, DMSO-d₆) δ 8.13 (s, 1H), 7.67 (dd, J=7.9, 1.7 Hz, 1H), 7.40-7.33 (m, 1H), 7.32-7.25 (m, 3H), 7.20-7.12 (m, 4H), 7.12-7.06 (m, 2H), 7.00-6.95 (m, 1H), 4.50-4.27 (m, 1H), 3.80-3.56 (m, 1H), 3.61 (s, 3H), 3.10-2.82 (m, 1H), 2.72-2.70 (m, 1H), 2.53 (s, 2H), 1.80-1.74 (m, 1H), 1.67-1.47 (m, 2H), 1.25-1.02 (m, 2H); ¹³C NMR (101 MHz, DMSO) δ 168.1, 167.5, 151.2, 144.8, 140.0, 134.6, 132.6, 132.3, 131.5, 129.0, 128.2, 125.8, 124.3, 123.8, 122.0, 121.5, 119.9, 118.8, 42.1, 40.15, 37.7, 37.5, 31.5. SM (IS): 426.2 m/z: [M+1]; HRMS (ESI) [M+H]⁺ calcd for C₂₇H₂₈N₃O₂: 426.2103, found: 426.2176.

### 8-(4-benzylpiperidine-1-carbonyl)-10-butyl-5,10-dihydro-11H-dibenzo[b,e][1,4]diazepin-11-one (48)

Compound 1 (40 mg, 0.097 mmol) was dissolved in 0.5 ml of DMF, then NaH (5 mg, 0.194 mmol) was added at room temperature following by BuI (12 ml, 0.106 mmol). The reaction mixture was stirred overnight at room temperature. The mixture was diluted with H₂O (20 ml). The obtained solid was filtered, washed with 5 ml of H₂O and purified by column chromatography on silica gel (DCM/Methanol (99:1)) to afford compound 48 as a white solid (22.5 mg, 50%); ¹H NMR (400 MHz, DMSO-d₆) δ 8.09 (s, 1H), 7.68 (dd, J=7.8, 1.7 Hz, 1H), 7.43-7.37 (m, 2H), 7.37-7.31 (m, 2H), 7.24 (ddd, J=8.3, 5.9, 1.9 Hz, 4H), 7.14 (ddd, J=16.2, 8.2, 1.5 Hz, 2H), 7.03 (ddd, J=8.1, 7.3, 1.2 Hz, 1H), 4.57-4.36 (m, 1H), 4.08 (s, 2H), 3.80-3.56 (m, 1H) 3.10-2.86 (m, 2H), 2.59 (s, 2H), 1.88-1.77 (m, 1H), 1.72-1.55 (m, 2H), 1.50 (dt, J=14.2, 6.8 Hz, 2H), 1.32 (dq, J=14.2, 7.3 Hz, 2H), 1.26-1.15 (m, 2H), 0.86 (t, J=7.3 Hz, 3H). SM (IS): 468.1 m/z: [M+1].

### 10-benzyl-8-(4-benzylpiperidine-1-carbonyl)-5,10-dihydro-11H-dibenzo[b,e][1,4]diazepin-11-one (49)

Compound 1 (40 mg, 0.097 mmol) was dissolved in 0.5 ml of DMF, then Cs₂CO₃ (63 mg, 0.191 mmol) was added at room temperature followed by benzylbromide (13 ml, 0.106 mmol). The reaction mixture was stirred overnight at room temperature and then diluted with H₂O (20 ml). The obtained solid was filtered, washed with 5 ml of H₂O and purified by column chromatography on silica gel (DCM/Methanol (99:1)) to afford compound 49 as a white solid (38 mg, 80%); ¹H NMR (400 MHz, DMSO-d₆) δ 8.13 (s, 1H), 7.69 (dd, J=7.8, 1.5 Hz, 1H), 7.39 (ddd, J=8.5, 7.3, 1.6 Hz, 1H), 7.35-7.24 (m, 7H), 7.22-7.13 (m, 4H), 7.16-7.07 (m, 2H), 7.05-6.96 (m, 2H), 5.27 (s, 2H), 4.54-4.06 (m, 1H), 2.94-2.50 (m, 1H), 2.48 (d, J=6.7 Hz, 1H), 1.77-1.65 (m, 1H), 1.61-1.38 (m, 2H), 1.27-1.23 (m, 1H), 1.04-0.97 (m, 1H), 0.90-0.84 (m, 1H). SM (IS): 502.6 m/z: [M+1].

### Synthesis of Compounds 51 and 52.

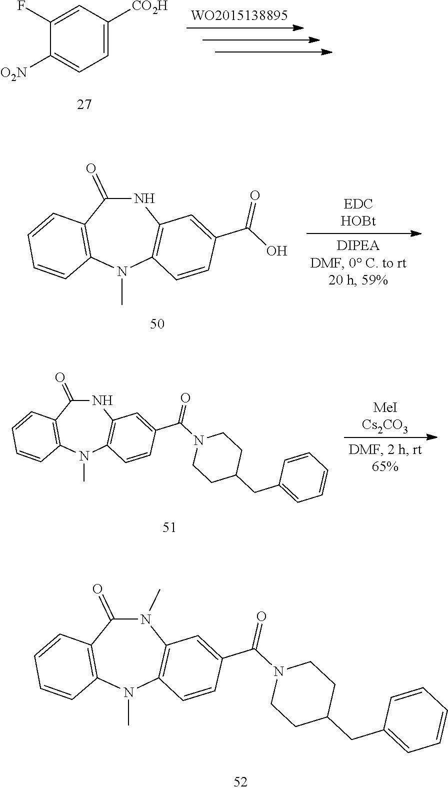 [SMILES: O=C(O)c1ccc([N+](=O)[O-])c(F)c1 ; CN1c2ccc(C(=O)O)cc2NC(=O)c2ccccc21 ; CN1c2ccc(C(=O)N3CCC(Cc4ccccc4)CC3)cc2NC(=O)c2ccccc21 ; CN1C(=O)c2ccccc2N(C)c2ccc(C(=O)N3CCC(Cc4ccccc4)CC3)cc21]

### 8-(4-benzylpiperidine-1-carbonyl)-5-methyl-5,10-dihydro-11H-dibenzo[b,e][1,4]diazepin-11-one (51)

Compound 51 was prepared from compound 50 (120 mg, 0.447 mmol, WO2015138895) and 4-benzylpiperidine (104 ml, 0.581 mmol) following general procedure I. Column chromatography: DCM/Methanol (99:1); white solid (113 mg, 59%); ¹H NMR (400 MHz, DMSO-d₆) δ 10.30 (s, 1H), 7.64 (dd, J=7.7, 2.0 Hz, 1H), 7.50 (ddd, J=8.2, 7.3, 1.8 Hz, 1H), 7.27 (dd, J=8.3, 6.5 Hz, 2H), 7.24-7.14 (m, 5H), 7.13-7.07 (m, 2H), 7.04 (d, J=2.0 Hz, 1H), 4.45-4.40 (m, 1H), 3.66-3.6 (m, 1H), 3.28 (s, 3H), 2.99-2.82 (m, 1H), 2.76-2.63 (m, 2H), 2.52 (s, 1H), 1.82-1.71 (m, 1H), 1.69-1.45 (m, 2H), 1.20-1.03 (m, 2H); ¹³C NMR (101 MHz, DMSO-d₆) δ: 168.2, 168.0, 152.4, 145.0, 140.0, 132.9, 132.1, 131.7, 131.0, 129.0, 128.1, 126.7, 125.8, 123.3, 122.8, 119.8, 119.0, 117.7, 42.1, 40.15, 39.94, 37.8, 37.5, 31.9; SM (IS): 426.1 m/z: [M+1].

### 8-(4-benzylpiperidine-1-carbonyl)-5,10-dimethyl-5,10-dihydro-11H-dibenzo[b,e][1,4]diazepin-11-one (52)

Compound 51 (50 mg, 0.117 mmol) was dissolved in 1 ml of DMF, then Cs₂CO₃ (76 mg, 0.235 mg) was added at room temperature followed by Mel (14 ml, 0.235 mmol). The reaction mixture was stirred at room temperature for 2 h and then diluted with H₂O (20 ml). The obtained solid was filtered, washed with 5 ml of H₂O and purified by column chromatography on silica gel (DCM/Methanol (99:1)) to afford compound 52 as a white solid (33 mg, 65%); ¹H NMR (400 MHz, DMSO-d₆) δ 7.62 (dd, J=7.7, 1.7 Hz, 1H), 7.46 (ddd, J=8.7, 7.7, 1.9 Hz, 1H), 7.33 (d, J=1.9 Hz, 1H), 7.31-7.24 (m, 3H), 7.20-7.14 (m, 5H), 7.09 (td, J=7.5, 1.0 Hz, 1H), 4.53-4.23 (m, 1H), 3.60-3.57 (m, 1H), 3.43 (s, 3H), 3.35 (s, 3H), 2.99-2.93 (m, 1H), 2.72-2.63 (m, 1H), 2.52 (s, 2H), 1.85-1.69 (m, 1H), 1.68-1.43 (m, 2H), 1.20-1.05 (m, 2H); ¹³C NMR (101 MHz, DMSO) δ 167.8, 167.5, 153.0, 147.9, 140.0, 136.5, 132.4, 131.5, 129.0, 128.1, 126.6, 125.8, 124.2, 122.8, 121.7, 118.7, 116.6, 42.0, 37.4, 37.4, 37.0, 31.9; SM (IS): m/z: 440.5 [M+1].

### Synthesis of Compound 58.

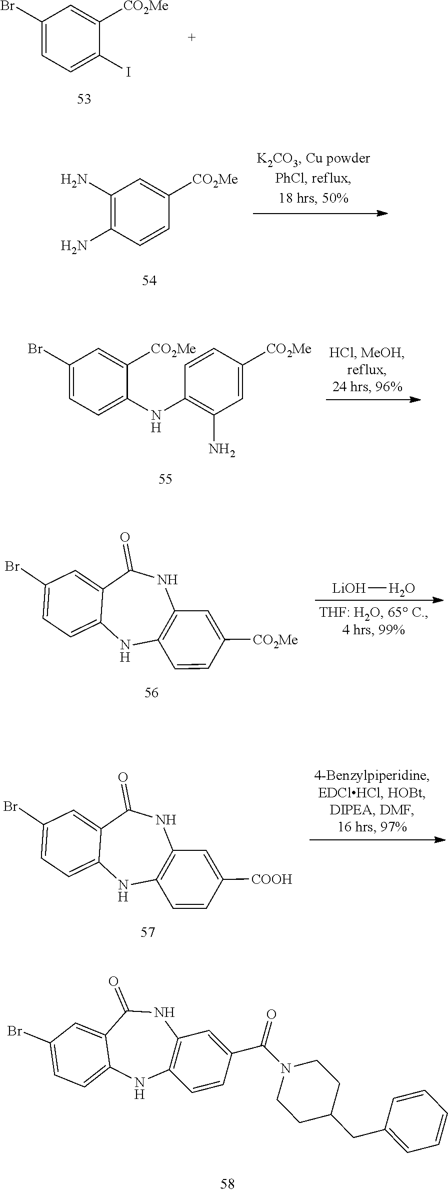

### Methyl 2-((2-amino-4-(methoxycarbonyl)phenyl)amino)-5-bromobenzoate (55)

To a solution of methyl-3,4-diaminobenzoate 54 (2.051 g, 6.01 mmol) in chlorobenzene (20 mL) was added methyl 5-bromo-2-iodobenzoate 53 (1 g, 6.01 mmol), K₂CO₃ (0.87 g, 6.30 mmol), and Cu (0.382 g, 6.01 mmol). The resulting mixture was heated at reflux for 18 hours. While hot, the mixture was filtered through a thin layer of diatomaceous earth and the cake was washed with dichloromethane. The filtrate was concentrated and the crude product purified by flash chromatography on silica gel, eluting with 10%-100% CH₂Cl₂/hexanes gradient to yield title compound 55 (1.1 g, 50%). ¹H NMR (400 MHz, Chloroform-d) δ 9.14 (bs, 1H), 8.09 (d, J=2.4 Hz, 1H), 7.49 (s, 1H), 7.43 (d, J=8.2 Hz, 1H), 7.38 (dd, J=9.0, 2.5 Hz, 1H), 7.21 (d, J=8.2 Hz, 1H), 6.72 (d, J=9.0 Hz, 1H), 3.93 (s, 3H), 3.90 (s, 3H). LR-MS calculated for C₁₆H₁₅BrN₂O₄ 378.02, found 379.3, 381.3.

### Methyl 2-bromo-11-oxo-10,11-dihydro-5H-dibenzo[b,e][1,4]diazepine-8-carboxylate (56)

To compound 55 (0.72 g, 1.9 mmol) in methanol (35 mL) was added concentrated HCl (7 mL) and the mixture was heated to reflux overnight. After cooling to room temperature, the reaction mixture was filtered and the cake was washed with water to yield title compound 56 (0.63 g, 96%). ¹H NMR (400 MHz, DMSO-d₆) δ 10.09 (s, 1H), 8.52 (s, 1H), 7.78 (d, J=2.5 Hz, 1H), 7.59-7.52 (m, 3H), 7.05 (d, J=8.2 Hz, 1H), 6.96 (d, J=8.6 Hz, 1H), 3.80 (s, 3H). LR-MS calculated for C₁₅H₁₁BrN₂O₃ 345.99, found 347.0, 349.0.

### 2-Bromo-11-oxo-10,11-dihydro-5H-dibenzo[b,e][1,4]diazepine-8-carboxylic acid (57)

To a stirred solution of compound 4 (0.6 g, 1.7 mmol) in THF:H₂O (7:3, 45 mL) was added lithium hydroxide monohydrate (0.435 g, 10.4 mmol) at room temperature. The resulting solution was stirred at 65° C. for 4 h. The reaction was monitored by TLC and after completion of reaction, the volatiles were removed in vacuo. The pH of the residue was acidified to a pH of ˜4 with 2N HCl. The precipitated solid was filtered, washed with water (20 mL) and dried in vacuo to afford compound 5 (0.570 g, 99%). ¹H NMR (400 MHz, DMSO-d₆) δ 12.65 (s, 1H), 10.00 (s, 1H), 8.62 (s, 1H), 7.70 (d, J=2.5 Hz, 1H), 7.50-7.43 (m, 3H), 7.04 (d, J=8.3 Hz, 1H), 6.99 (d, J=8.7 Hz, 1H). LR-MS calculated for C₁₉H₉BrN₂O₃ 331.97, found 333.3, 335.3.

### 8-(4-Benzylpiperidine-1-carbonyl)-2-bromo-5,10-dihydro-11H-dibenzo[b,e][1,4]diazepin-11-one (58)

To a solution of compound 57 (0.3 g, 0.90 mmol) in 5 ml DMF was added N-(3-dimethylaminopropyl)-N′-ethylcarbodiimide hydrochloride (EDCI) (0.276 g, 1.44 mmol), N-hydroxybenzotriazole (HOBt) (0.194 g, 1.44 mmol), 4-benzylpiperidine (0.205 mL, 1.17 mmol) followed by DIPEA (0.470 mL, 2.70 mmol). The reaction mixture was stirred at room temperature for 16 hours, quenched with water and then extracted with ethyl acetate. The combined organic layers were washed with brine, dried over sodium sulfate and concentrated under vacuum. The residue was then suspended in 3 mL of ethyl acetate before addition of 30 mL of hexanes. The precipitate was filtered and washed with hexane (10 mL) to afford title compound 58 (0.430 g, 97%). ¹H NMR (400 MHz, Methanol-d₄) δ 7.85 (d, J=2.4 Hz, 1H), 7.44 (dd, J=8.6, 2.5 Hz, 1H), 7.28-7.23 (m, 2H), 7.18-7.14 (m, 3H), 7.06-6.97 (m, 3H), 6.85 (d, J=8.6 Hz, 1H), 4.63-4.44 (m, 1H), 3.88-3.63 (m, 1H), 3.11-2.92 (m, 1H), 2.91-2.08 (m, 1H), 2.86-2.69 (m, 1H), 2.56 (d, J=5.7 Hz, 2H), 1.91-1.54 (m, 3H), 1.29-1.11 (m, 2H). LR-MS calculated for C₂₆H₂₄BrN₃O₂ 489.10, found 490.0, 491.9.

### Synthesis of Compound 59

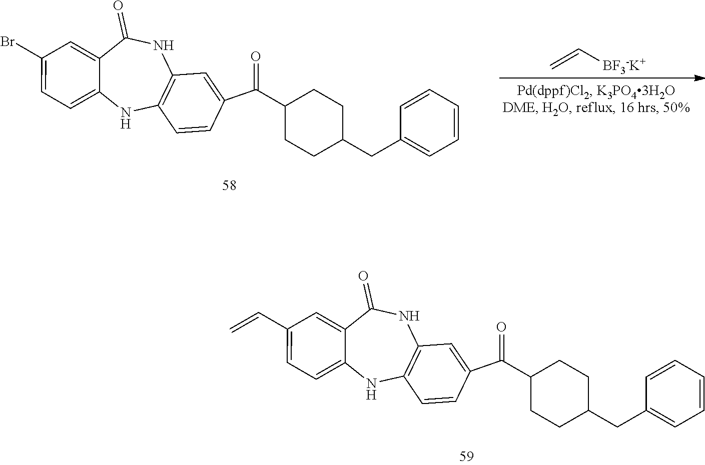

### 8-(4-benzylpiperidine-1-carbonyl)-2-vinyl-5,10-dihydro-11H-dibenzo[b,e][1,4]diazepin-11-one (59)

A mixture of compound 58 (0.1 g, 0.204 mmol) and potassium vinyltrifluoroborate (36 mg, 0.265 mmol), [1,1′-bis(diphenylphosphino)ferrocene]dichloropalladium (II) (0.030, 0.04 mmol) and K₃PO₄·3H₂O (0.143 g, 0.674 mmol) in DME:H₂O (2:1, 4 mL) was heated to reflux for 16 hours. After cooling the reaction mixture was partitioned between ethyl acetate and water. The ethyl acetate layer was washed with brine, dried over magnesium sulfate, filtered and concentrated. The residue was purified by flash column chromatography on silica gel using CH₂Cl₂:MeOH to provide title compound 59 (50 mg, 50%). ¹H NMR (400 MHz, Methanol-d₄) δ 7.82 (d, J=2.2 Hz, 1H), 7.47 (dd, J=8.4, 2.2 Hz, 1H), 7.29-7.24 (m, 2H), 7.19-7.15 (m, 3H), 7.06-6.98 (m, 3H), 6.91 (d, J=8.4 Hz, 1H), 6.65 (dd, J=17.6, 11.0 Hz, 1H), 5.68 (dd, J=17.6, 0.9 Hz, 1H), 5.16 (dd, J=10.9, 0.9 Hz, 1H), 4.62-4.45 (m, 1H), 3.85-3.70 (m, 1H), 3.12-2.94 (m, 1H), 2.86-2.69 (m, 1H), 2.58 (d, J=7.1 Hz, 2H), 1.88-1.55 (m, 3H), 1.29-1.11 (m, 2H). LR-MS calculated for C₂₈H₂₇N₃O₂ 437.21, found 438.5.

### Synthesis of Compounds 60-66.

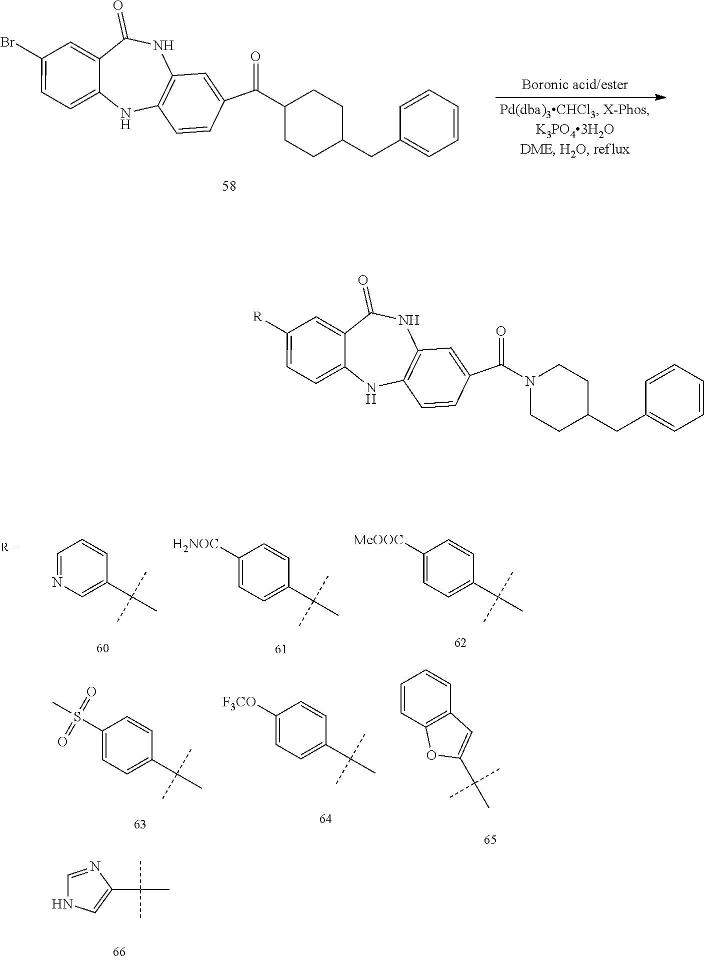

### 8-(4-benzylpiperidine-1-carbonyl)-2-(pyridin-3-yl)-5,10-dihydro-11H-dibenzo[b,e][1,4]diazepin-11-one (60)

A mixture of compound 58 (0.025 g, 0.051 mmol) and 3-pyridineboronic acid 1,3-propanediol ester (0.013, 0.076 mmol), Pd₂(dba)₃ CHCl₃ (5.2 mg, 0.005 mmol), X-Phos (14.6 mg, 0.030 mmol) and K₃PO₄ 3H₂O (0.035 g, 0.168 mmol) in DME:H₂O (2:1, 4 mL) was heated to reflux for 48 hours. After cooling the reaction to room temperature, the mixture was partitioned between ethyl acetate and water. The ethyl acetate layer was washed with brine, dried on magnesium sulfate, filtered and concentrated. The residue was purified by flash column chromatography on silica gel using CH₂Cl₂:MeOH to provide title compound 59 (19 mg, 78%). ¹H NMR (400 MHz, DMSO-d₆) δ 10.04 (s, 1H), 8.83 (d, J=2.6 Hz, 1H), 8.53 (dd, J=4.8, 1.6 Hz, 1H), 8.32 (s, 1H), 8.03-7.99 (m, 2H), 7.76 (dd, J=8.4, 2.4 Hz, 1H), 7.48-7.44 (m, 1H), 7.30-7.27 (m, 2H), 7.20-7.12 (m, 4H), 7.05-6.99 (m, 3H), 4.50-4.18 (m, 1H), 3.85-3.50 (m, 1H), 3.05-2.58 (m, 2H), 2.53 (d, J=7.7 Hz, 2H), 1.84-1.71 (m, 1H), 1.70-1.41 (m, 2H), 1.18-1.06 (m, 2H). LR-MS calculated for C₃₁H₂₈N₄O₂ 488.22, found 489.2.

### 4-(8-(4-benzylpiperidine-1-carbonyl)-11-oxo-10,11-dihydro-5H-dibenzo[b,e][1,4]diazepin-2-yl)benzamide (61)

Compound 61 was synthetized by following the chemistry used to prepare compound 60 by substituting 4-aminocarbonylphenylboronic acid for 3-pyridineboronic acid 1,3-propanediol ester (63% yield). ¹H NMR (400 MHz, DMSO-d₆) δ 10.03 (s, 1H), 8.31 (s, 1H), 8.09-7.91 (m, 4H), 7.77 (dd, J=8.5, 2.4 Hz, 1H), 7.69 (d, J=8.5 Hz, 2H), 7.38 (s, 1H), 7.32-7.25 (m, 2H), 7.22-7.09 (m, 4H), 7.07-6.97 (m, 3H), 4.58-4.14 (m, 1H), 3.85-3.54 (m, 1H), 3.05-2.53 (m, 4H), 1.87-1.69 (m, 1H), 1.68-1.43 (s, 2H), 1.19-1.06 (m, 2H). LR-MS calculated for C₃₃H₃₀N₄O₃ 530.23, found 531.2.

### Methyl 4-(8-(4-benzylpiperidine-1-carbonyl)-11-oxo-10,11-dihydro-5H-dibenzo[b,e][1,4]diazepin-2-yl)benzoate (62)

Compound 62 was synthetized by following the chemistry used to prepare compound 60 by substituting for 4-methoxycarbonylphenylboronic acid for 3-pyridineboronic acid 1,3-propanediol ester (23% yield). ¹H NMR (400 MHz, DMSO-d₆) δ 10.04 (s, 1H), 8.36 (s, 1H), 8.15-7.95 (m, 3H), 7.88-7.71 (m, 3H), 7.41-7.22 (m, 2H), 7.23-7.09 (m, 4H), 7.07-6.97 (m, 3H), 4.54-4.17 (m, 1H), 3.87 (s, 3H), 3.79-3.52 (m, 1H), 3.08-2.54 (m, 4H), 1.84-1.71 (m, 1H), 1.67-1.45 (m, 2H), 1.18-1.03 (m, 2H). LR-MS calculated for C₃₄H₃₁N₃O₄ 545.23, found 546.0.

### 8-(4-benzylpiperidine-1-carbonyl)-2-(4-(methylsulfonyl)phenyl)-5,10-dihydro-11H-dibenzo[b,e][1,4]diazepin-11-one (63)

Compound 63 was synthetized by following the chemistry used to prepare compound 60 by substituting 4-(methanesulfonyl)phenylboronic acid for 3-pyridineboronic acid 1,3-propanediol ester (85% yield). ¹H NMR (400 MHz, DMSO-d₆) δ 10.05 (d, J=2.1 Hz, 1H), 8.38 (s, 1H), 8.07 (d, J=2.4 Hz, 1H), 7.99-7.94 (m, 2H), 7.92-7.86 (m, 2H), 7.79 (dd, J=8.5, 2.4 Hz, 1H), 7.33-7.24 (m, 2H), 7.21-7.11 (m, 4H), 7.06-6.97 (m, 3H), 4.55-4.16 (m, 1H), 3.75-3.53 (m, 1H), 3.24 (s, 3H), 3.05-2.53 (m, 4H), 1.83-1.68 (m, 1H), 1.67-1.45 (m, 2H), 1.18-1.03 (m, 2H). LR-MS calculated for C₃₃H₃₁N₃O₄S 565.20, found 566.1.

### 8-(4-benzylpiperidine-1-carbonyl)-2-(4-(trifluoromethoxy)phenyl)-5,10-dihydro-11H-dibenzo[b,e][1,4]diazepin-11-one (64)

Compound 12 was synthetized by following the chemistry used to prepare compound 60 by substituting 4-(trifluoromethoxy)phenylboronic acid for 3-pyridineboronic acid 1,3-propanediol ester (63% yield). ¹H NMR (400 MHz, DMSO-d₆) δ 10.04 (s, 1H), 8.30 (s, 1H), 7.98 (d, J=2.4 Hz, 1H), 7.75-7.64 (m, 3H), 7.49-7.35 (m, 2H), 7.27 (dd, J=8.3, 6.4 Hz, 2H), 7.21-7.08 (m, 4H), 7.08-6.93 (m, 3H), 4.58-4.17 (m, 1H), 3.83-3.48 (m, 1H), 3.07-2.53 (m, 4H), 1.83-1.68 (m, 1H), 1.67-1.40 (m, 2H), 1.17-1.05 (m, 2H). LR-MS calculated for C₃₃H₂₈F₃N₃O₃ 571.20, found 572.2.

### 2-(benzofuran-2-yl)-8-(4-benzylpiperidine-1-carbonyl)-5,10-dihydro-11H-dibenzo[b,e][1,4]diazepin-11-one (65)

Compound 65 was synthetized by following the chemistry used to prepare compound 60 by substituting 2-benzofuranylboronic acid for 3-pyridineboronic acid 1,3-propanediol ester (52% yield). ¹H NMR (400 MHz, DMSO-d₆) δ 10.07 (s, 1H), 8.44 (s, 1H), 8.25 (d, J=2.2 Hz, 1H), 7.91 (dd, J=8.4, 2.3 Hz, 1H), 7.65-7.56 (m, 2H), 7.38-7.22 (m, 5H), 7.22-7.09 (m, 4H), 7.07-6.94 (m, 3H), 4.61-4.11 (m, 1H), 3.87-3.51 (m, 1H), 3.05-2.53 (m, 4H), 1.84-1.69 (m, 1H), 1.68-1.44 (m, 2H), 1.26-1.00 (m, 2H). LR-MS calculated for C₃₄H₂₉N₃O₃ 527.22, found 528.2.

### 8-(4-benzylpiperidine-1-carbonyl)-2-(1H-pyrazol-4-yl)-5,10-dihydro-11H-dibenzo[b,e][1,4]diazepin-11-one (66)

Compound 66 was synthetized by following the chemistry used to prepare compound 60 by substituting 1H-pyrazole-4-boronic acid for 3-pyridineboronic acid 1,3-propanediol ester (62% yield). ¹H NMR (400 MHz, DMSO-d₆) δ 12.90 (s, 1H), 9.98 (s, 1H), 8.06 (s, 2H), 7.87 (d, J=2.3 Hz, 2H), 7.61 (dd, J=8.3, 2.3 Hz, 1H), 7.31-7.25 (m, 2H), 7.21-7.15 (m, 3H), 7.05-6.96 (m, 4H), 4.68-4.17 (m, 1H), 3.89-3.51 (m, 1H), 3.03-2.53 (m, 4H), 1.87-1.70 (m, 1H), 1.67-1.37 (m, 2H), 1.20-1.01 (m, 2H). LR-MS calculated for C₂₉H₂₇N₅O₂ 477.21, found 478.2.

### Synthesis of Compound 67.

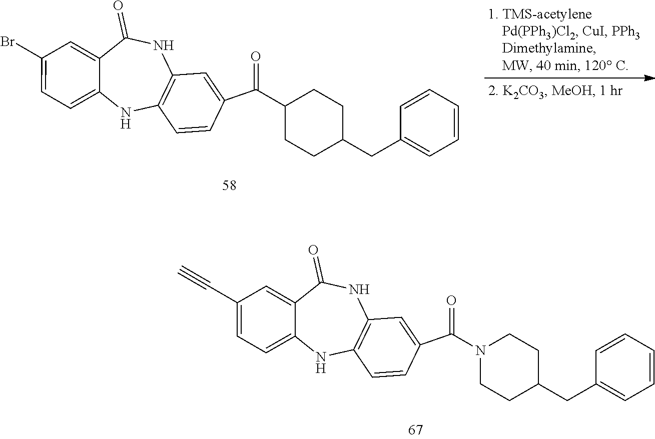 [SMILES: O=C1Nc2cc(C(=O)C3CCC(Cc4ccccc4)CC3)ccc2Nc2ccc(Br)cc21 ; C#Cc1ccc2c(c1)C(=O)Nc1cc(C(=O)N3CCC(Cc4ccccc4)CC3)ccc1N2]

### 8-(4-benzylpiperidine-1-carbonyl)-2-ethynyl-5,10-dihydro-11H-dibenzo[b,e][1,4]diazepin-11-one (67)

A mixture of compound 58 (0.100 g, 0.204 mmol), Pd(PPh₃)Cl₂ (7.1 mg, 0.01 mmol), CuI (1.9 mg, 0.01 mmol), triphenylphosphine (10.7 mg, 0.04 mmol), trimethylsilylacetylene (31 QL, 0.224 mmol), and diethylamine (0.29 mL, 2.76 mmol) in dimethylformamide (1 mL) was heated at 120° C. for 40 min under microwave irradiation. The reaction mixture was filtered and washed with dichloromethane. The filtrate was concentrated under reduced pressure and the residue was purified by flash chromatography on silica gel to obtain 63 mg of trimethylsilyl protected intermediate. This intermediate was then treated with potassium carbonate (68 mg, 4 mmol) in methanol (3 mL) and the reaction mixture stirred at room temperature for 1 h. The reaction mixture was filtered, and the filtrate was concentrated under reduced pressure to give a residue, which was purified by flash chromatography on silica gel to give title compound 67 (46 mg, 53% over two steps). ¹H NMR (400 MHz, DMSO-d₆) δ 9.99 (s, 1H), 8.39 (s, 1H), 7.75 (d, J=2.1 Hz, 1H), 7.42 (dd, J=8.3, 2.2 Hz, 1H), 7.33-7.23 (m, 2H), 7.21-7.09 (m, 3H), 7.04-6.65 (m, 4H), 4.50-4.17 (m, 1H), 4.07 (s, 1H), 3.83-3.48 (m, 1H), 3.08-2.53 (m, 4H), 1.87-1.68 (m, 1H), 1.67-1.43 (m, 2H), 1.19-1.01 (m, 2H). LR-MS calculated for C₂₈H₂₅N₃O₂ 435.19, found 436.3.

### Example 2

### Cellular Toxicity Assays

The toxicity of the compounds was assessed in human PBM, CEM (human lymphoblastoid), and Huh-7 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide was included as positive cytotoxic control, and untreated cells exposed to solvent were included as negative controls. The cytotoxicity IC₅₀ was obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11). The results are shown in Table 1 below:

TABLE 1

| | Cytotoxicity (IC₅₀, μM) | | |
|---|---|---|---|
| Structure | PBM | CEM | Huh-7 |
| 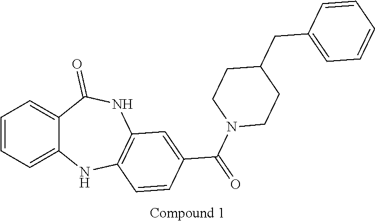 [SMILES: O=C1Nc2cc(C(=O)N3CCC(Cc4ccccc4)CC3)ccc2Nc2ccccc21] | 92.4 | 11.0 | 10.4 |

### Example 3

### RORα Activity by Luciferase Reporter

Huh-7 cells were transfected with a luciferase reporter plasmid containing the miR-122 promoter (extends to −900 from the transcription start site) with intact wild-type (WT) RORα response element (RORE) or mutated RORE (mut). The cells were treated with Compound 1 one day (24 hours) post-transfection at the indicated concentrations. Luciferase expression was measured after 24 hours of treatment and normalized to Renilla Luciferase activity expressed from a co-transfected pRL plasmid. The pRL Vector, which provides constitutive expression of Renilla luciferase, was used in combination with a firefly luciferase vector to co-transfect cells. Expression of Renilla luciferase provides an internal control value to which expression of the experimental firefly luciferase reporter gene may be normalized.

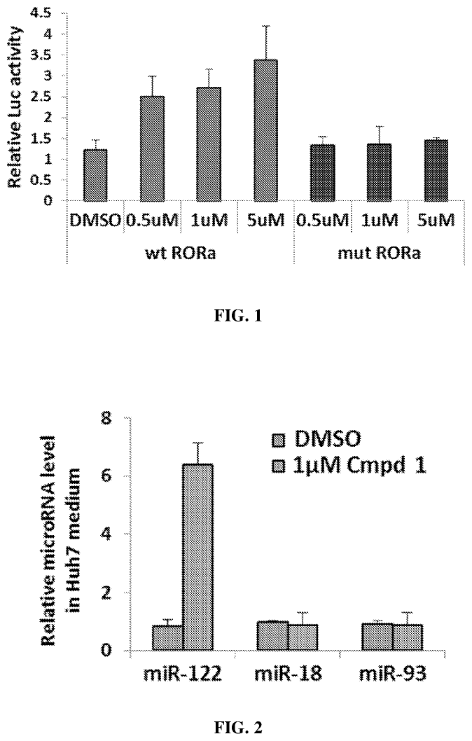

The results show a dose-dependent increase of luciferase expression for Compound 1 with the use of WT RORE. Mutating the RORE negates activity of Compound 1. These results, shown in FIG. 1, indicate RORα activity of Compound 1 as an agonist.

This assay can be used to evaluate other compounds described herein. Where compounds increase luciferase expression, they are RORα agonists, and where they decrease luciferase expression, they are RORα antagonists (or partial agonists or allosteric inhibitors).

### Example 4

Expression of RORα-Regulated microRNA.

Huh-7 cells were treated with 1 μM Compound 1 or vehicle (DMSO) for 24 hours. Secreted microRNA levels in the medium of Huh-7 cells were analyzed by qRT-PCR and were normalized to spiked C. elegans miR-39. miR-18 and miR-93 served as controls for secreted microRNA and were not affected following Compound 1 addition. The results are shown in FIG. 2.

### Example 5

### Modulation of Th17 Populations

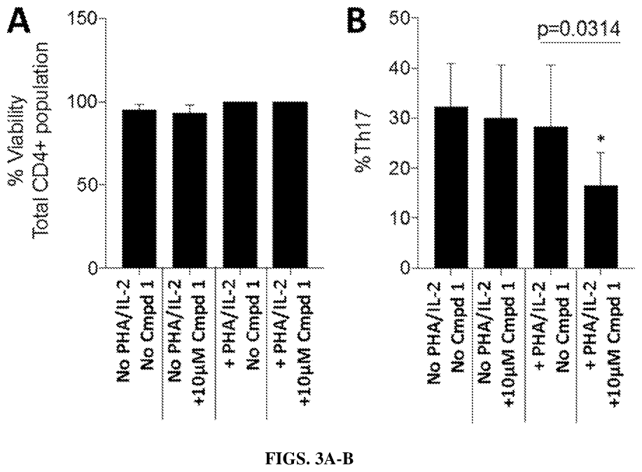

Human peripheral blood mononuclear cells (PBMCs) were isolated from four healthy donors. Four experiments were conducted and analyzed by flow cytometry over 3 day assay. The control group had no drug treatment, a second were treated with 10 μM Compound 1, a third group was stimulated with PHA/IL-2 without treatment, and the fourth group was stimulated with PHA/IL-2 and incubated with 10 μM Compound 1. These results, shown in FIG. 3A, show that Compound 1 has no effect on total viability of CD4⁺ T cells even under PHA/IL-2 stimulation. Further, Compound 1 has no effect on Th17 populations in the absence of PHA/IL-2 stimulation. As shown in FIG. 3B, Compound 1 decreases Th17 total population in PBM cells relative to vehicle-control in the presence of PHA/IL-2 stimulation.

### Example 6

### Modulation of RORα-Regulated Genes in C57BL/6 Mice

Healthy C57BL/6 mice were injected i.p. once with 7.5 mg/kg Compound 1 or saline control. Mice were sacrificed at 1, 2, and 7 day time points post-injection. miR-122 and Gpase 6 mRNA levels were determined by qRT-PCR for each time point. MicroRNA levels were normalized to RNU6; plasma miR-122 was normalized to spiked in C. elegans miR-39; and mRNA levels were normalized to HPRT.

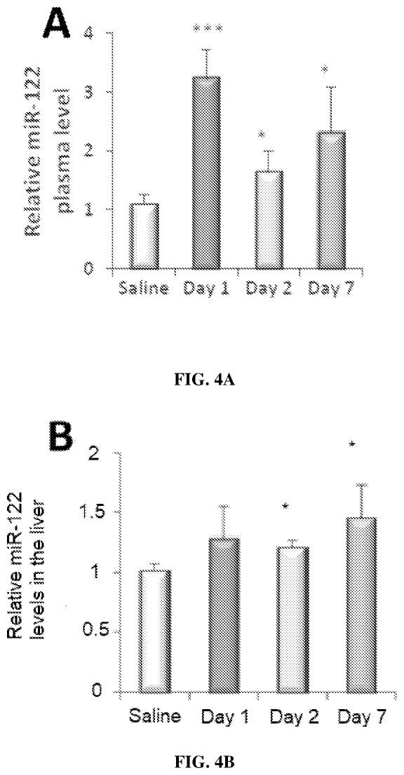

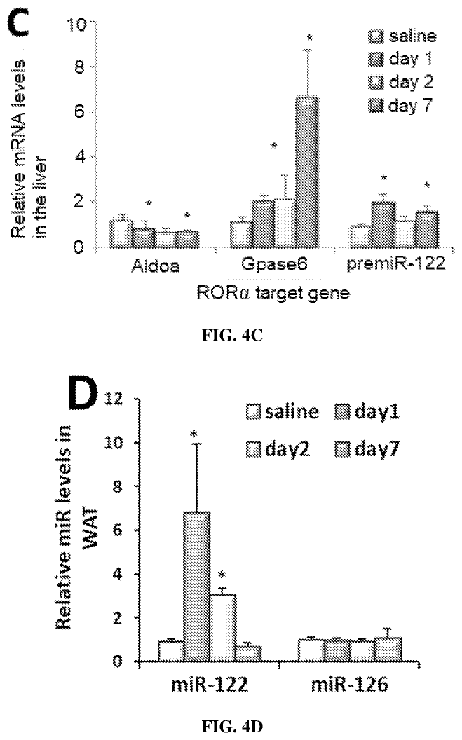

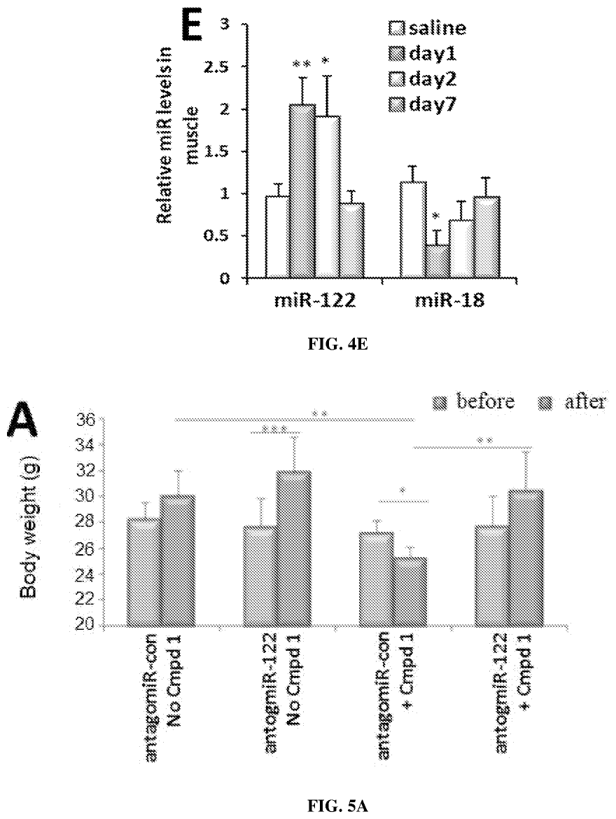

These results, shown in FIGS. 4A-E, show that after administration of Compound 1, miR-122 levels are increased in plasma and liver up to 7 days post-injection. Further, the RORα-regulated gene Gpase6 is significantly up-regulated up to 7 days post-injection of Compound 1.

### Example 7

### Enhanced Secretion of Mir-122 in C57BL/6 Mice Due to RORα Modulation

C57BL/6 mice were fed a 50% high fat diet (HFD) for four weeks. The control cohort received three hydrodynamic tail vein injections of a 5 μg antagomiR-control and six i.p injections of saline over three weeks. A second cohort was hydrodynamic tail vein injected with 5 μg antagomiR-122 (the reverse complement that inhibits activity of miR-122) three times and i.p injections of saline six times over three weeks. A third cohort was injected i.p. with Compound 1 (7.5 mg/kg) twice a week plus antagomiR-control once a week over the course of 3 weeks. The final cohort was injected i.p. with Compound 1 (7.5 mg/kg) twice a week plus antagomiR-122 injections once a week over the course of 3 weeks.

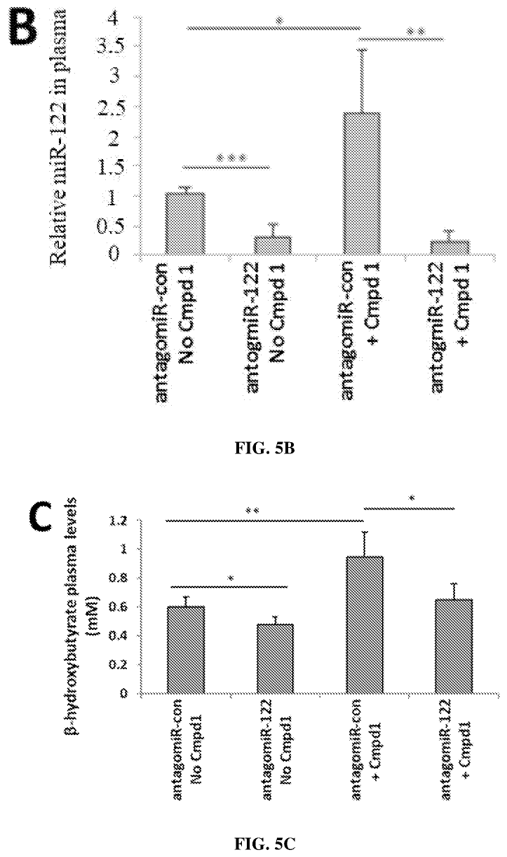

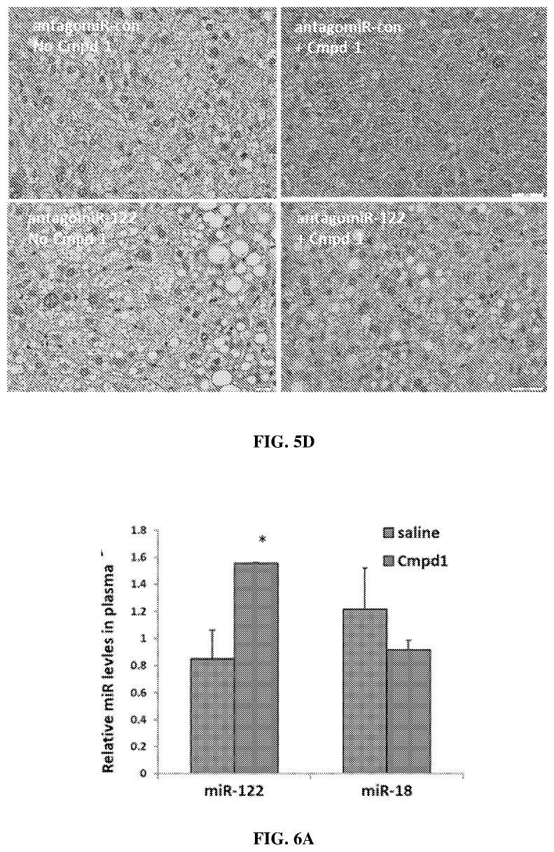

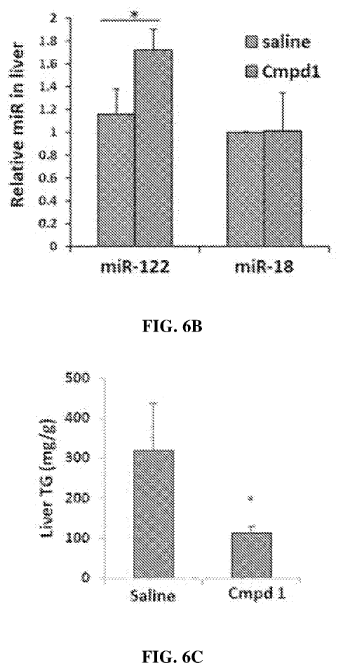

As shown in FIGS. 5A-D, secretion of miR-122 was enhanced when treated with Compound 1, which could be reduced to baseline levels with co-administration of antagomir-122.

### Detailed Methods

Cell Culture. HCC-derived human cell lines: Huh7 were cultured in DMEM supplemented with 10% fetal calf serum (FCS), 1% penicillin/streptomycin.

Plasmids. The human miR-122 promoter fragments spanning the region from −900 bp relative to the transcription start site (TSS) (plasmids PmiR-122-900) were generated as described previously (1). Mutating the RORα site in the promoter region was performed by PCR using primers P1 and P2, as described previously (2). All primers used to generate the plasmids are described Table 2.

Luciferase assay. For Luciferase assays, cells grown in 24 well plates were co-transfected with a luciferase reporter plasmid (50 ng) and 1 ng of Renilla Luciferase vector (PRL, Promega) using the TransIT-LT 1 (Mirus) transfection reagent (MIR 2300, Madison, WI). Firefly and Renilla luciferase activity was assessed using the Dual Luciferase Reporter Assay system (Promega). Readings were taken in triplicates on a Mithras LB 940 Luminometer (Berthold Technologies).

RNA extraction and quantitative Real-Time PCR analysis. Total RNA, including small RNAs, were isolated from 200 L of plasma or culture media samples using the miRNeasy Mini kit (Qiagen, Valencia, CA, USA) with 2 minor modifications. First, 200 μl of plasma or culture media were lysed with 1 ml of Qiazol solution. Second, a 50 pmol/1 of synthesized single strand Caenorhabditis elegans miRNA (cel-miR-39) was added as the spike-in control to monitor extraction efficiency. The remainder of the RNA extraction was performed according to the manufacturer's instructions. miRNAs were eluted with 30 μl of RNase-free water. Total RNA, including miRNAs, from cells or tissues were isolated using TRIzol reagent (Invitrogen, Carlsbad, CA, USA). cDNA was synthesized using the Quanta Biosciences qScript™ cDNA Synthesis Kit (95047-100) for mRNA analysis, and using the qScript™ microRNA cDNA Synthesis Kit (95107-100) for miRNAs analysis. qRT-PCR of miRNAs and mRNA was performed using the ABI 7900 HT Real-Time PCR System and a SYBR Green PCR Kit: Quanta Cat. #84018 and #84071 respectively. The fold expression and statistical significance were calculated using the 2-ΔΔ Ct method. All experiments were performed in triplicates.

High Fat diet fed mice. C57BL/6 male mice were fed for 8 weeks a 50% high fat diet (Envigo, DIETTD150235). All mice were kept in a pathogen-free facility, under a 12 h light/dark cycle. Research on mice was approved by the Hebrew University Institutional Animal Care and Ethics Committee.

Compound 1 and AntagomiR injections to mice. C57BL/6 male mice, 7-8 weeks old, or Sgp130FC, 9-month-old male mice, were injected i.p. with 7.5 mg/kg Compound 1 dissolved in saline and 3% DMSO. Saline was injected as control. Mice were hydrodynamic tail vain injected with antagomiR-122 or antagomiR-control (negative control) (5 μg/mouse in 1.5 ml saline). Mice were sacrificed according to the legend of the figure describing the experimental results and the livers, white adipose and skeletal muscle tissues were frozen in liquid nitrogen or in OCT embedded frozen blocks, for further RNA and histologic analysis. AntagomiRs were obtained from Sigma Aldrich, see Table 3.

TABLE 2
Primers for plasmids constructs.

| | | | Restric- | |
|---|---|---|---|---|
| | | | tion | |
| | | | sites | |
| Primer | | Sequence | at | |
| Number | Direction | (5′ to 3′)^{ab} | 5′-end | Cloning |
| P1 | forward | GACACGCGTAG | MluI | Mutate |
| | | TCAACATGGTG | | RORα |
| | | AAACCC | | sites in |
| | | | | miR-122 |
| | | | | promoter |
| P2 | reverse | TATTGCTTTTT | SpeI | Mutate |
| | | ATTTTTTAACT | | RORα |
| | | AGTCCTTTTTT | | site |
| | | TGAAATGGA | | in miR- |
| | | | | 122 |
| | | | | promoter |

TABLE 3
Synthetic small RNA.

| Name | Sequence |
|---|---|
| antagomiR- | 5′-mAsmCsmAmAmAmCm |
| 122 | AmCmCmAmUmUmGmUmCm |
| | AmCmAmCmUsmCsmCsmA |
| | s-Chol-3′ |
| antagomiR- | 5′-mCsmAsmCmCmAmCm |
| control | AmUmAmCmCmGmCmAsmC |
| | smGsmGs-Chol-3′ |

Subscript ‘m’ represent 2′-O Me-modified nucleotides; Subscript ‘s’ represents a phosphorothioate linkage; ‘Chol’ represents cholesterol linked through a hydroxyprolinol linkage.

### Introduction

MicroRNA-122 (miR-122) is associated with FGF21 expression. MiR-122 expression is dependent on inflammation signaling, which also cases it secretion from the liver to have remote effects on other organs. Furthermore, miR-122 is also regulated by free fatty acids (FFA) mediated by the activation hepatocytes RORα. MiR-122 increase in hepatocytes by the FFA-RORα machinery, which results in the upregulation of FGF21.

A panel of RORα agonists was developed, and one compound was selected based on its effect on activating the miR-122 promotor. This activation caused beneficial increases in endogenous FGF21 levels, which, in turn, can treat pancreatitis.

### Results

### RORα Activation

RORα regulates miR-122 expression in mice, and this is mediated through FFA (Chai, Gastroenterology, Volume 153, Issue 5, November 2017, Pages 1404-1415). The levels of RORα decreased upon HFD, and increased upon activation with the RORα activator Compound 1.

The expression of miR-122 target genes is increased in humans in which miR-122 is decreased. MiR-122 target genes are also negatively correlated with RORα expression. The expression of FGF21 is positively correlated with pre-miR-122.

FGF21 is a known target of RORα (Wang, J Biol Chem. 2010 May 21; 285(21):15668-73). Recently, it was shown that FGF21 is upregulated in the liver upon cold exposure (Ameka, Sci Rep. 2019 Jan. 24; 9(1):630). While not wishing to be bound to a particular theory, it is believed that this could be due to an increase of RORα in the cold. To assess this assumption, HUH7 human HCC cells were transfected with a RORα reporter system in which luciferase is expressed from the miR-122 promoter that harbors a RORα binding site. The increase in primiR-122 and miR-122 levels is associated with a decrease in miR-122 target genes, Aldo A and Dgat1. MiR-122 expression is cold sensitive, depending on RORα binding to its consensus sequence in the miR-122 promotor. In an effort to determine whether the RORα-miR-122 machinery is relevant to humans, as well, a human study was conducted (Hadassah University Hospital IRB approval #HMO-0025-18). In this study, humans were undergoing major blood vessels cardiovascular surgery with the usage of the cardio-pulmonary machine and systemic body cooling. MiR-122 expression was measured, and a significant increase in plasma miR-122 was found upon temperature reduction.

These results suggested that an increase in RORα activity and an increase in the activity of the miR-122 promotor can increase the levels of FGF21 expression, which, in turn, have a beneficial effect on pancreatitis. To this end, a panel of RORα agonists was generated.

RORα is composed of an N-terminal activation function 1 (AF-1) that interacts with coactivator proteins followed by a DNA-binding domain containing two zinc-finger motifs, a flexible hinge region, and a C-terminal ligand binding domain (LBD) that contains a hormone-responsive activation function 2 (AF-2). The binding of an agonist to the RORα-LBD induces a conformational change that enables binding of coactivator proteins to the AF-2. The most potent agonist solved in complex with RORα-LBD is cholesterol sulfate (PDBID 1S0X). This ligand-binding pocket of this crystal structure was targeted by high throughput virtual screening to identify novel RORα agonists. A proprietary library of drug-like 300,000 compounds were evaluated for binding using the Schrodinger Maestro Glide HTVS workflow. The top 200 compounds were further scored using Prime MMGBSA with 5 Å flexibility allowed. The top 100 compounds were visually inspected, and twelve were selected for evaluation using the luciferase assay at the miR-122 promoter region.

RORα Liver and Systemic Effects are Mediated Through miR-122

The following experiments were conducted in an effort to determine whether the RORα metabolic and biochemical effects are mediated through miR-122. The activation by Compound 1 is through the RORα DNA binding/activation to the miR-122 promotor by mutating this site in the miR-122 promotor. Upon exposing HuH7 cell to Compound 1 for 16 h, cells levels of miR-122 did not change, but a significant miR-122 was secreted to the medium (there was no apparent toxicity to the cells as measured by LDH release, data not shown)). However, when Compound 1 was administered to mice for a number of weeks, miR-122 levels increased both in the liver and in the plasma. This was associated with an increase of hepatic precursor's levels of both Pre-miR-122 and Pri-miR-122, as well as a decrease in a known target of miR-122, AldoA and an increase in G6Pase, a known RORα target gene (Chauvet, PLoS One. 2011; 6(7):e22545). MiR-122 reaches remote tissues. To assess the effect of Compound 1 on this miR-122 remote effect, miR-122 was measured in heart muscle tissue, which showed an increase in the mature miR-122 levels with a reciprocal down-regulation of three miR-122 target genes. Mature miR-122 was also identified in other organs as WAT and muscle after administration of Compound 1 (the levels of pri-miR-122 in muscle tissue were not detected, suggesting that the mature miR-122 in the muscle was not expressed from the miR-122 promotor).

In an effort to determine whether the mechanism of action of miR-122 and Compound 1 are aligned at the same pathway, an experiment was designed in which both molecules, together and each separately, were administered. In this study, mice were fed with a 50% HFD and therapy was initiated 4 weeks after the animals were already on a diet, to establish NASH prior to treatment. Therapies (antagomiRs given once a week, due to a prolonged half time, and RS twice weekly, due to a plasma t_{1/2} of 2.7 hrs) were initiated after 4 weeks and given for 3 weeks. The mice were weighed from week 3 and therapies initiated a week later. Control mice (antagomiR-control once a week and DMSO diluted in saline twice a week) had a steady increase in weight. Mice in which an antagomir-122 was administered had the highest increase in weight. Those treated with the RORα agonist Compound 1, their weight steadily decreased and lost weight. Mice administered both, antagomir-122 and Compound 1, their weight returned exactly to that of controls animals. This phenomenon suggests that miR-122 and Compound 1 probably antagonize one another.

Liver and plasma reductions of miR-122 were observed upon administration of antagomiR-122, and an increase of miR-122 in both liver and plasma was observed when Compound 1 was administered. MiR-122 increase had also effects on its target genes including Agpat1, Dgat1 and FGF21. The liver antagomiR-122 and the Compound 1 had also an effect on muscle with a similar pattern to that in the liver, possibly through miR-122 secretion effects.

The level in the liver of FGF21 message is correlated associated with pri-miR-122 levels, suggesting a co-regulation. These observation strengths our hypothesis that miR-122 levels control, either by reducing hepatic miR-122 levels by antagomiR-122, or increase it, by Compound 1, has both a liver/central and remote/peripheral effects. The overall effects of Compound 1 culminated in enhanced FGF21 levels, which can be beneficial in treating, preventing reduce the susceptibility to, reducing the severity of, and/or delaying the progression of pancreatitis.

### Effects of Activating the RORα-miR-122-Triglycerides Circuitry

After showing that Compound 1 is a clinically relevant miR-122 activator with beneficial biochemical effect, the study next aimed to determine its effect on mir-122 precursors. Compound 1 was administered to mice with an established NASH. The activator increased both miR-122 levels in the livers of mice as well as in plasma. The administration of the RORα activator/agonist, Compound 1 compound, resulted in an increase in miR-122 precursors as well as in RORα targets. These results demonstrated that the activator was truly functioning in the model.

The Anti-Inflammatory and Anti-Fibrogenic Effects of Activating the RORα-miR-122-Triglycerides Circuitry with Compound 1

Once it was observed that the RORα activator Compound 1 had beneficial metabolic properties, the effects of Compound 1 on liver inflammation and fibrosis were determined. The effects of Compound 1 on liver inflammation and fibrosis were assessed in the mouse atherogenic diet model (Anavi, Lab Invest. 2015 August; 95(8):914-24). After liver inflammation and fibrosis developed at week 3 of diet, animals initiated to receive Compound 1. After 3.5 additional weeks, in which animals received 3 times weekly Compound 1, animals were assessed for numerous endpoints. Compound 1 significantly improved liver enzymes. It was confirmed that mature miR-122 increased both in tissue and plasma following the administration of Compound 1. Compound 1 significantly improved liver inflammation. This improvement in inflammation was associated with a significant reduction in liver fibrosis, as assessed by two measures, Masson Trichrome and αSMA staining. There was no effect of Compound 1 on liver vasculature as depicted by CD34 staining of these mice livers (data not shown). The effect of the RORα activator, Compound 1, was apparent also on fibrosis driver genes. FGF21 is also associated with inflammation and fibrosis in the pancreas, so the effects of Compound 1 on FGF21 levels, and the resulting effects on liver inflammation and fibrosis, can be extrapolated to the treatment of pancreatitis, as well as its prevention, reducing the susceptibility to pancreatitis, reducing the severity of pancreatitis, or delaying the progression of pancreatitis.

### Discussion

Activating RORα has major beneficial effects with respect to pancreatitis. The beneficial effect of RORα on pancreatitis is mediated through mature miR-122, although additional RORα activities could potentially contribute to these beneficial effects. The role of miR-122 is through targeting the expression of central enzymes in TG biosynthesis. Based on the evidence, it was shown that RORα activates, the expression of miR-122 and also increases its secretion into the plasma, to reach WAT, muscle and heart muscle, to expedite its remote effects, we propose that the effect of RORα activation is both in the liver and systemic.

In an effort to control the activation of RORα and enhance its potential beneficial effects, a screening system was developed to identify compounds that enhance RORα activity on miR-122 expression. We have identified a compound (Compound 1) which has potential therapeutic effects in enhancing FGF21 levels. Interestingly, Compound 1, which increased miR-122 expression and secretion, also showed significant metabolic effects, additionally demonstrating its usefulness in treating the subset of pancreatitis that caused by metabolic disorders.

A number of previous reports, and those of ours, pointed to the potential of “hijacking” the mechanistic action of miR-122 as an anti lipotoxic effector in the liver. The machinery of producing miR-122 in the liver is robust. Each hepatocyte stores 250,000 copies of miR-122 as well as miR-122* (Simerzin, Hepatology. 2016 November; 64(5):1623-1636). The effective remote activity of miR-122 is dependent on the high production and secretion of miR-122 to generate high plasma levels. This high production rate suggests that miR-122 could be translated into an effective therapeutic compound. However, rather than developing a system in which a synthetic miR-122 (mimic-miR-122) is synthesized, made as a drug and injected to patients with NASH for years, it would have been preferred to develop a small drug that induces the expression of the hepatic endogenous miR-122, and could be given daily to patients. MiR-122 is also expressed and secreted by TNFα signaling. However, injecting TNFα is not relevant in the clinical setting of NASH. MiR-122 also has additional therapeutic properties, including increasing FGF21 expression.

The data shown in this report proposes that RORα activation, which increases miR-122 both in the liver and reaches other organs, including the pancreas, has a substantial activity. RORα activators are therefore proposed as promising compounds to be developed and assess for their clinical beneficial effects on pancreatitis in patients.

### Materials and Methods

### Cell Culture

Human hepatocellular carcinoma cell line-Huh7 were cultured in DMEM supplemented with 10% fetal calf serum (FCS), 1% penicillin/streptomycin (Thermo Scientific, Waltham, MA, USA). Cells were cultured at 37° C. in a humidified atmosphere containing 5% CO₂, except for experiment in which cells were placed in 32° C. as indicated in the text.

### RNA Extraction and Quantitative Real Time RT-PCR

Total RNA, including small RNAs, were isolated from 200 μL of plasma or culture media samples using the miRNeasy Mini kit (Qiagen, Valencia, CA, USA) with 2 minor modifications. First, 200 μl of plasma or culture media were lysed with 1 ml of Qiazol solution. Second, a 50 pmol/l of synthesized single strand Caenorhabditis elegans miRNA (cel-miR-39) was added as the spike-in control to monitor extraction efficiency. The remainder of the RNA extraction was performed according to the manufacturer's instructions. miRNAs were eluted with 30 μl of RNase-free water. Total RNA, including miRNAs, from cells or tissues were isolated using TRIzol reagent (Invitrogen, Carlsbad, CA, USA). cDNA was synthesized using the Quanta Biosciences qScript™ cDNA Synthesis Kit (95047-100) for mRNA analysis and using the qScript™ microRNA cDNA Synthesis Kit (95107-100) for miRNAs analysis. qRT-PCR of miRNAs and mRNA was performed using the ABI 7900 HT Real-Time PCR System and a SYBR Green PCR Kit: Quanta Cat. #84018 and #84071 respectively. The fold expression and statistical significance were calculated using the 2-ΔΔ Ct method. All experiments were performed in triplicates. The primers used for qRT-PCR are shown in Table 4.

### Plasmids

The human miR-122 promoter fragments spanning the region from −900 bp relative to the transcription start site (TSS) and mutating the RORα binding site (plasmids PmiR-122-900 and PmiR-122-RORα mut, respectively) were generated as described previously.

### Transfections

For Luciferase assays, cells grown in 24 well plates were co-transfected with a luciferase reporter plasmid (50 ng) and 1 ng of Renilla Luciferase vector (PRL, Promega) with Lipofectamine LTX (Invitrogen) transfection reagent. For all experiments, the transfection performed using serum-free medium (Opti-MEM; Cat #31985070; Thermo Scientific).

### Luciferase Activity Assay

Following transfections, the cells were lysed with passive lysis buffer (Cat #E1941; Promega), shaking for 20 min at RT and transferred into appropriate 96-well plate. Firefly and Renilla luciferase activity was assessed using the Dual Luciferase Reporter Assay system (Cat #E1910; Promega) on a luminometer Mithras 2000 (Centro XZ, LB960, Berthold Technologies, Bad Wildbad, Germany). The luciferase activity was normalized to Renilla luciferase activity. Readings were taken in triplicate.

### RORα Agonist Treatments

Commercial RORα agonist SR1078 (Cayman Chemical) and RORα compounds stocks were prepared by dissolving in DMSO (1 mg/ml). Huh7 cells were treated overnight with 5 μM SR1078 or with 1 μM of all other tested compounds. DMSO alone (0.2%) was used as control. The RORα agonist, Compound 1 as dissolved in saline and up to 5% DMSO, and was injected i.p. to mice in the dosage according to the text. Triglycerides, free fatty acids and (3-hydroxybutyrate were quantified.

To determine the liver and muscle lipid content, muscle and liver tissues (40-80 mg) were homogenized in 0.5 ml of chloroform: Tris solution (v/v, 1:1), the homogenate was transferred to 1 ml of chloroform:methanol solution (v/v, 2:1) centrifuged at 3000 g (at −2° C.) for 10 min (Heraeus Megafuge 16R centrifuge). The organic phase was mixed with 5% Triton X100 in chloroform, dried and re-dissolved in water. After lipid extraction, triglyceride (TG) concentration in samples was measured with Triglyceride Quantification Kit (BioVision), according to the manufactures instructions. Plasma Free fatty acids and β-hydroxybutyrate were determined utilizing commercial colorimetric kits (BioVision) directly from plasma samples.

### Animal Studies

Male C57BL/6 mice, 7-8 weeks old, were purchased from Harlan Laboratories (Jerusalem, Israel). All mice were kept in a pathogen-free facility, under a 12 h light/dark cycle. Mice were handled according to the criteria outlined in the “Guide for the Care and Use of Laboratory Animals” prepared by the National Academy of Sciences and published by the National Institutes of Health. Research on mice was approved by the Hebrew University Institutional Animal Care and Ethics Committee; ethics number MD-15-14423-3.

### AntagomiR-122 Treatment of High Fat Diet (HFD) Fed Mice

C57BL/6 mice, 7 to 8 weeks old, were fed chow or 50% HFD, consisting of 50% Fat, 20% Sucrose, 10% Fructose, 1.25% Chol) (Envigo, TD.150235) for 4 weeks. In experiments of miR-122 repression by antagomiR, mice were hydrodynamic tail vain injected with antagomiR-122 or antagomiR-control (5 μg/mouse in 1.5 mL saline) once a week for 4 weeks and were still fed HFD or chow diet. After 4 weeks of injections mice were sacrificed and the livers, white adipose, and skeletal muscle tissues were frozen in liquid nitrogen or in optimum cutting temperature embedded frozen blocks, for further RNA and histologic analysis. AntagomiRs were obtained from Sigma-Aldrich (St Louis, MO); see Table 5.

### Compound 1 and AntoagomiR-122 Treatment of HFD or Atherogenic Diet-Fed Mice

Male C57BL/6J mice, 7 to 8 weeks old, were housed randomly in standard cages and were fed a HFD, or atherogenic diet (consisting 1% Chol and 0.5% cholic acid, see also Table 6). All mice had free access to water during the experimental period. During the feeding period body weight was monitored every 3 days. In the HFD experiment, after 4 or 6 weeks, the resultant obese mice were treated with antagomiR-122 (5 μg/mouse once a week for 3 weeks), or i.p. injected with Compound 1 (RORα agonist, 7.5 mg/kg twice a week for 3 weeks; or 15 mg/kg 3 times a week for 3 weeks). The obese control (HFD) group was administered only saline with DMSO and antagomiR-control. After 3 weeks of treatment mice were sacrificed and livers were taken for RNA-seq analysis. Livers, white adipose, and skeletal muscle tissues were frozen in liquid nitrogen or in optimum cutting temperature embedded frozen blocks, for further RNA and histologic analysis. In the atherogenic diet experiment mice were treated with 15 mg/kg Compound 1 after 3 weeks with the diet. After 3.5 weeks of treatment, mice were sacrificed, and the livers were frozen in liquid nitrogen or in optimum cutting temperature embedded frozen blocks. Plasma was collected from atherogenic diet-fed mice and saved in −20° C. for ALT and AST analysis using the Reflotron® Analyzer and test-strips (Roche).

### Multi-Parameter Metabolic Assessment

Metabolic and activity profiles of the mice were measured, by using the Promethion High-Definition Behavioral Phenotyping System (Sable Instruments, Inc., Las Vegas, NV, USA), which is a multi-parameter assessment incorporating sub-systems for open-circuit indirect calorimetry, feeding, water intake, activity, running wheel and body mass measurements in a conventional live-in home cage that minimizes stress. Data acquisition and instrument control were performed using the MetaScreen software version 2.2.18.0, and the obtained raw data were processed using ExpeData version 1.8.4 using an analysis script detailing all aspects of data transformation. C57BL/6 mice were fed for 6 weeks with HFD and then treated with 15 mg/kg Compound 1 3 times a week for 2 weeks, then were placed in metabolic chambers, with a free access to food and water and were subjected to a standard 12 h dark/12 h dark cycle, which consisted of a 24 h acclimation period followed by a 48 h sampling duration. Respiratory gases were measured by using the GA-3 gas analyzer (Sable Systems Inc., Las Vegas, NV, USA) using a pull-mode, negative-pressure system. Air flow was measured and controlled by the FR-8 (Sable Systems Inc., Las Vegas, NV, USA), with a set flow rate of 2000 mL/min. Water vapor was continuously measured and its dilution effect on O₂ and CO₂ were mathematically compensated. Effective mass was calculated by ANCOVA analysis. Respiratory quotient (RQ) was calculated as the ratio of VCO₂/VO₂. Total energy expenditure (TEE) was calculated as VO₂×(3.815+1.232×RQ), normalized to effective body mass, and expressed as kcal/h/kgeff·Mass. Fat oxidation (FO) and carbohydrate oxidation (CHO) were calculated as: FO=1.69×VO₂−1.69×VCO₂ and CHO=4.57×VCO₂−3.23×VO₂ and expressed as g/d/kgeff·Mass. Ambulatory activity and position were monitored simultaneously with the collection of the calorimetry data using the XYZ beam arrays with a beam spacing of 0.25 cm.

### Oil Red O Staining

Liver tissues were embedded in Optimal Cutting Temperature gel and cut into 10 m frozen sections. For Oil Red 0 staining, a stock solution of Oil Red 0 (Sigma-Aldrich) (1 g/10 mL in Propylene Glycol) was prepared, filtered, and protected from light. Frozen sections were dipped in formalin, stained with Oil Red 0 for 15 min, followed by counterstaining with hematoxylin for 30 sec.

### Human Blood Samples and Heparin Elimination

For the measurement of miR-122, FFA and human FGF21 (abeam) analysis in blood samples collected from patients undergoing major blood vessels cardiovascular surgery with the usage of the cardio-pulmonary machine and systemic body cooling. This was performed under the approval of the Hadassah Hospital IRB committee approval number 0025-18-HMO. Informed consent and permission to use biological materials for research were obtained from all subjects. Tube no. 2 indicates the time during the surgery before cooling the patient and Tube no 3. represents the time when the body temperature was the lowest during the surgery. Heparin elimination from RNA solutions isolated from plasma samples of patients was performed according to the protocol described priviously^{3,4}, briefly, a 5 μL RNA sample in water was mixed with 5 μL of heparinase working solution (0.085 IU/mL of Heparinase I (Sigma-Aldrich; catalogue no H2519), 2000 units/mL of RiboLock RNase Inhibitor (Life Technologies; catalogue no E00381), 10 mmol/L Tris HCl pH 7.5, 2 mmol/L CaCl₂, 25 mmol/L NaCl) and incubated at 25° C. for 3 h. After reaction the samples were directly used in reverse transcription reactions as RNA templates.

### Tissue Histology and Immunohistochemistry

Livers and adipose samples were placed in 4% buffered formaldehyde for 24 hours, followed by 80% ethanol and then embedded in paraffin blocks. Liver and adipose tissues were cut into 5 mm sections, deparaffinized with xylene and hydrated through graded ethanol. For the H&E staining, tissue sections were stained with hematoxylin (Emmonya Biotech Ltd.) and eosin (Leica, Surgipath). Liver macrophages were stained using rat anti-mouse F4/80 antigen (Serotec), followed by anti-Rat HRP (Histofine) and developed with a DAB kit (Zymed). Liver sections were stained for Masson Trichrome (Sigma). Liver CD3+ T cells were stained using Rat anti human-CD3-antibody (Bio-Rad), followed by anti-Rat HRP (Histofine) and developed with AEC (Invitrogen). α-SMA positive cells were stained using mouse anti-Human smooth Muscle Actin antibody (Dako), followed by anti-mouse HRP (Dako) and developed with DAB. The percentage area stained positively per high power field was calculated by ImageJ Software in 5-10 random fields.

### Statistical Analysis

Data were subjected to statistical analysis using the Excel software package (Microsoft, Redmond, WA) or GraphPad Prism6 (GraphPad Software Inc., La Jolla, CA). Two-tailed Student t tests, and Pearson and Spearman correlation coefficients were used to determine the difference between the groups. Data are given as mean±SD, and are shown as error bars for all experiments. Differences were considered significant at P<0.05. The reported data were obtained from at least 3 biological replicates.

TABLE 4
Primers used for Real-Time-PCR

| Primer | | |
|---|---|---|
| Number | Direction | Sequence (5′ to 3′) |
| Mouse DGAT1 | Forward | GTGGTTTCAGCAATTATCGTGG |
| | Reverse | GGGTCCTTCAGAAACAGAGAC |
| Mouse HPRT | Forward | GCGATGATGAACCAGGTTATGA |
| | Reverse | ATCTCGAGCAAGTCTTTCAGTC |
| | | CT |
| Mouse pri- | Forward | TTCGGGAACTATGTGGAGTCACT |
| miR-122 | | TTG |
| | Reverse | CGCAAGGCTGCCCTCAAACCCTC |
| | | AG |
| Mouse pre | Forward | CCATCAAACGCCATTATCACACT |
| | | A |
| -miR-122 | Reverse | CACACAATGGAGAACTCTAGCAC |
| | | AA |
| Mouse ALDOA | Forward | GGGTGATCCTCTTCCACGAGA |
| | Reverse | AGGGGCACCACACCCTTATC |
| Mouse AGPAT1 | Forward | GGGCGCAATGTCGAGAACATG |
| | Reverse | CTGGCAGGACCTCCATCATTC |
| Mouse CPT1α | Forward | AGACAAGAACCCCAACATCC |
| | Reverse | CAAAGGTGTCAAATGGGAAGG |
| Mouse TGFBR2 | Forward | CAAGTTTTGCGATGTGAGACTG |
| | Reverse | CCGTCTCCAGAGTAATGTTCTTG |
| Mouse TGFB2 | Forward | CTCTGTGGGTACCTTGATGCC |
| | Reverse | GGAAGACCCTGAACTCTGCC |
| Mouse FGF21 | Forward | CAAATCCTGGGTGTCAAAGC |
| | Reverse | CATGGGCTTCAGACTGGTAC |
| Mouse IFNβ | Forward | TCCGAGCAGAGATCTTCAGGAA |
| | Reverse | TGCAACCACCACTCATTCTGAG |
| mouse ACTA2 | Forward | GTGAAGAGGAAGACAGCACAG |
| (alpha sma) | Reverse | GCCCATTCCAACCATTACTCC |
| Mouse RORα | Forward | CATGGTTCCTAAGGGATGAGAG |
| | Reverse | CATGGTTCCTAAGGGATGAGAG |
| Mouse | Forward | CCCCAGTGTCCTTACAGAGTG |
| ADGRE1 | Reverse | GTGCCCAGAGTGGATGTCT |
| (F4/80) | | |
| Mouse | Forward | CATAAAGGGTCATCGTGGCT |
| COLA1 | Reverse | TTGAGTCCGTCTTTGCCAG |
| Mouse | Forward | GAAGTCTCTGAAGCTGATGGG |
| COL3A1 | Reverse | TTGCCTTGCGTGTTTGATATTC |
| Mouse | Forward | TCACTTCTACTCTTGCTATCT |
| G6PC | | TTCG |
| | Reverse | CCCAGAATCCCAACCACAAG |
| Mouse | Forward | CTGTAGCCCACGTCGTAGCAA |
| TNFα | Reverse | CTGGCACCACTAGTTGGTTGT |
| miR-122 | Forward | TGGAGTGTGACAATGGTGTTTG |
| miR-34 | Forward | TGGCAGTGTCTTAGCTGGTTGT |
| miR-18a | Forward | TAAGGTGCATCTAGTGCAGATAG |
| miR-21 | Forward | TAGCTTATCAGACTGATGTTGA |
| miR-126-5p | Forward | CATTATTACTTTTGGTACGCG |
| miR-93-5p | Forward | CAAAGTGCTGTTCGTGCAGGTAG |
| RNU-6 | Forward | CGCAAGGATGACACGCAAATTC |
| Cel-miR- | Forward | TCACCGGGTGTAAATCAGCTTG |
| 39-3P | | |

TABLE 5
antagomiR sequences used in the study.
All the oligonucleotides were synthesized
by IDT (IDT, Coralville, IA, USA).
Chemical modifications of the antisense
oligos: Subscript ‘m’ represent
2′-O Me-modified nucleotides;
Subscript ‘s’ represents a
phosphorothioate linkage; ′Chol′
represents cholesterol linked through
a hydroxyprolinol linkage.

| Name | Sequence |
|---|---|
| antagomiR- | 5′-mAsmCsmAmAmAmCmAmCmCm |
| 122 | AmUmUmGmUmCmAmCmAmCmUsm |
| | CsmCsmAs-Chol-3′ |
| antagomiR- | 5′-mCsmAsmCmCmAmCmAmUmAm |
| control | CmCmGmCmAsmCsmGsmGs- |
| | Chol-3′ |

TABLE 6
Normal and Atherogenic diet compositions.

| | Normal Diet | Atherogenic |
|---|---|---|
| | (gr/kg) | Diet (gr/kg) |
| cornstarch | 397.5 | 397.5 |
| maltodextrin | 132.0 | 132.0 |
| sucrose | 100.0 | 100.0 |
| casein | 200.0 | 200.0 |
| soybean oil | 70.0 | 70.0 |
| cellulose | 50.0 | 50.0 |
| mineral mix | 35.0 | 35.0 |
| vitamin mix | 10.0 | 10.0 |
| L-methionine | 3.0 | 3.0 |
| Choline | 2.5 | 2.5 |
| bitartrate | | |
| BTH | 0.014 | 0.014 |
| Cholesterol | 0.0 | 10.0 |
| Cholic Acid | 0.0 | 5.0 |
| total weight | 1000.0 | 1015.0 |

### REFERENCES

1. Chai, C. et al. Metabolic Circuit Involving Free Fatty Acids, microRNA 122, and Triglyceride Synthesis in Liver and Muscle Tissues. Gastroenterology 153, 1404-1415 (2017).
2. Rivkin, M. et al. Inflammation-Induced Expression and Secretion of MicroRNA 122 Leads to Reduced Blood Levels of Kidney-derived Erythropoietin and Anemia. Gastroenterology (2016). doi:10.1053/j.gastro.2016.07.031
3. Kondratov, K. et al. Heparinase treatment of heparin-contaminated plasma from coronary artery bypass grafting patients enables reliable quantification of microRNAs. Biomol. Detect. Quantif. 8, 9-14 (2016).
4. Izraeli, S., Pfleiderer, C. & Lion, T. Detection of gene expression by PCR amplification of RNA derived from frozen heparinized whole blood. Nucleic Acids Res. 19, 6051 (1991).

### Example 8

Demonstration that Compound 1 Acts as a Potent RORα Agonist and Increases FGF21 Expression

Experimental design: C57BL/6 mice fed for 6 weeks with high fat diet (HFD) were injected with 15 mg/kg Compound 1 (or saline+DMSO) 3 times a week for 3 weeks (n=6).

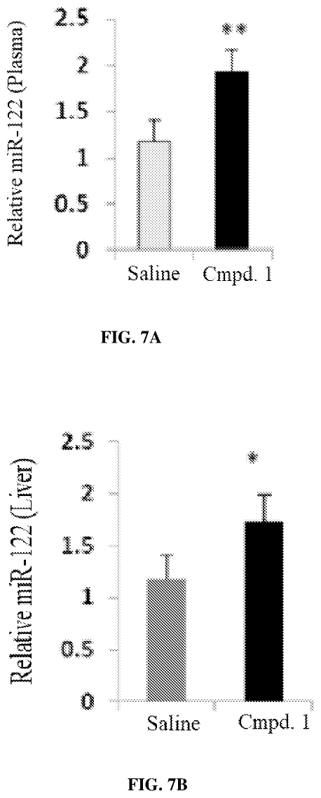

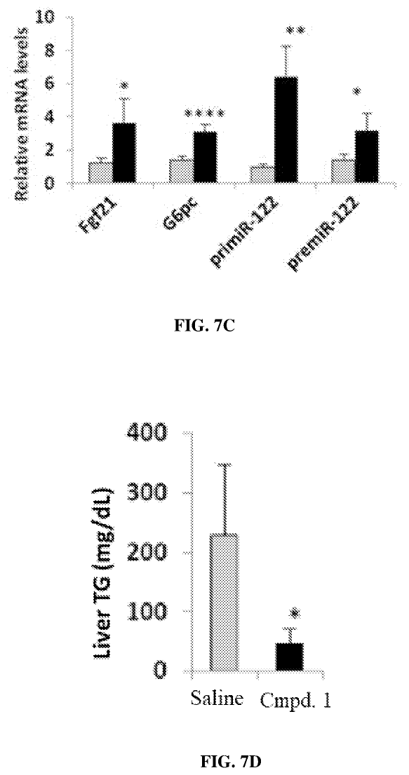

FIGS. 7 A and B show the results of qRT-PCR analysis of miR-122 extracted from plasma and liver, respectively, in mice treated with Compound 1 or saline. FIG. 7C shows the qRT-PCR analysis of RORα target genes, pri- and pre-miR-122 mRNA, extracted from mice livers.

Treatment with Compound 1 induced expression and secretion of miR-122 and precursors in the plasma and liver. Additionally, treatment with Compound 1 significantly induced expression of RORα-regulated genes FGF21 and Gpase6.

### Example 9

### The RORα Agonist, Compound 1, Improves Markers of Liver Damage and Fibrosis in a Fibrotic Diet Mouse Model.

Experimental design: C57BL/6 mice fed for 3 weeks with atherogenic diet (to induce fibrosis) and injected with 15 mg/kg Compound 1 (or saline+DMSO) 3 times a week for 3.5 week (n=8).

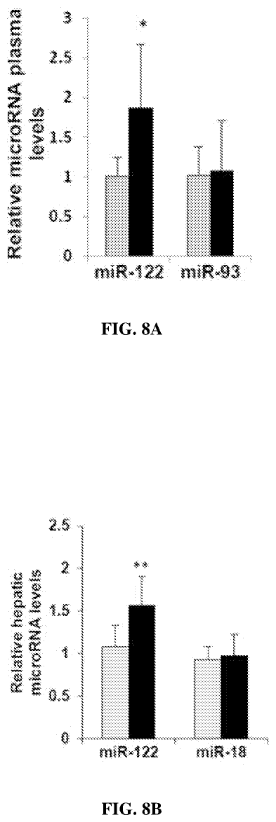

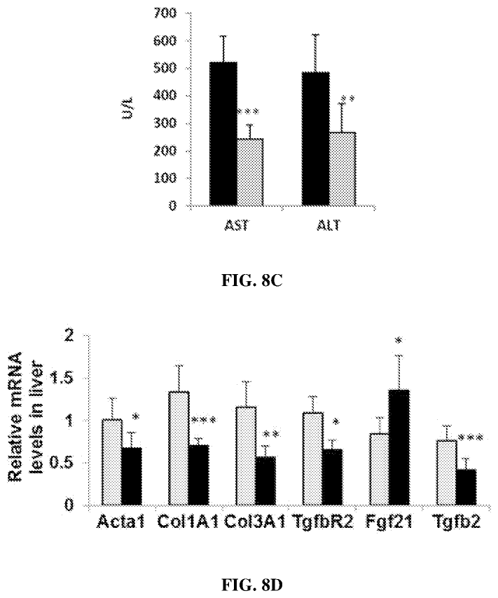

The results are shown in FIGS. 8A-D. qRT-PCR analysis of miR-122 extracted from 8A) plasma and 8B) from liver for the untreated (grey bars) and treated (black bars) cohorts. miR-93 and miR-18 were included for negative controls in plasma and liver, respectively. 8C) ALT and AST plasma levels measured at the end of the experiment. 8D) qRT-PCR analysis of mRNA of genes involved in fibrosis and RORα target gene (FGF21) extracted from mice livers. microRNA levels in the plasma were normalized to spiked C. elegans miR-39; microRNA levels in the tissues were normalized to RNU6. mRNA levels were normalized to HPRT. Data are presented as error bars=SD. *P<0.05, **P<0.01. ***P<0.001, ****P<0.0001.

The effects of Compound 1 on liver inflammation and fibrosis were determined. The effects of Compound 1 on liver inflammation and fibrosis in the mouse atherogenic diet model have been assessed. After liver inflammation and fibrosis developed at week 3 of diet, animals initiated to receive Compound 1. After 3.5 additional weeks, in which animals received 3 times weekly Compound 1, animals were assessed for numerous endpoints. We confirmed that mature miR-122 increased both in tissue and in plasma following the administration of Compound 1. Treatment with Compound 1 significantly improved biomarkers of liver injury, AST and ALT, in addition to reducing biomarkers of inflammation (Tgfb2 and TgfbR2) and fibrosis (Acta1, Col1A1 and Col3A1).

### Example 10

### The RORα Agonist, Compound 1, Improves Hepatic Inflammatory Profiles in a Fibrotic Diet Mouse Model.

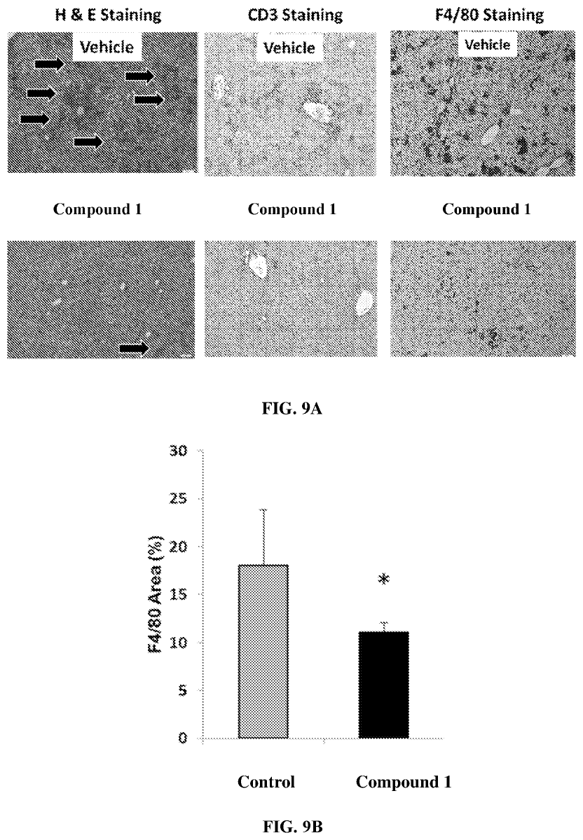

Experimental design: C57BL/6 mice fed for 3 weeks with atherogenic diet (to induce fibrosis) and injected with 15 mg/kg Compound 1 (or saline+DMSO) 3 times a week for 3.5 week (n=8). Representative microphotographs of H&E, CD3, and F4/80-stained livers taken from saline or Compound 1-treated mice are shown in FIG. 9A, where scale bars represent 10 μm. The graphs shown in FIG. 9B show quantification of positively-stained F4/80 areas using ImageJ.

Compound 1-treated mice showed decreased immune infiltrate by H&E staining, decreased T-cell density by CD3 staining and decreased levels of myeloid infiltrate by F4/80 staining. These results demonstrated that Compound 1 exhibits anti-inflammatory effects.

### Example 11

### The RORα Agonist, Compound 1, Decreases Hepatic Fibrosis in a Fibrotic Diet Mouse Model.

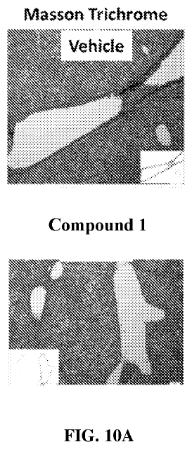

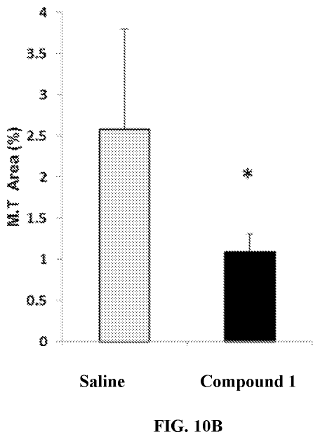

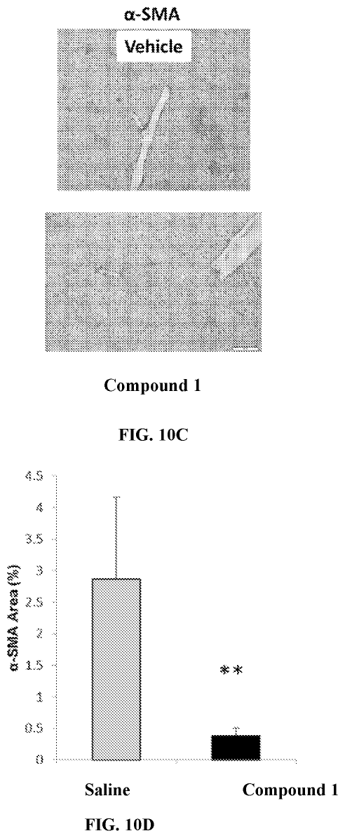

Experimental design: C57BL/6 mice fed for 3 weeks with atherogenic diet (to induce fibrosis) and injected with 15 mg/kg Compound 1 (or saline+DMSO) 3 times a week for 3.5 week (n=8). Results are shown in FIGS. 10A-D.

FIGS. 10A and C are representative microphotographs of Masson Trichrome (M.T.) and α-SMA stained livers taken from saline or Compound 1-treated mice, where scale bars represent 10 μm. FIGS. 10B and 10D are graphs showing the quantification of positively-stained areas using ImageJ.

Experimental design: C57BL/6 mice fed for 3 weeks with atherogenic diet (to induce fibrosis) and injected with 15 mg/kg Compound 1 (or saline+DMSO) 3 times a week for 3.5 week (n=8). Results are shown in FIGS. 10A-D.

Two stains were utilized to evaluate the effects of Compound 1 on liver fibrosis (Masson Trichrome and α-SMA). The untreated cohort exhibited large positive areas using both staining methods, and treatment with compound 1 significantly reduced the fibrotic areas by 5-fold (M.T) and 7-fold (α-SMA). These observations strongly support that Compound 1 exhibits anti-fibrotic activity in this mouse model.

### Example 12

Agonist of RORα Increases Promoter microRNA 122 (MIR122) Activity and Fibroblast Growth Factor 21 (FGF 21) Expression.

The following example was performed to show that an identified RORA (ROR-α) agonist increased the expression of MIR122 promoter activity and expression of FGF21. FGF21 itself can be used to treat pancreatitis (Hernandez et al., Sci. Transl. Med. 12, eaay5186 (2020). However, FGF21 is administered by injection, and it would be desirable to identify small molecules that can be orally administered, and increase endogenous levels of FGF21, rather than relying on injecting FGF21, particularly for chronic administration.

As discussed in this example, compounds have been identified that are RORα agonists, and these compounds increase MIR122 expression, as well as FGF21 expression. Thus, the compounds increase endogenous production of FGF21, and can be used to treat pancreatitis.

METHODS: A chemical library was screened to identify agonists of RORα. The effects of these compounds were evaluated on a human hepatocellular carcinoma cell line (Huh7). C57BL/6 mice were fed a chow or high-fat diet for 4 weeks to induce fatty liver. Mice were given hydrodynamic tail vein injections of a MIR122 antagonist (antagomiR-122) or a control antagomiR once each week for 3 weeks while still on the HFD or chow diet, or intraperitoneal injections of the RORα agonist Compound lor vehicle, twice each week for 3 weeks. Livers, gonad white adipose, and skeletal muscle were collected and analyzed by RT-PCR, histology and immunohistochemistry.

A separate group of mice were fed an atherogenic diet, with or without injections of Compound 1, for 3 weeks. Compound 1 has the following formula:

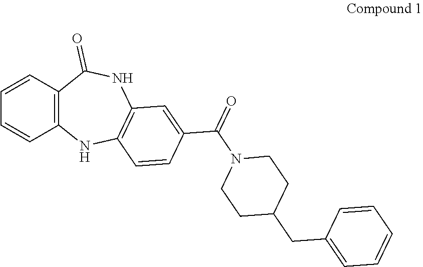 [SMILES: O=C1Nc2cc(C(=O)N3CCC(Cc4ccccc4)CC3)ccc2Nc2ccccc21]

Livers were analyzed by immunohistochemistry and plasma was analyzed for levels of aminotransferases. We analyzed data from liver tissues from patients with NASH included in the RNAseq databases GSE33814 and GSE89632.

RESULTS: Injection of mice with antagomiR-122 significantly reduced levels of MIR122 in plasma, liver, and white adipose tissue.

Compound 1 was identified as an RORα agonist, and found to increase expression of MIR122 promoter activity in Huh7 cells. In mice fed a HFD or atherogenic diet, injections of Compound 1 increased hepatic levels of MIR122 precursors and reduced hepatic synthesis of triglycerides, by reducing expression of biosynthesis enzymes.

Patients who underwent cardiovascular surgeries had increased levels of plasma MIR122 compared to its levels before the surgeries; increased expression of plasma MIR122 was associated with increased levels of RORα.

CONCLUSIONS: Compound 1 is an agonist of RORα that increases expression of MIR122 in cell lines and livers of mice. Agonists of RORα can be developed for treating pancreatitis, as well as other disorders mediated by FGF21.

### Introduction

The liver-specific microRNA-122 (MIR122) is associated with hepatic lipid metabolism. MIR122 is induced by free fatty acids (FFAs), and this induction is mediated by the activation of hepatic RORα. The increase in hepatic MIR122 by the FFA-RORα machinery, leads to suppression of triglycerides (TG) due to targeting and reducing the levels of enzymes involved in TG biosynthesis by MIR1228.

These and other effects caused by increase in MIR122 led to determining whether activating RORα could have other beneficial effects. Towards this aim, we engineered a panel of new RORα agonists, and selected one compound based on its significant effect of activating the MIR122 promotor. This activation caused beneficial effects in the livers of mouse models, including reversed fibrosis.

### Materials and Methods

### Cell Culture

The human hepatocellular carcinoma cell line Huh7 was cultured in DMEM supplemented with 10% fetal calf serum (FCS), 1% penicillin/streptomycin (Thermo Scientific, Waltham, MA, USA). Cells were cultured at 37° C. in a humidified atmosphere containing 5% CO₂, except for experiment in which cells were placed in 32° C. as indicated in the text.

### Plasmids

The human MIR122 promoter fragments spanning the region from −900 bp relative to the transcription start site (TSS) and mutating the RORα binding site (plasmids pMIR122-900 and pMIR122-RORα mut, respectively) were generated as described previously (Chai C. et al. Metabolic Circuit Involving Free Fatty Acids, microRNA 122, and Triglyceride Synthesis in Liver and Muscle Tissues. Gastroenterology 153, 1404-1415 (2017) and Rivkin M. et al. Inflammation-Induced Expression and Secretion of MicroRNA 122 Leads to Reduced Blood Levels of Kidney-Derived Erythropoietin and Anemia. Gastroenterology 151, 999-1010 e3 (2016).

### Transfections

For Luciferase assays, cells grown in 24 well plates were co-transfected with a luciferase reporter plasmid (50 ng) and 1 ng of Renilla Luciferase vector (PRL, Promega) with Lipofectamine LTX (Invitrogen) transfection reagent. For all experiments, the transfection performed using serum-free medium (Opti-MEM; Cat #31985070; Thermo Scientific).

### Luciferase Activity Assay

Following transfections, the cells were lysed with passive lysis buffer (Cat #E1941; Promega), shaking for 20 min at room temperature (RT) and transferred into the appropriate 96-well plate. Firefly and Renilla luciferase activity was assessed using the Dual Luciferase Reporter Assay system (Cat #E1910; Promega) on a luminometer Mithras 2000 (Centro XZ, LB960, Berthold Technologies, Bad Wildbad, Germany). The luciferase activity was normalized to Renilla luciferase activity. Readings were taken in triplicates.

### RORα Agonist Treatments

Commercial RORα agonist SR1078 (Cayman Chemical) and our newly synthesized RORα compounds stocks were prepared by dissolving in DMSO (1 mg/ml). Huh7 cells were treated overnight with 10 μM SR1078 or with 1 μM of all other tested compounds. DMSO alone (0.2%) was used as control. The RORα agonist, Compound 1 was dissolved in saline and up to 5% DMSO, and was injected i.p. to mice in the dosage according to the text.

RNA Extraction and Quantitative Real Time RT-PCR (qRT-PCR)

Total RNA, including small RNAs, were isolated from 200 μL of plasma or culture media samples using the miRNeasy Mini kit (Qiagen, Valencia, CA, USA) with 2 minor modifications. First, 200 μL of plasma or culture media were lysed with 1 ml of Qiazol solution. Second, a 50 pmol/L of synthesized single strand Caenorhabditis elegans miRNA (C. elegans miR-39) was added as the spike-in control to monitor extraction efficiency. The remainder of the RNA extraction was performed according to the manufacturer's instructions. miRNAs were eluted with 30 L of RNase-free water.

Total RNA, including miRNAs, from cells or tissues were isolated using TRIzol reagent (Invitrogen, Carlsbad, CA, USA). cDNA was synthesized using the Quanta Biosciences qScript™ cDNA Synthesis Kit (95047-100) for mRNA analysis and using the qScript™ microRNA cDNA Synthesis Kit (95107-100) for miRNAs analysis. qRT-PCR of miRNAs and mRNA was performed using the ABI 7900 HT Real-Time PCR System and a SYBR Green PCR Kit: Quanta Cat. #84018 and #84071 respectively. The fold expression and statistical significance were calculated using the 2-ΔΔ Ct method. All samples from one experiment were performed in triplicates.

### Animal Studies

Male C57BL/6 mice, 7-8 weeks old, were purchased from Harlan Laboratories (Jerusalem, Israel). All mice were kept in a pathogen-free facility, under a 12 h light/dark cycle. Mice were handled according to the criteria outlined in the “Guide for the Care and Use of Laboratory Animals” prepared by the National Academy of Sciences and published by the National Institutes of Health. Research on mice was approved by the Hebrew University Institutional Animal Care and Ethics Committee; ethics number MD-15-14423-3.

### AntagoMIR122 Treatment of High Fat Diet (HFD) Fed Mice

C57BL/6 mice, 7 to 8 weeks old, were fed chow or 50% HFD, consisting of 50% Fat, 20% Sucrose, 10% Fructose, 1.25% Cholesterol) (Envigo, TD.150235) for 4 weeks. In experiments of MIR122 repression by antagomiR, mice were hydrodynamic tail vain injected with antagoMIR122 or antagomiR-control (5 μg/mouse in 1.5 mL saline) once a week for 3 weeks and were still fed HFD or chow diet. After 3 weeks of injections mice were sacrificed and the livers, gonadal white adipose, and skeletal muscle tissues were frozen in liquid nitrogen or in optimum cutting temperature embedded frozen blocks, for further RNA and histologic analysis. AntagomiRs were obtained from Sigma-Aldrich (St Louis, MO).

### Compound 1 and AntoagoMIR122 Treatment of HFD or Atherogenic Diet-Fed Mice

Male C57BL/6J mice, 7 to 8 weeks old, were housed randomly in standard cages and were fed a HFD, or atherogenic diet (consisting of 1% Chol and 0.5% cholic acid. All mice had free access to water during the experimental period. During the feeding period, body weight was monitored every 3 days.

In the HFD experiment, after 4 or 6 weeks, the resultant obese mice were treated with antagoMIR122 (5 μg/mouse once a week for 3 weeks), or i.p. injected with Compound 1 (RORα agonist, 7.5 mg/kg twice a week for 3 weeks; or 15 mg/kg 3 times a week for 3 weeks). The obese control (HFD) group was administered only saline with DMSO and antagomiR-control.

After 3 weeks of treatment mice were sacrificed and livers were taken for RNA-seq analysis. Livers, gonadal white adipose, and skeletal muscle tissues were frozen in liquid nitrogen or in optimum cutting temperature embedded frozen blocks, for further RNA and histologic analysis. In the atherogenic diet experiment mice were treated with 15 mg/kg Compound 1 after 3 weeks with the diet. After 3.5 weeks of treatment, mice were sacrificed, and the livers were frozen in liquid nitrogen or in optimum cutting temperature embedded frozen blocks. Plasma was collected from atherogenic diet-fed mice and stored at −20° C. for ALT and AST analysis using the Reflotron® Analyzer and test-strips (Roche).

### Statistical Analysis

Data were subjected to statistical analysis using the Excel software package (Microsoft, Redmond, WA) or GraphPad Prism6 (GraphPad Software Inc., La Jolla, CA). Two-tailed Student t-tests, and Pearson and Spearman correlation coefficients were used to determine the difference between the groups. Data are given as mean±SD, and are shown as error bars for all experiments. Differences were considered significant at P<0.05. The reported data were obtained from at least 3 biological replicates.

### Results

the Effect of MIR122 on Lipid Metabolism in Mice Fed with a High Fat Diet (HFD)

Initially, we were interested in studying the function of MIR122 in livers of mice under HFD that cause lipotoxicity, since, in humans, MIR122 levels in livers with NASH are significantly lower.

MIR122 reduces TG accumulation by targeting the AGPAT1 and DGAT1 enzymes in the TG biosynthesis pathway. To test the effect of reducing MIR122 in livers of HFD-fed mice, we administered to the liver by a hydrodynamic tail-vein injection, antagoMIR122 that blocks and degrades MIR122 in hepatocytes. This injection caused the reduction of mature MIR122 levels in the livers of mice fed with normal diet (ND) and also in HFD mice. The levels of MIR122 precursors, pri- and pre-MIR122, were also reduced. Furthermore, antagoMIR122 injection reduced significantly the plasma level of MIR122 compared to miR-93 which was not affected. AntagoMIR122 injection also reduced MIR122 levels in the remote white adipose tissue (WAT). As opposed to MIR122, the level of miR-126 in WAT was reduced in HFD mice compared to ND and reducing MIR122 levels by antagoMIR122 caused a small, non-significant, increase in miR-126 levels.

Reduction of mature MIR122 levels in WAT as well as in muscle is a result of reduced secretion of MIR122 from hepatocytes, and not due to reduced MIR122 expression in non-liver tissues. The reduction of plasma MIR122 levels following antagoMIR122 injection, was associated with an increase of liver fat droplets and total TG liver content as well as an increase in muscle TG levels. The biochemical effect of liver MIR122 reduction was manifested by a decrease in oxidation (reduced plasma levels of β-hydroxybutyrate), as well as decreased liver Cpt1α levels (carnitine palmitoyltransferase 1A) an important enzyme in the α-oxidation pathway and a reduced plasma level of Free Fatty Acids (FFA).

All these are known indication of an increase in TG storage in tissues and reduced energy expenditure. Blocking of MIR122 by antagoMIR122, had an overall effect on mice weight that increased significantly in HFD mice. Liver weight also increased as well as the liver to body weight index. The effects of reducing MIR122 in the liver and systemically in remote tissues, lead to an increase in liver lipids, decrease in β-oxidation and energy expenditure, and have a systemic influence simulating altogether features of the metabolic syndrome.

Positive Correlation of the Expression of FGF21 (Fibroblast Growth Factor 21), a Known Target of RORα, with Pre-MIR122 Levels

In mice, RORα regulates MIR122 expression and this is mediated through FFAs8 (Data not shown). To study the potential relevance to human metabolism and NASH, we initially investigated human NASH data sets (GSE33814 and GSE89632 respectively). As discussed above, RORα is reduced in NASH patients. Furthermore, RORα target genes (ArgI and CD36) are decreased in these samples, and their expression is positively correlated with RORα. On the other hand, the expression of genes that are involved in FFA's biosynthesis pathway and are associated with fatty liver (Fasn and Srebf1), are negatively correlated with RORα.

Similarly, MIR122 target genes (AldoA, ADAM17 and Agpat1) are also negatively correlated with RORα expression and their level increases in human livers upon decreased RORα levels.

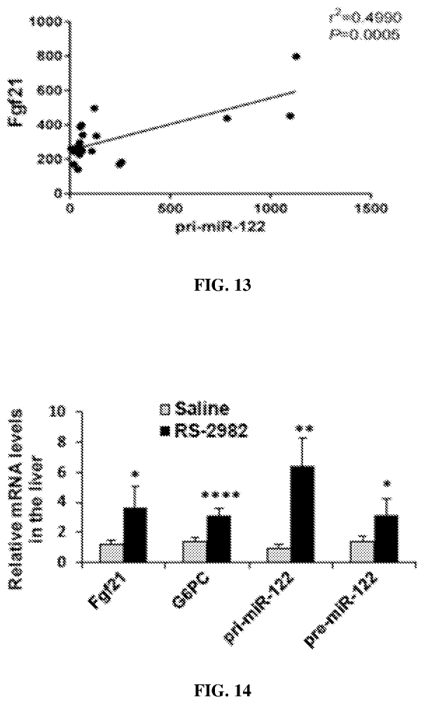

Importantly, the expression of FGF21 (fibroblast growth factor 21), a known target of RORα, is positively correlated with pre-MIR122 levels, suggesting co-regulation (FIG. 14).

Liver FGF21 is also upregulated upon cold exposure. While not wishing to be bound to a particular theory, this may be due to increased RORα levels in the cold. To assess this assumption, we transfected Huh7 human HCC cells with a RORα reporter plasmid expressing luciferase from the MIR122 promoter that harbors a RORα binding site.

MIR122 expression increased when the temperature was reduced to 32° C. (data not shown). When the RORα consensus binding sequence in the MIR122 promotor was mutated, the increase in MIR122 expression was abolished. In addition, MIR122 levels and its activity on target genes, increased in response to cold exposure of Huh7 cells.

To investigate further the RORα-MIR122 circuitry in humans, a human study was conducted (Hadassah University Hospital IRB approval #HMO-0025-18) where MIR122 plasma levels were measured in humans undergoing major blood vessels cardiovascular surgery with the usage of the cardio-pulmonary machine and systemic body cooling (data not shown).

A significant increase in plasma MIR122 levels was observed upon temperature reduction, which positively correlated with increased plasma FFAs levels.

### The Effect of a Newly Identified RORα Agonist Compound 1 on Liver and Systemic MIR122 Levels in Mice

The results shown above verify the FFAs-MIR122-TGs metabolic circuit. Thus, increasing RORα activity will lead to increased hepatic MIR122 expression, which further results in increased FGF21 expression. To this end, we sought to identify novel RORα agonists.

A targeted virtual screen was utilized to identify novel RORα agonists. A set of 300,000 drug-like commercially available compounds were docked and scored into the crystal structure of the RORα ligand-binding domain complexed with cholesterol sulfate. A final set of 10 compounds was selected for activity testing. This set of compounds was assayed for their induction of the MIR122 promoter using a luciferase promoter reporter plasmid (FIG. 3A). Notably, Compound 1 was the most potent at inducing MIR122 promoter, more potent than the commercial synthetic RORα agonist SR1078. SR1078 has the following structure:

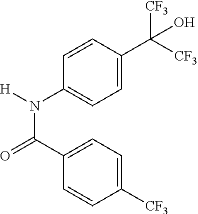 [SMILES: O=C(Nc1ccc(C(O)(C(F)(F)F)C(F)(F)F)cc1)c1ccc(C(F)(F)F)cc1]

The toxicity profile of Compound 1 was determined using a panel of cell lines. Compared to cycloheximide (+ control), Compound 1 had only moderate toxicity in CEM and Huh-7 (CC₅₀=11.0 and 10.4 μM, respectively) (data not shown). The chemical identity of this agent was confirmed by re-synthesis.

Compound 1 was further tested in a follow-up assay in which the RORα DNA response element in the MIR122 promoter was mutated. Consistent with RORα-specific activity, Compound 1 exhibited no induction of the mutated MIR122 promoter as opposed to the wild type promoter. When Huh7 cells were exposed to Compound 1 for 16 hours, cellular MIR122 levels did not change, but a significant amount of MIR122 was secreted to the medium. In addition, there was no apparent toxicity to the cells as measured by LDH release (data not shown). Importantly, treatment with Compound 1 increases MIR122 promoter activity in mice, and it is mediated by RORα, as a MIR122 reporter plasmid carrying a mutation in the RORα binding site, exhibited reduced promoter activity compared to the wild type (wt) promoter.

To demonstrate further that Compound 1 is a potent inducer in mice, MIR122 levels were monitored over time in mice after a single administration of the compound. MIR122 levels increased in the liver and plasma, and was associated with an increase in the levels of hepatic MIR122 precursor's (pri-MIR122 and pre-MIR122), as well as a decrease of a known target of MIR122, AldoA, and an increase in G6Pase, a known RORα target gene (Chauvet C. et al. Control of gene expression by the retinoic acid-related orphan receptor alpha in HepG2 human hepatoma cells. PLoS One 6, e22545 (2011)).

Following a single administration of Compound 1, mature MIR122 levels increased significantly in WAT, muscle and heart tissues. Furthermore, in the heart tissue, three MIR122 target genes were significantly down-regulated. These data demonstrate that Compound 1 is a potent inducer of MIR122 in mice.

Compound 1 reduces body weight and steatosis via increased MIR122 expression in HFD-fed mice.

Steatohepatitis in humans is correlated with reduced RORα levels. In accordance, mice fed with HFD had a temporal stepwise reduction in hepatic RORα levels. Interestingly, addition of the Compound 1 agonist increased the RORα level to its level in mice fed normal diet.

In line with our findings that Compound 1 increases MIR122 levels, we investigated further the mechanism of action of MIR122 and Compound 1 on NAFLD to learn whether they are aligned at the same pathway. We therefore designed an experiment in which Compound 1 was injected together with the MIR122 inhibitor, antagoMIR122, to mice with NAFLD.

Mice were fed with a 50% HFD and after 4 weeks, when fatty liver was already established, they were treated with Compound 1, antagoMIR122 or both compounds together for an additional 3 weeks. AntagoMIR122 was given once a week, due to a prolonged half life time, and Compound 1 twice weekly, due to a plasma t_{1/2} of 2.7 hr's. The weighing of mice began at week 3, one week before treatments were initiated. Control mice (antagomiR-control once a week and DMSO diluted in saline twice a week) had a steady increase in body weight however, mice administered antagoMIR122, had the highest increase in weight. In contrast, mice treated with the RORα agonist Compound 1, exhibited a significant decrease in body weight. The weight of mice administered both, antagoMIR122 and Compound 1, returned exactly to that of control animals. Upon cessation of the experiment, there was a significant increase in body weight in the antagoMIR122 treated animals, indicating that a reduction in MIR122 in the liver is associated with a systemic effect, whereas the administration of Compound 1 significantly reduced mice weight. The liver weight of the mice was in accordance with their body weight. The liver was further analyzed to assess lipotoxicity, and hepatic lipid droplets and TG content were reduced in Compound 1-treated mice, and this reduction was completely abolished in antagoMIR122 injected mice, suggesting that the beneficial effect of Compound 1 on steatosis is mediated by MIR122 activity.

We also measured a surrogate marker for energy expenditure, β-hydroxybutyrate, and found a decrease in energy expenditure upon reduction of MIR122 levels and an increase upon treatment with Compound 1. These effects were associated with reduced mature MIR122 levels in the liver, plasma and muscle tissues upon administration of antagoMIR122, and an increase of MIR122 when Compound 1 was administered, as seen in plasma and muscle.

Pri-MIR122 was not detected in muscle tissue, indicating that mature MIR122 is not expressed from the endogenous MIR122 promotor. The effect on muscle MIR122 levels was very similar to that seen in the plasma and was probably through MIR122 secretion effects. The level of mature MIR122 in the liver showed no increase following Compound 1 administration, probably due to its secretion to the plasma since the MIR122 precursor RNAs, pri- and pre-MIR122, increased significantly in the liver following Compound 1 administration.

Importantly, the MIR122 target gene Dgat1 was reduced in the liver following Compound 1 treatment whereas the RORα target gene FGF21 increased (FIG. 12).

The MIR122 target genes in the muscle, AldoA and Agpat1, were also affected in a respective manner. Furthermore, in the presence of Compound 1, the muscle TG levels were reduced. The level of the liver FGF21 mRNA correlated with pri-MIR122 levels, following Compound 1 administration, suggesting a co-regulation (FIG. 13). These observations support our hypothesis that controlling MIR122 levels, either by reducing its hepatic levels by antagoMIR122, or increasing it by Compound 1, has both, can stimulate endogenous production of FGF21. Thus, Compound 1 and other RORα agonists can be used to treat disorders, such as pancreatitis, which are associated with FGF21 levels.

### Compound 1 Reduces Steatosis Via Increased MIR122 Expression in HFD-Fed Mice.

Since Compound 1 is a potent MIR122 activator which exhibits beneficial biochemical effects, its effect on lipotoxicity and metabolism was determined. Compound 1 was administered to mice with an established NAFLD (following HFD feeding) which resulted in increased liver and plasma mature MIR122 levels, as well as increased MIR122 precursors and RORα targets in the liver (FIG. 14), similar to its effect on normal diet-fed mice.

The Anti-Inflammatory and Anti-Fibrogenic Effects of Activating the RORα-MIR122-Triglycerides Circuitry with Compound 1

Following our findings that the RORα activator Compound 1 displays significant metabolic benefits, we wanted to investigate its effects on liver inflammation and fibrosis. Towards this aim, we used the mouse atherogenic diet model. Following 3 weeks on the atherogenic diet, when liver inflammation and fibrosis have already developed, we initiated treatment with Compound 1 for additional 3.5 weeks, with 3 injections per week. Animals were then assessed for the effect of Compound 1 on a large number of processes. Compound 1 improved liver enzymes, reducing significantly AST and ALT levels. Mature MIR122 levels increased in the liver and in the plasma following the administration of Compound 1. H&E, CD3 and F4/80 staining demonstrate that Compound 1 improved liver inflammation significantly. This improvement in inflammation was associated with a significant reduction in liver fibrosis, as assessed by two measures, Masson Trichrome (M.T.) and smooth muscle actin (αSMA) staining. NK cells are known to target activated liver stellate cell. The resolution of fibrosis was associated with a significant reduction in NK cells in the livers of Compound 1-treated mice.

The effect of the RORα activator, Compound 1 was apparent also on fibrosis driver genes as seen in FIG. 12. These results strongly support the concept that RORα agonists reduce hepatic inflammation and fibrosis likely through MIR122 up-regulation. While not wishing to be bound to a particular theory, as there is a high degree of fibrosis in patients with pancreatitis, it is believed that RORα agonists can treat pancreatitis by reducing and/or reversing fibrosis.

### Discussion

In this report, we show that activating RORα offers significant health benefits. This RORα activity is mediated through induction of hepatic MIR122 levels, although additional RORα activities could potentially contribute to these beneficial effects. MIR122 in turn, increases expression of FGF21. RORα activation results also in increased secretion of hepatic MIR122 to the plasma, leading to increase in its level in remote tissues such as WAT, muscle and heart muscle, where MIR122 also affects its target genes. Hence, activation of RORα has both, a liver and a systemic effect.

We identified the synthetic RORα activator, Compound 1, among a number of compounds, by its strong enhancement of MIR122 promoter activity. By testing the effect of Compound 1 in several mice models, we found this compound to have a significant beneficial effect on increasing FGF21 levels, and thus can be used to treat pancreatitis.

Therapy for pancreatitis has a high priority since there is not a single approved drug today for pancreatitis, other than for palliative care. Therefore, pancreatitis patients are still without a therapeutic option. Many compounds are in the drug development pipeline, some showing interesting promises, and some failing to meet important endpoints. We decided to investigate the potential therapeutic effects of MIR122 due to its hormone-like features. Namely, MIR122, like many other microRNAs, is produced in one tissue, the liver, and is then secreted to the blood, from where it reaches remote tissues. We have shown that MIR122 exerts its anti-lipemic effect in the liver as well as in the remote tissues.

Hepatocytes produce large amounts of MIR122, reaching 250,000 copies per cell. The effective remote activity of MIR122, correlates with its high production and secretion to generate high plasma levels. Therefore, inducing a high production rate of MIR122 could be translated into an effective therapeutic compound. However, rather than developing a system in which a synthetic MIR122 (mimic-MIR122) is synthesized, made as a drug and injected to patients with NASH for years, we preferred to develop a small drug that induces the endogenous expression of the hepatic MIR122, and, subsequently, FGF21, and could be given to patients for a prolonged period.

In this report, we demonstrate that Compound 1 specifically activates RORα, which in turn, increases MIR122 levels in the liver, and leads to increased FGF21 expression. Based on our results, we propose that RORα activators are promising compounds to be developed and assessed for their clinical beneficial effects on pancreatitis in patients.

### Example 13

RORα Activation could Reverse Pancreatitis Through FGF21 Activation

Pancreatitis is a common and debilitating clinical condition that causes substantial morbidity and mortality. There are no specific therapies for this severe clinical condition. Treatments for pancreatitis are very limited and generally supportive only. Pancreatitis initiates from the activation of digestive enzymes in the pancreas, which causes tissue damage and inflammation. Common causes of pancreatitis include alcohol abuse, hyperlipidemia and gallstones movement out of the biliary system. Pancreatitis is also iatrogenic, occurs in 5 to 10% of patients undergoing endoscopic retrograde cholangiopancreatography (ERCP). Collectively, pancreatitis is an unmet therapeutic need.

Fibroblast growth factor 21 (FGF21) is a hormone secreted by the liver in response to diverse metabolic stresses. FGF21 is expressed in the exocrine pancreas, to stimulate digestive enzyme secretion. FGF21 KO mice are particularly susceptible to pancreatitis. Overexpression of FGF21 confers protection from pancreatitis. Prophylactic FGF21 administration reduces fibrogenesis in a mouse model of pancreatitis. Loss of FGF21 is a driving factor of pancreatitis. Using FGF21 therapeutically reverses preexisting pancreatitis.

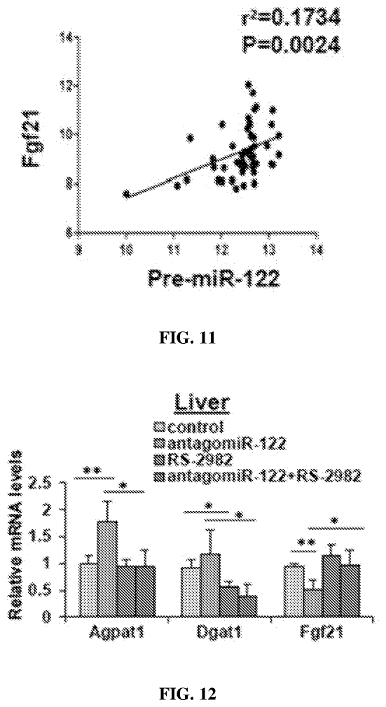

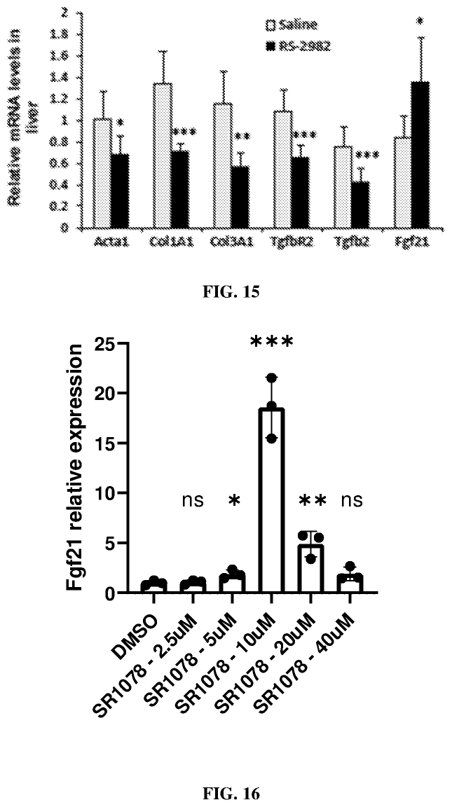

We have shown that FGF21 could be activated upon treating cells with RORα agonist Compound 1, as shown below directly and indirectly (see FIGS. 11-15)). Thus, our compounds that activate RORα have a unique anti-pancreatitis therapeutic potential.

### Example 15

### Pancreatitis Screening Assays

In order to evaluate the RORA agonist compounds described herein for their ability to treat, prevent, reduce the susceptibility to, reduce the severity of, or delay the progression of pancreatitis, it will be useful to employ an animal model of pancreatitis.

Though chronic alcohol abuse is the leading cause of chronic pancreatitis, a number of other etiologies, including toxins, obstructive lesions, and genetic disorders can cause chronic pancreatitis.

In the traditional model of acute pancreatitis, repeated bouts of acute pancreatitis lead to fibrosis. An example of this model is the induction of chronic pancreatitis through repetitive cerulein injections. The Sentinal Acute Pancreatitis Event (SAPE) model proposes that an initiating event, like an episode of acute pancreatitis, activates the immune system allowing risk factors to drive a pro-fibrotic, anti-inflammatory pathways that lead to chronic pancreatitis. Ethanol sensitizations models, such as the ethanol/LPS model conform to this hypothesis. Both models of pancreatitis can result in similar severity of final pancreatic injury.

All animal models of chronic pancreatitis, except autoimmune models, share the same histologic endpoints (i.e., fibrosis, pancreatic duct abnormalities, and cellular changes), whether caused by chemical exposure, dietary changes, infectious agents, genetic modifications, or mechanical obstructions. Examples of animals that can be used in these models include cats, dogs, ferrets, mice, rats, pigs, rabbits, and zebrafish.

These include some or all of the following features: chronic inflammation, stellate cell proliferation/activation, acinar cell dropout, ductal dilatation, intraductal calcifications, and nerve enlargement.

One animal model involves repeated intraperitoneal injections of the cholecystokinin (CCK) analogue, cerulein, which can be combined with lipopolysaccharide (LPS), chronic ethanol, cyclosporine A, or dibutyl tin dichloride (DBTC). Another animal model involves chronic administration of ethanol and LPS. Yet another model involves administration of relatively large doses of arginine. Still another involves administering a choline-deficient, ethionine-supplemented (CDE) diet. Another involves administration of tri-nitrobenzene sulfonic acid (TNBS).

Of these, repetitive administration of cerulein, sometimes in the presence of sensitizing agents, is the most commonly used model of chronic pancreatitis. These and other toxic compounds can be administered systemically, intraperitoneally, or by retrograde infusion into the pancreatic duct.

Treatment with supraphysiologic doses of cerulein, is employed in a widely used animal model of acute pancreatitis. At low doses, cerulein provides physiologic stimulation to CCK receptors and enhances secretion from the acinar cell. However, animals treated with high dose cerulein (10-100× physiologic doses; 20-50 g/kg, intravenously or intraperitoneally) develop mild to moderate acute interstitial pancreatitis. The cerulein model of acute pancreatitis is characterized by aberrant zymogen activation in the acinar cell, inhibition of secretion, increased inflammation, and cellular damage. However, in this model of pancreatitis, there is recovery of exocrine pancreatic structure and function within 24 to 48 hours.

The cerulein model of chronic pancreatitis requires repeated cerulein injections over time and is the most commonly used, reproducible model of chronic pancreatitis. There are a number of protocols that vary in dose, interval, and duration of cerulein injections (Feng et al., Int J Biol Sci 8: 249-257, 2012; Neuschwander-Tetri et al., Dig Dis Sci 45: 665-674, 2000; Yadav et al., Am J Gastroenterol 106: 2192-2199, 2011), though any of these protocols can be used. The cerulein model produces morphohistologic findings compatible with chronic pancreatitis in humans, including fibrosis, chronic inflammation, atrophy, transdifferentiation of acini into duct-like cells, and ductal dilatation.

The cerulein model of chronic pancreatitis serves as the basis for studying the sensitizing effects of other agents. Lipopolysaccharide (LPS), a bacterial endotoxin, is a particularly relevant agent because chronic alcohol consumption leads to increased gut permeability, predisposing to bacterial translocation and increased serum LPS levels. LPS has been shown activate pancreatic stellate cells and stimulate inflammatory cytokines through activation of toll-like receptor 4 (TLR4) and nuclear factor κB (NFκB). The addition of LPS to the repeated cerulein injection model accelerates the progression of disease and worsens its severity, measured by acinar cell atrophy, fibrosis, and the development of tubular complexes (Ohashi et al., Am J Physiol Gastrointest Liver Physiol 290: G772-G781, 2006).

Cyclosporine A (CsA) has also been used a sensitizing agent in cerulein-induced chronic pancreatitis. In this model, rats received only two doses of intraperitoneal cerulein during a 15-day treatment with intraperitoneal CsA. Rats treated with cerulein alone recover fully from the acute cerulein pancreatitis, while those co-treated with cyclosporine exhibit chronic pancreatitis with atrophy, mononuclear inflammatory infiltrate, and enhanced collagen deposition (Vaquero et al., Gut 45: 269-277, 1999).

Feeding of a choline deficient ethionine-supplemented (CDE) diet induces acute hemorrhagic pancreatitis in mice (Gilliland L and M. L. Steer, Am J Physiol 239: G418-G426, 1980). The mechanism responsible for CDE-induced pancreatic damage is not known. Long-term administration of the CDE diet intermittently over 24 weeks leads to histological changes consistent with chronic pancreatitis including acinar atrophy, fibrosis, and the development of tubular complexes. Additionally, increased expression of EGFR, SPINK3, and TGF-α, which are all implicated in the pathogenesis from chronic pancreatitis to pancreatic adenocarcinoma were observed in this model. However, even after 54 weeks of CDE feeding, malignant lesions did not form.

L-arginine, an essential amino acid, administered intraperitoneally in high doses, has been shown to cause severe, necrotizing acute pancreatitis in animal models (Mizunuma et al., J Nutr 114: 467-471, 1984). Repeated injections of lower doses of 1-arginine than cause severe acute disease over several weeks produce necrosis followed by chronic inflammation and fibrosis with impaired glucose tolerance in rats.

Intravenous or intraperitoneal injection of dibutyltin dichloride (DBTC), a compound used in the production of polyvinyl chlorides, leads to acute interstitial pancreatitis through direct toxicity on the acinar cell and by causing chronic biliary obstruction through the formation of obstructing plugs in the distal common bile duct (Merkord J and Hennighausen G, Exp Pathol 36: 59-62, 1989). When repeated DBTC injections are administered, rats develop chronic inflammation and fibrosis. However, this model is not highly reproducible, as only one third of animals display histological changes consistent with chronic pancreatitis.

### Retrograde Infusion of Toxic Substances

Several models involving the retrograde infusion of toxic substances have been attempted. These models deliver toxins only to the pancreas, unlike the models that require systemic toxin administration described above. Infusion of trinitrobenzene sulfonic acid into the pancreatic duct leads to acute necrotizing pancreatitis at 48 hours and fibrosis, inflammation, and atrophy consistent with chronic pancreatitis at later time points (Puig-Divi, et al., Pancreas 13: 417-424, 1996). Retrograde infusion of bile acids provides an attractive model to study acute pancreatitis because gallstone obstruction is a common cause of acute pancreatitis (Perides, et al., Gastroenterology 138: 715-725, 2010). This method is thought to elicit pancreatitis through direct toxic effects on the acinar cell that is mediated by the bile acid receptor Gpbar1.

Any of these models can be used to judge the effectiveness of the compounds described herein, alone or in combination with other active agents, in treating, preventing, reducing the susceptibility to, reducing the severity of, or delaying the progression of pancreatitis.

For example, one or more control animals can be administered cerulein according to one of the protocols discussed above, and one or more test animals administered cerulein while also being treated with an RORA agonist compound described herein. The progression of pancreatitis can be monitored in test and control animals. The prevention of pancreatitis, or reduced susceptibility, reduced severity, or delayed progression can thus be monitored.

Alternatively, one can wait until those animals administered the compounds which cause pancreatitis to develop actually develop pancreatitis, and then evaluate the effectiveness of the RORA agonist compounds described herein in treating the pancreatitis, reducing its severity, or delaying its progression.

### Combinations of Pharmaceutical Agents and RORA Agonists

There are (at least) seven classes of medications associated with acute pancreatitis: statins, ACE inhibitors, oral contraceptives/hormone replacement therapy (HRT), diuretics, antiretroviral therapy, valproic acid, and oral hypoglycemic agents. While the mechanisms by which these drugs cause pancreatitis are not known exactly, it is believed that statins have direct toxic effect on the pancreas or through the long-term accumulation of toxic metabolites. Meanwhile, ACE inhibitors cause angioedema of the pancreas through the accumulation of bradykinin. Birth control pills and HRT cause arterial thrombosis of the pancreas through the accumulation of fat (hypertriglyceridemia). Diuretics such as furosemide have a direct toxic effect on the pancreas. Meanwhile, thiazide diuretics cause hypertriglyceridemia and hypercalcemia, where the latter is the risk factor for pancreatic stones.

HIV infection itself can cause a person to be more likely to get pancreatitis, and antiretroviral drugs may cause metabolic disturbances, such as hyperglycemia and hypercholesterolemia, which predisposes to pancreatitis.

Valproic acid may have direct toxic effect on the pancreas. There are various oral hypoglycemic agents, such as metformin, that contribute to pancreatitis. Atypical antipsychotics such as clozapine, risperidone, and olanzapine can also cause pancreatitis.

Any of models discussed above can be used to judge the effectiveness of the compounds described herein, when combined with one of the active agents discussed above that can cause pancreatitis, in treating, preventing, reducing the susceptibility to, reducing the severity of, or delaying the progression of pancreatitis caused by these other active agents.

For example, one or more control animals can be administered a statin, ACE inhibitor, oral contraceptive/hormone replacement therapy (HRT), diuretic, antiretroviral therapy, valproic acid, or oral hypoglycemic agent such as metformin, optionally at doses higher than normal doses so as to accelerate the progression of pancreatitis, and treatment animals can be co-administered this active agent in combination with an RORA agonist, to determine the effectiveness of the RORA agonist in preventing, reducing the susceptibility to, reducing the severity of, or delaying the progression of pancreatitis caused by these other active agents. The prevention of pancreatitis, or reduced susceptibility, reduced severity, or delayed progression can thus be monitored.

Pharmaceutical compositions including an RORA agonist and a compound selected from the group consisting of statins, ACE inhibitors, oral contraceptives/hormone replacement therapy (HRT), diuretics, antiretroviral therapy, valproic acid, and oral hypoglycemic agents such as metformin, are within the scope of the embodiments described herein.

### Example 16

### Animal Models for Stroke

Animal models of stroke can be used to evaluate the effectiveness of the RORA compounds described herein in treating, preventing, reducing the susceptibility to, reducing the severity of, or delaying the progression of a stroke.

Animal models for stroke are well-known, and have been used to test recanalyzing, neuroprotective, neuroregenerative or anti-inflammatory drugs in pre-clinical setting.

One such animal model involves the distal occlusion of the middle cerebral artery (MCA) in rats. Different techniques and methods to induce focal and global ischemia of the brains have also been developed. Specific models mimic different types of stroke, focal and global ischemia.

Models of cerebral ischemia can be separated into focal and global ischemia models. Focal ischemia is characterized by a reduction of cerebral blood flow in a distinct region of the brain, whereas in global ischemia the reduction of blood flow affects the entire brain or forebrain (Traystman R J. Animal models of focal and global cerebral ischemia. ILAR journal/National Research Council, Institute of Laboratory Animal Resources. 2003; 44(2):85-95). In focal cerebral ischemia, either an artery or vein is occluded mechanically or by cerebral thromboembolism.

Stroke caused by an acute cerebral vessel occlusion can be reproduced by different techniques, namely by mechanical occlusion of either the proximal middle cerebral artery (pMCAo) (large vessel occlusion) or distal MCA (dMCAo) (small vessel occlusion), or by thrombotic occlusion either via injection of blood clots or thrombin into the MCA or by photo-thrombosis after intravenous injection of Rose Bengal.

pMCAo models are frequently used in stroke research. pMCAo is usually induced by direct mechanical occlusion, most often through the insertion of a silicon-coated nylon suture into the internal carotid artery that is subsequently advanced to the circle of Willis to occlude the MCA at its origin. The severity of ischemic injury can be modeled by leaving the suture filament in place either transiently for a variable duration of time (time usually ranges between 30-120 min) before the suture is removed to allow tissue reperfusion. In case of permanent pMCAo the suture is left in place and no reperfusion is allowed. Short-lasting pMCAo causes selective neuronal death in the lesion-sided striatum, expression of heat shock proteins, immediate early gene expression and induction of apoptotic signal pathways in the overlying cortex. Longer durations of occlusion instead result in brain infarcts that involve both the striatum and cortex, and may be associated with some animal mortality in case of edema formation.

Stroke in humans are most frequently caused by cerebral thromboembolism. Accordingly, a number of animal models has been developed that closely mimic the embolic occlusion of brain vessels. Embolic strokes can be induced in animals by injecting large-sized synthetic macrospheres (300-400 μm diameter) or small-sized microspheres (less than 50 μm) into the internal carotid artery. In the first case, large infarcts similar to those produced by the permanent occlusion of the MCA are induced. In the latter case, smaller, multifocal infarcts can occur (Gerriets, et al. J Neurosci Methods. 2003; 122(2):201-11; Miyake et al., Stroke. 1993; 24(3):415-20).

These models can be used to evaluate the effectiveness of the RORA agonists described herein in treating, preventing, reducing the susceptibility to, reducing the severity of, or delaying the progression of a stroke. To test treatment, or delaying the progression of a stroke, the compounds can be administered after the stroke has been mechanically induced. To test prevention, reducing the susceptibility to, or reducing the severity of stroke, the compounds can be administered before the stroke has been mechanically induced. When a combination of treatment and control animals are used, the effectiveness of the compounds can be compared with the control.

A different type of model is used to study thrombolytic therapies. Vascular occlusion is induced using autologous blood clots that are injected directly into the internal carotid artery (Kilic et al., Neuroreport. 1998; 9(13):2967-70). This type of animal model can be used to evaluate combination therapy using an RORA agonist compound described herein, in combination with an agent, like tPA, that dissolves the blood clots. Compositions including an RORA agonist compound and a compound that dissolves or otherwise removes blood clots are another embodiment of the invention described herein.

### Example 17

### Animal Model for Sarcopenia

Approximately 40-50% of the population over 80 years of age suffers from sarcopenia, making this condition a major geriatric clinical disorder and a key challenge to healthy aging. The hallmark symptom of sarcopenia is the loss of muscle mass and strength, and sarcopenic patients are likely to have worse clinical outcomes and higher mortality compared to healthy individuals.

Animal models designed to study sarcopenia include hind-limb unloading, de-nervation, and immobilization by using casts or wire strategies, as well as using aged rodents.

Aged rodents are commonly used in animal models for sarcopenia. For example, female C57BL/6J mice develop sarcopenia with significant loss of quadriceps muscle mass by 24 months, which is more pronounced by 27 to 29 months, at a time when there is denervation and altered neuromuscular junctions (NMJ) morphology of myofibers (Shavlakadze T and Grounds M, Bioessays. 2006; 28:994-1009; Chai et al., PLoS One. 2011; 6:e28090). Gait characteristics are also changed in aged mice. Compared to young mice (3 months old), aged mice (24 months old) exhibited significantly decreased cadence, increased stride-time variability, and altered footfall patterns. The aged-rat model also shows patterns of muscle decrease similar to those of an aged-mouse model.

Because high calorie intake is known to accelerate the setup of sarcopenia, some animal studies can involve providing animals with a high-fat diet.

Other animal models involve inducing muscle atrophy, such as by using hind-limb immobilization methods. These include head-down suspension with single hindlimb support, tail traction with tape, whole-body suspension with hindlimb-load bearing, and hindlimb tail-cast suspension. These “unweighting” models induce muscle loss.

Denervation is a common phenomenon in an aged neuromuscular junctions (NMJs). Some commonly-used rodent models use tibial- or sciatic-nerve transection to induce denervation. The tibial nerve is a mixed motor-sensory peripheral nerve in the rodent hindlimb and is 1 of the 3-terminal branches of the sciatic nerve. Transection of the tibial nerve denervates the gastrocnemius, soleus, and plantaris muscles.

If hindlimb functional assessment is desired, walking-track analysis can be performed at various time intervals. This involves dipping the animals' feet in ink, and allowing the animals to walk through an enclosure with paper on the bottom. Characteristics of the prints can be reliably measured and scored to indicate the extent of neuromuscular disability and gait compromise, since footprint characteristics reflect the functional muscle groups.

These animal models can be used to evaluate the ability of the RORA agonist compounds described herein in treating, preventing, reducing the susceptibility to, reducing the severity of, or delaying the progression of sarcopenia, particularly when used in conjunction with control animals that do not receive any treatment.

### Example 18

### Animal Model for Traumatic Brain Injury

Animal models of traumatic brain injury (TBI) are used to identify potential neuroprotective therapies for developing and adult brains.

Traumatic brain injury is a complex process and consists of four overlapping phases, which include primary injury, evolution of the primary injury, secondary or additional injury, and regeneration. Primary injury to the brain can be induced by numerous mechanisms. One mechanism involves direct contusion of the brain from the skull. Another mechanism involves brain contusion caused by a movement against rough interior surfaces of the skull, and/or indirect (contracoup) contusion of the brain opposite the side of the impact. Another mechanism involves shearing and stretching of the brain tissue caused by motion of the brain structures relative to the skull and each other. Another mechanism involves vascular response to the impact including subdural hematoma produced by rupture of bridging blood vessels located between brain and dura mater, decreased blood flow due to increased intracranial pressure or infarction and brain edema caused by increased permeability of cerebral blood vessels.

Diffuse axonal injury has been recognized as one of the main consequences of blunt head trauma; it is characterized by morphological and functional damages of axons throughout the brain and brainstem and leads to diffuse degeneration of cerebral white matter. Secondary injury mechanisms include complex biochemical and physiological processes, which are initiated by the primary insult and manifest over a period of hours to days.

Animal models seek to replicate certain pathological components or phases of clinical trauma in experimental animals, which then allows one to evaluate putative treatments. Rodent models are typically used for neurotrauma research. Their relatively small size permits repetitive measurements of morphological, biochemical, cellular, and behavioral parameters that require relatively large numbers of animals. The animals are typically subjected to one of the two major categories of experimental brain injury, namely, acceleration concussion and percussion concussion.

Mechanical force inflicts either dynamic or static brain trauma, depending on its amplitude, duration, velocity and acceleration. The mechanical force in static models possesses defined amplitude and duration, but no velocity or acceleration. Inherently, the static models usually focus on morphological and functional processes involved in injury. One example of a static injury model involves crushing a cranial nerve with forceps for a defined period of time.

Dynamic brain injury can be induced by applying mechanical force, with well-characterized amplitude, duration, velocity, and/or acceleration. Dynamic brain trauma can be further subdivided into direct and indirect injury. In the case of indirect dynamic brain injury, the mechanical force is generally directed at the whole body with the kinetic energy of the oscillating pressure waves that traverse the body imparting their effects on brain tissue.

Penetrating head injury and other direct brain deformation models are caused by the impact energy, which is delivered to the brain parenchyma through a skull perforated by a missile or a craniotomy. The use of these models to evaluate pharmaceutical treatments for TBI is well-established (Faden et al., Science, 1989 May 19; 244 (4906):798-800).

The lateral fluid percussion model provides an injury that replicates clinical contusion without skull fracture, and shows a direct relationship between the majority of pathological alterations and injury severity. It is widely used in neurotrauma research for both mechanistic studies and for drug screening.

Other models use controlled cortical impact to cause traumatic brain injury in rats (Dixon et al., J Neurosci Methods. 1991 October; 39(3):253-62).

These and other animal models can be used to evaluate the effectiveness of the RORA agonist compounds described herein in treating TBI, lessening its severity or duration, or reducing its progression, particularly when comparisons can be made between treatment and control animals. The compounds can be administered prior to, concomitantly with, or following the induction of brain injury, optionally in combination with other active agents used to treat traumatic brain injury. Pharmaceutical compositions comprising the RORA agonist compounds and the additional active agents are within the scope of the inventions described herein.

### Example 19

Pancreatitis is an example of inflammation of the pancreas. The most common causes of pancreatitis include gallstones (40%), alcohol abuse (33%), idiopathic (15-25%) and post endoscopic retrograde cholangiopancreatography (ERCP) (5-10%). Treatments for pancreatitis are limited, and are generally supportive in nature. The overall mortality rate in acute pancreatitis is 10-15%. Thus, there is an urgent need to find a treatment for pancreatitis.

According to some recent papers, FGF21 can be a treatment for pancreatitis (Hernandez, G. et al. Pancreatitis is an FGF21-deficient state that is corrected by replacement therapy. Science Translational Medicine 12, (2020)). ROR-α is one of the transcription factors that regulates FGF21 (Luo, Y. et al. Oncogenic KRAS Reduces Expression of FGF21 in Acinar Cells to Promote Pancreatic Tumorigenesis in Mice on a High-Fat Diet. Gastroenterology 157, 1413-1428.e11 (2019)).

ROR-α agonists such as RS2982 and the compounds described herein can be evaluated as a treatment for pancreatitis using models where pancreatitis is induced, for example, the two different mice models described herein.

In the first model, caerulein induced pancreatitis (CIP) (see, for example, Hyun, J. J. & Lee, H. S. Experimental models of pancreatitis. Clinical Endoscopy 47, 212-216 (2014)), 6-10 week old mice are injected seven hourly intraperitoneal injections of caerulein (50 ug/kg). The control group is injected with saline. 24 hours after the first injection, mice are injected with RS2982 (2.5-25 mg/kg) or DMSO and the mice pancreatitis is examined one day later.

In the second model, alcohol induced pancreatitis (AIP), 6-10 weeks old mice are injected intraperitoneal with ethanol (1.3 g/kg) and POA (150 mg/kg) twice over 1 hour (Huang, W. et al. Fatty acid ethyl ester synthase inhibition ameliorates ethanol-induced Ca2+-dependent mitochondrial dysfunction and acute pancreatitis. Gut 63, 1313-1324 (2014)). 24 hours after the first injection, mice are injected with RS2982 (2.5-25 mg/kg) or DMSO and the mice pancreatitis is examined one day later.

Using one of these models, RT-qPCR analysis of FFG21 mRNA expression was performed in 266-6 murine acinar cells after 6 hours treatment of DMSO/different doses of SR1078 (commercial ROR-α agonist). The statistical significance was calculated relative to DMSO and was determined by Student's t test (two-tailed). Values in graphs are mean±SD. *p<0.05, **p<0.01, ***p<0.001, ****p<0.0001.

The data, shown in FIG. 16, shows that SR1078 increases Fgf21 expression in acinar cells. Optimal effects were observed at a 10 micromolar (μM) concentration, with the effect decreasing as the dose was increased to 20 and then 40 micromolar (μM).

### Example 20: Assay for Identifying Potent RORα (RORA) Agonists

The ability of the compounds to bind the RORA receptor, to have agonist activity when bound to this receptor, and to not cross the blood brain barrier and/or not bind GABA receptors, are important considerations for compounds useful in the methods described herein.

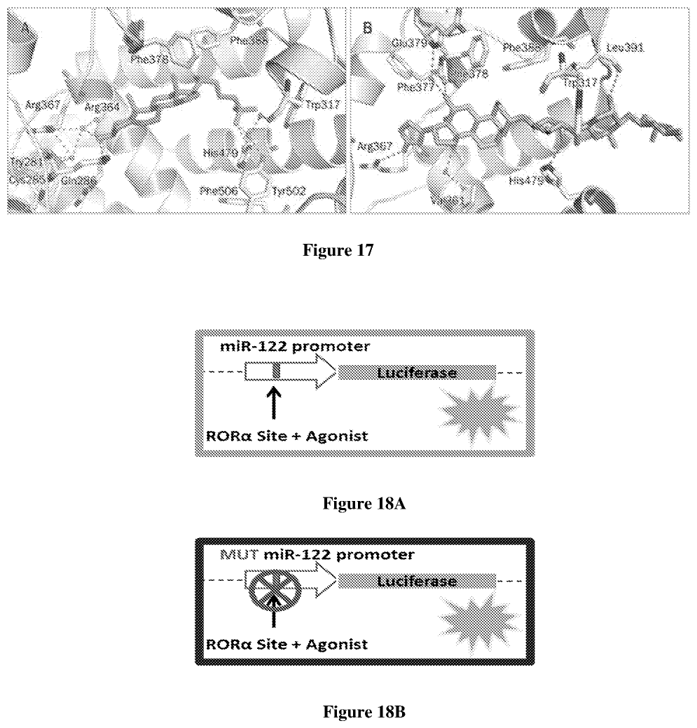

Agonists, inverse agonists, antagonists bind in same binding pocket of RORA LBD (ligand binding domain). The binding domain is shown in FIG. 17, along with compounds embedded within the domain.

There are a number of compounds known to be agonists and inverse agonists of the RORA receptor:

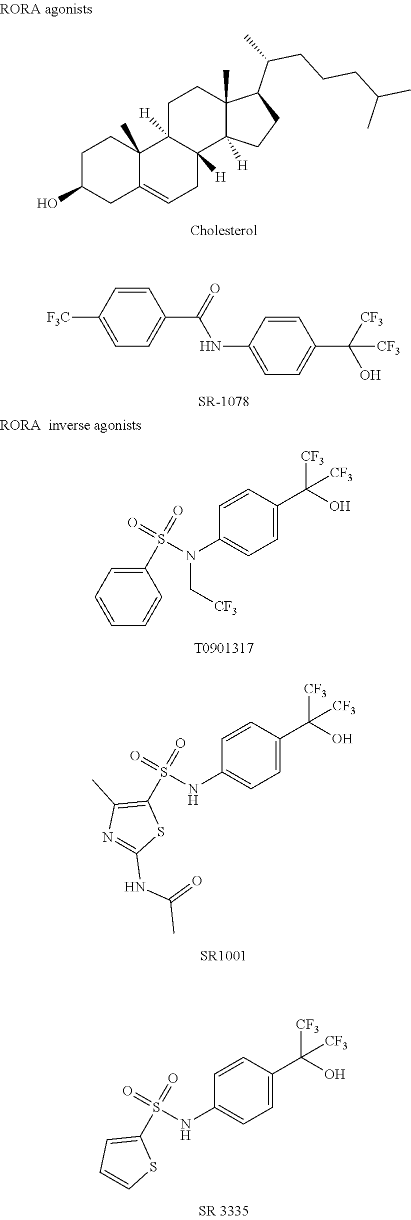

It is therefore important not only to identify compounds that bind to the RORA receptor with relatively high affinity, but also to identify compounds that are agonists, and not antagonists, inverse agonists, and the like.

RORα agonists can be used to increase levels of hepatic microRNA122 (MIR122) expression, which in turn can be used to treat pancreatitis, sarcopenia, stroke, glioblastoma, and traumatic brain injury, as well as fatty liver disease, including fatty liver (NASH) and related disorders. Increasing expression of RORA in livers of mice increases expression of MIR122 and reduces lipotoxicity.

The assay disclosed in Chai et al. (Chai et al., “Agonist of RORA Attenuates Nonalcoholic Fatty Liver Progression in Mice via Up-regulation of MicroRNA 122,” Gastroenterology. 2020; 159(3):999-1014.e9), is one example of an assay that can be used to identify RORA agonists. This assay was used to screen a library of compounds and identify RORα agonists.

### Materials and Methods.

### Cell Culture

The human hepatocellular carcinoma cell line Huh7 was cultured in Dulbecco's modified Eagle medium supplemented with 10% fetal calf serum, 1% penicillin/streptomycin (Thermo Fisher Scientific, Waltham, MA). Cells were cultured at 37° C. in a humidified atmosphere containing 5% CO₂, except for an experiment in which cells were placed in 32° C., as indicated in the text.

### Plasmids

The human MIR122 promoter fragments spanning the region from −900 base pairs relative to the transcription start site and mutating the RORA binding site (plasmids pMIR122-900 and pMIR122-RORA mut, respectively) were generated as described previously.

### Transfections

For luciferase assays, cells grown in 24-well plates were co-transfected with a luciferase reporter plasmid (50 ng) and 1 ng of Renilla luciferase vector (PRL, Promega, Madison, WI) with Lipofectamine LTX (Invitrogen, Waltham, MA) transfection reagent. For all experiments, the transfection performed using serum-free medium (Opti-MEM; catalog no. 31985070; Thermo Fisher Scientific).

### Luciferase Activity Assay

After transfections, the cells were lysed with passive lysis buffer (catalog no. E1941, Promega), shaken for 20 minutes at room temperature, and transferred into the appropriate 96-well plate. Firefly and Renilla luciferase activity was assessed using the Dual Luciferase Reporter Assay system (catalog no. E1910, Promega) on a luminometer Mithras 2000 (Centro XZ, LB960; Berthold Technologies, Bad Wildbad, Germany). The luciferase activity was normalized to Renilla luciferase activity. Readings were taken in triplicate.

A targeted virtual screen can be screened to identify novel RORA agonists. A library of commercially available compounds can be docked and scored into a model of the crystal structure of the RORA ligand-binding domain complexed with cholesterol sulfate.

Once lead compounds are identified, they can be screened for activity testing, for example, by assaying their induction of the MIR122 promoter using the luciferase promoter reporter plasmid as discussed above. As shown in FIGS. 18 A and B, a compound that binds to the appropriate miR-122 promoter site, and, once bound, shows agonist activity, will induce lucerifase activity (FIG. 18 A), and thus be identified as an RORA agonist. If the compound does not bind to the appropriate site, and/or does not induce lucerifase activity (FIG. 18 B), then it will not be identified as an RORA agonist. This screening assay can therefore be used to identify potential RORA agonists.

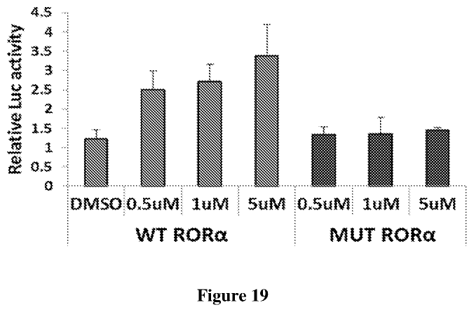

Using this screening assay, Compound 68 was identified as a potent MIR122 promoter, more potent than the commercial synthetic RORA agonist SR1078. The results of the screening assay, at concentrations of 0.5, 1 and 5 M, for both wild type and mutant RORA, are shown in FIG. 19.

Compound 68 has the following formula:

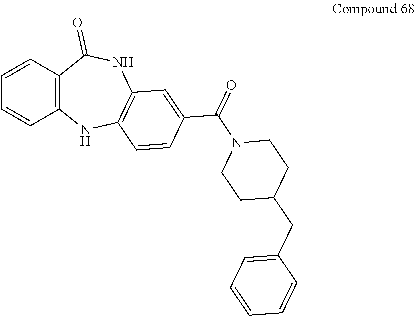 [SMILES: O=C1Nc2cc(C(=O)N3CCC(Cc4ccccc4)CC3)ccc2Nc2ccccc21]

Since substitution on one or more of the aryl rings, or variance in the size of the heterocyclic ring, would not be expected to significantly alter the affinity of the compounds for the RORA receptor, or alter the activity, or alter the ability of the compounds to cross the blood brain barrier and/or bind to the GABA receptors, a general formula for these compounds is shown below, and these compounds are within the scope of the compounds described herein:

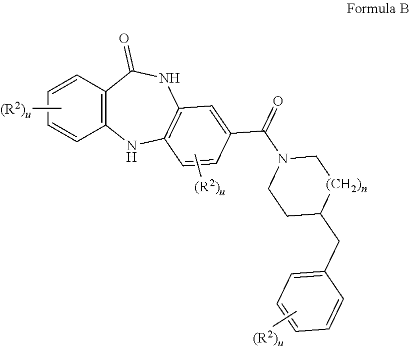

and pharmaceutically-acceptable salts and prodrugs thereof, wherein R² and u are as defined above with respect to Formula A, except that u can also be 0, and n is 0, 1 or 2.

Because Compound 68 has a benzodiazepine core:

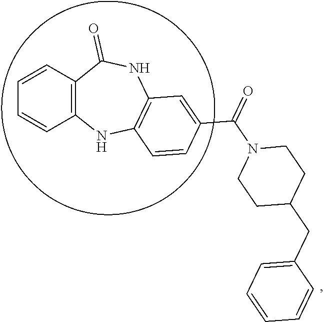 [SMILES: O=C1Nc2cc(C(=O)N3CCC(Cc4ccccc4)CC3)ccc2Nc2ccccc21]

other benzodiazepines also potentially have RORA activity and can be screened using the assay described above for this activity.

Representative benzodiazepines (BDZs) known to have pharmacological activity, though not known to bind to the RORA receptor, are shown below:

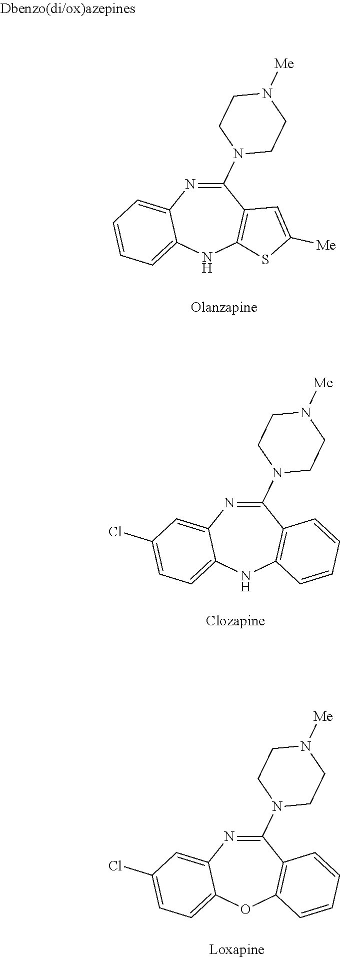 [SMILES: Cc1cc2c(s1)Nc1ccccc1N=C2N1CCN(C)CC1 ; CN1CCN(C2=Nc3cc(Cl)ccc3Nc3ccccc32)CC1 ; CN1CCN(C2=Nc3cc(Cl)ccc3Oc3ccccc32)CC1]

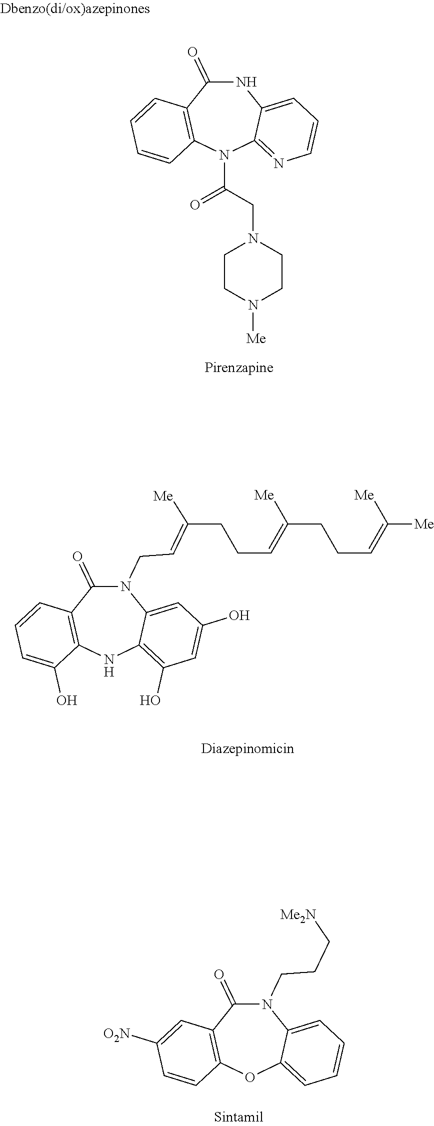 [SMILES: CN1CCN(CC(=O)N2c3ccccc3C(=O)Nc3cccnc32)CC1 ; CC(C)=CCC/C(C)=C/CC/C(C)=C/CN1C(=O)c2cccc(O)c2Nc2c(O)cc(O)cc21 ; CN(C)CCCN1C(=O)c2cc([N+](=O)[O-])ccc2Oc2ccccc21]

BDZ drugs are typically used to treat anxiety, seizures, and insomnia, and to provide sedation, and their mode of activity in the central nervous system is conventionally associated with their binding to GABA receptors, such as the GABA-A receptor, in the brain. In order to bind with GABA-A receptor, the drugs have to pass through blood brain barrier (BBB).

In certain embodiments, the compounds cross the blood brain barrier and also exhibit RORA agonist activity, and in these embodiments, they can be used to treat traumatic brain injury. In these embodiments, the effects of the compounds on the GABA-A receptor must also be considered, but where it is acceptable or desirable to take advantage of these effects while also treating a patient with traumatic brain injury, the compounds can be effective in such treatment.

In other embodiments, the compounds do not cross the blood brain barrier, or do not do so in appreciable concentrations, so have minimal or no effect on GABA receptors in the brain. As such, they will not have the effects traditionally associated with BDZ drugs, i.e., will not cause sedation, and can be used to treat the disorders discussed herein other than traumatic brain injury.

There are predictive models for determining whether a compound is CNS-active, such as the assay disclosed in Gourdeau, H., McAlpine, J. B., Ranger, M. et al. Identification, characterization and potent antitumor activity of ECO-4601, a novel peripheral benzodiazepine receptor ligand. Cancer Chemother Pharmacol 61, 911-921 (2008). Using this assay, the compound Diazepinomicin was predicted to be CNS inactive, and this prediction was later experimentally proven. Accordingly, this assay is a reasonable predictor of the CNS activity of the BDZ and other compounds described herein. Briefly, the assay involves screening the compounds for their ability to bind two different receptors, namely, the peripheral and the central benzodiazepine receptors. Where a compound binds the peripheral but not the central benzodiazepine receptor, or exhibits significantly higher binding affinity for the peripheral over the central benzodiazepine receptor (i.e., a ratio of 5/1 or more, 10/1 or more, 20/1 or more, or, most preferably, 50/1 or more, the compounds are not expected to exhibit significant CNS side effects, even if they cross the blood brain barrier. Assays for screening compounds for their binding affinity to various receptors, such as the peripheral and central benzodiazepine receptors, are well known to those of skill in the art, and need not be discussed in more detail here.

Using this assay, a series of BDZ compounds was screened, and the results are shown in the Table below.

| Drug Molecule | CNS | QPlogBB | QPlogS |
|---|---|---|---|
| Compound 68 | −1 | −0.991 | −6.808 |
| Olanzapine | 2 | 0.758 | −4.230 |
| Clozapine | 2 | 0.895 | −4.240 |
| Loxapine | 2 | 0.991 | −3.870 |
| Pirenzapine | 1 | 0.158 | −1.003 |
| Diazepinomicin | −2 | −2.097 | −7.326 |
| Sintamil | 0 | −0.854 | −1.456 |

In the table above, the column “CNS” shows predicted central nervous system activity on a −2 (inactive) to +2 (active) scale. The column “Q log BB” shows a predicted brain/blood partition coefficient. The ideal range of q Log BB for drugs to avoid BBB is −3.0-1.2. The more positive the number, the more likely the compound is to pass the BBB. The column Q log S shows a predicted aqueous solubility, log S, where S in mol/dm⁻³ is the concentration of solute in a saturated solution that is in equilibrium with the crystalline solid. An ideal range of Q log S for the compounds described herein is −6.5-0.5, where the more negative the value, the less soluble the compound is. Using these predictive models, it is believed that Compound 68 is unlikely to be CNS active.

These models can also be used to screen other benzodiazepine derivatives, including the compounds described herein.

### Bromodomain Inhibitors (BDZ Derivatives)

The compounds (+)-JQ1, (+)-MS417, and I-BET are benzodiazepine derivatives that are also known to be bromodomain inhibitors, and to be non-CNS active (Smith et al., “Privileged Diazepine Compounds and Their Emergence as Bromodomain Inhibitors,” Chemistry & Biology, Volume 21, Issue 5, Pages 573-583 (2014)). Their structures are shown below:

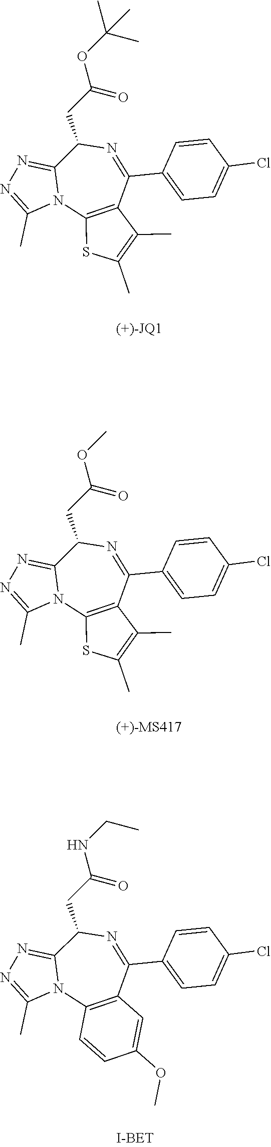 [SMILES: Cc1sc2c(c1C)C(c1ccc(Cl)cc1)=N[C@@H](CC(=O)OC(C)(C)C)c1nnc(C)n1-2 ; COC(=O)C[C@@H]1N=C(c2ccc(Cl)cc2)c2c(sc(C)c2C)-n2c(C)nnc21 ; CCNC(=O)C[C@@H]1N=C(c2ccc(Cl)cc2)c2cc(OC)ccc2-n2c(C)nnc21]

As was done with the compounds in the table above, these three compounds were also evaluated for their likelihood of crossing the blood brain barrier. The results are shown in the table below:

| Drug Molecule | CNS | QPlogBB | QPlogS |
|---|---|---|---|
| Compound 68 | −1 | −0.991 | −6.808 |
| Olanzapine | 2 | 0.758 | −4.230 |
| Clozapine | 2 | 0.895 | −4.240 |
| Loxapine | 2 | 0.991 | −3.870 |
| Pirenzapine | 1 | 0.158 | −1.003 |
| Diazepinomicin | −2 | −2.097 | −7.326 |
| Sintamil | 0 | −0.854 | −1.456 |
| (+)-JQ1 | 0 | −0.238 | −6.976 |
| (+)-MS417 | 0 | −0.394 | −5.985 |
| I-BET | 0 | −0.511 | −5.339 |

Based on this information, these and other bromodomain inhibitors are predicted to be less CNS active than other BDZ drugs in the list.

Conventional BDZ drugs show CNS activity by binding to GABA-A receptor. To further determine whether the compounds might have CNS activity if they did cross the blood brain barrier, a study can be performed to determine binding to the GABA-A receptor.

Molecular docking can be performed with pdb structures of GABA-A receptor, for example, using one or more protein databank structures of the GABA-A receptor, such as 6X3X (Human GABAA receptor alpha1-beta2-gamma2 subtype in complex with GABA plus diazeparm), 6X3U (Human GABAA receptor alpha1-beta2-gamma2 subtype in complex with GABA plus flumazenil)). A representative in silico molecular docking study was performed for COMPOUND 68 with two pdb structures of GABA-A receptor (protein databank (pdb) id 6X3X and 6X3U. Based on the docking score, COMPOUND 68 binds with GABA-A receptor with less affinity than known BDZ drugs, and thus would be expected to show little CNS activity even if it crossed the blood brain barrier.

Virtual screening has proven to be a very successful approach for finding ligand hits and assisting lead optimization in structure-based drug discovery projects. By docking a large library of compounds into one or more high-resolution structures of the target receptor, fewer compounds typically need to be experimentally screened to identify prospective lead optimization candidates.

Beyond identifying small molecules likely to bind well to a protein target, docking methods are used in a variety of context such as polypeptide and macrocycle pose prediction, predicting protein-ligand complex geometries, and preparing congeneric series for binding affinity prediction with methods such as Free Energy Perturbation or MM-GBSA. This approach involves averaging gas-phase energies (MM) and solvation free energies as determined by Generalized Born models (GB/SA) (see, for example, Gohlke and Case, Computational Chemistry, Volume 25, Issue 2, Pages 238-250 (2004)). Moving beyond the rigid receptor approximation common in structure-based virtual screening, the Induced Fit docking protocol predicts the effect of ligand docking on protein structure.

### Glide Docking and Scoring Methodology

The Glide HTVS, SP and XP docking methodologies are well known. Glide HTVS and SP use a series of hierarchical filters to search for possible locations of the ligand in the binding-site region of a receptor. The shape and properties of the receptor are represented on a grid by different sets of fields that provide progressively more accurate scoring of the ligand pose. Exhaustive enumeration of ligand torsions generates a collection of ligand conformations that are examined during the docking process. Given these ligand conformations, initial screens are deterministically performed over the entire phase space available to the ligand to locate promising ligand poses. From poses selected by initial screening, the ligand is refined in torsional space in the field of the receptor using OPLS34 (Glide SP & XP) or OPLS2005 (GLIDE HTVS) with a distance-dependent dielectric model. Finally, a small number of poses can be minimized within the field of the receptor with full ligand flexibility (post-docking minimization or PDM).

The molecular mechanics energies combined with the Poisson-Boltzmann or generalized Born and surface area continuum solvation (MM/PBSA and MM/GBSA) methods can be used for estimating the free energy of the binding of small ligands to biological macromolecules (Genheden S, Ryde U. The MM/PBSA and MM/GBSA methods to estimate ligand-binding affinities. Expert Opin Drug Discov. 2015; 10(5):449-461).

Lead compounds identified using these methods can be evaluated for their usefulness as potential therapeutics by evaluating, in an in vivo, in vitro or in silico manner, their adsorption, distribution, metabolism, and excretion (collectively, ADME) and toxicity. One representative method for an in silico assessment of the adsorption, distribution, metabolism, and excretion (collectively, ADME) is QikProp (Schrodinger). QikProp predicts a wide range of predicted properties, including octanol/water and water/gas log Ps, log S, log BB, overall CNS activity, Caco-2 and MDCK cell permeabilities, log Khsa for human serum albumin binding, and log IC₅₀ for HERG K+-channel blockage. This allows for determination of a molecule's suitability as a potential therapeutic agent. QikProp bases its predictions on the full 3D molecular structure, and, as such, can provide accurate results in predicting properties for molecules with novel scaffolds as for analogs of well-known drugs. QikProp rapidly screens compound libraries for useful hits, identifying molecules with computed properties that fall outside the normal range of known drugs, making it simple to filter out candidates with unsuitable ADME properties.

One representative method for an in silico assessment of toxicity is Derek Nexus. Derek Nexus predicts potential toxicity for most toxicological endpoints, including carcinogenicity, mutagenicity, genotoxicity, skin sensitization, teratogenicity, irritation, respiratory sensitization, and reproductive toxicity.

Accordingly, the in silico ability of the compounds to bind to various receptors can be evaluated using Docking—Glide SP/XP MM-GBSA (MM-PBSA can alternatively be used), docking with RORB/C & RORA inverse agonist structure. Using this approach, a library of benzodiazepine molecules was screened for potential CNS activity. The following lead compounds were identified.

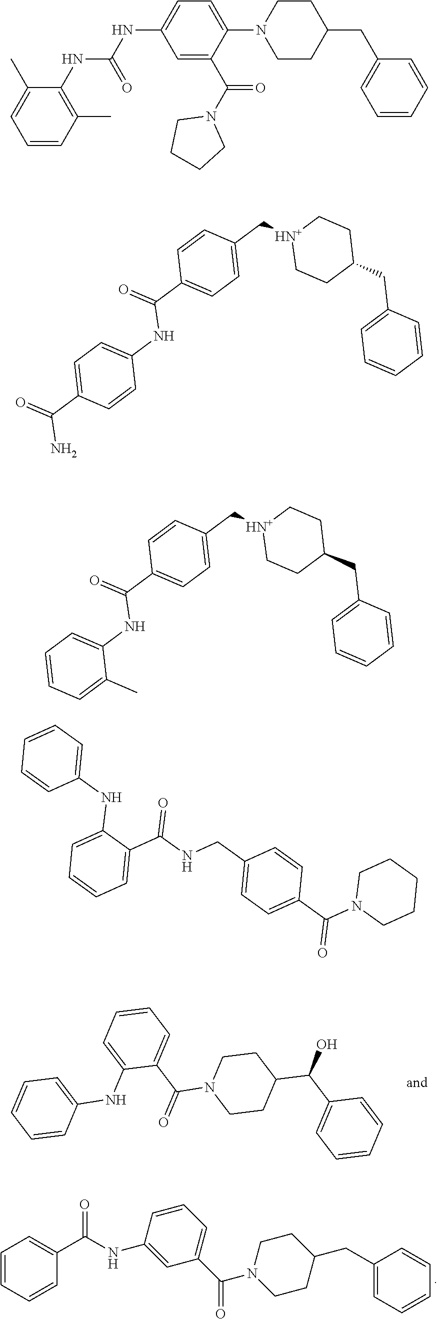 [SMILES: Cc1cccc(C)c1NC(=O)Nc1ccc(N2CCC(Cc3ccccc3)CC2)c(C(=O)N2CCCC2)c1 ; NC(=O)c1ccc(NC(=O)c2ccc(C[N@H+]3CC[C@H](Cc4ccccc4)CC3)cc2)cc1 ; Cc1ccccc1NC(=O)c1ccc(C[N@H+]2CC[C@@H](Cc3ccccc3)CC2)cc1 ; O=C(NCc1ccc(C(=O)N2CCCCC2)cc1)c1ccccc1Nc1ccccc1 ; O=C(c1ccccc1Nc1ccccc1)N1CCC([C@@H](O)c2ccccc2)CC1 ; O=C(Nc1cccc(C(=O)N2CCC(Cc3ccccc3)CC2)c1)c1ccccc1]

While not wishing to be bound to a particular theory, it is believed that substitution on the aromatic rings will not substantially alter the activity of the compounds, either as RORA agonists or GABA-A agonists, or their ability to cross the blood brain barrier. Accordingly, the following general formulas of compounds are expected to exhibit binding affinity to the RORA receptor, agonist activity once bound, and selectivity for RORA over GABA-A and/or inability or low ability to cross the blood brain barrier:

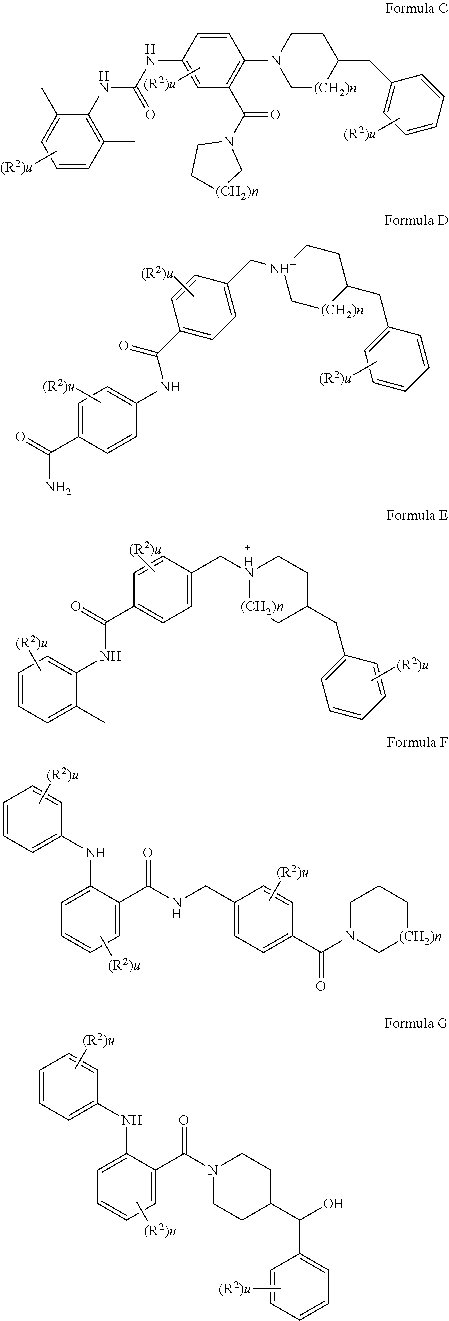

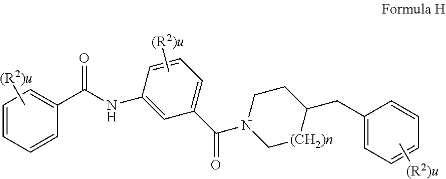

and pharmaceutically-acceptable salts and prodrugs thereof, wherein R² and u are as defined above with respect to Formula A, except that u can be 0, and n is 0, 1 or 2.

Based on the information obtained using the screening assays discussed in this example, it is believed that the compounds of Formulas B—H will be agonists of the RORA receptor, will bind with high affinity to the RORA receptor, will not bind with high affinity to GABA receptors, such as the GABA-A receptor, and will not cross the blood brain barrier. Using the screening assays described in this example, individual compounds can be tested to confirm these properties.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

1. A method for treating, preventing, reducing the susceptibility to, reducing the severity of, or delaying the progression of a disorder selected from the group consisting of pancreatitis, sarcopenia, stroke, glioblastoma, and traumatic brain injury, comprising administering to a patient in need thereof an effective amount of a compound of Formula (A): 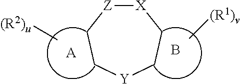
or a pharmaceutically acceptable salt or prodrug thereof, wherein:
wherein one of X and Z is selected from the group consisting of —NH—, —N(NH₂)—, —N(OH)—, —N(CH₂—O—P(O)(OH)₂)—; N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, —O—, —CH₂—, —CH(C₁₋₁₀ alkyl)-, C(C₁₋₁₀ alkyl)₂-, —CH(C₃₋₁₀ cycloalkyl)-, —CH(C₂₋₁₀ alkenyl, —CH(C₂₋₁₀ alkynyl)-, —CH(aryl)-, —CH(heteroaryl)-, —CF₂—, —CCl₂—, —CH(CF₃)—, —CH(OH)—, —CH(O—C₁₋₁₀ Alkyl)-, —CH(NH₂)—, —CH(NH—C₁₋₁₀ Alkyl)-, and —CH(C(O)NH₂)—,
and the other one of X and Z is selected from the group consisting of —C(O)—, —SO₂—, —N(C(O)—, —CH₂—, —CH(C₁₋₁₀ alkyl)-, C(C₁₋₁₀ alkyl)₂-, —CH(C₃₋₁₀ cycloalkyl)-, —CH(C₂₋₁₀ alkenyl, —CH(C₂₋₁₀ alkynyl)-, —CH(aryl)-, —CH(heteroaryl)-, —CF₂—, —CCl₂—, —CH(CF₃)—, —CH(OH)—, —CH(OAlkyl)-, —CH(NH₂)—, —CH(NHC₁₋₁₀ Alkyl)-, and —CH(C(O)NH₂)—,
Y is selected from the group consisting of —NH, —N(NH₂)—, —N(OH)—, —N(CH₂—O—P(O)(OH)₂)—; N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, —O—, —CH₂—, —CH(C₁₋₁₀ alkyl)-, —CH(C₃₋₁₀ cycloalkyl)-, —CH(C₂₋₁₀ alkenyl, —CH(C₂₋₁₀ alkynyl)-, —CH(aryl)-, —CH(heteroaryl)-, —C(C₁₋₁₀ alkyl)₂-, —CF₂—, —CCl₂—, —CH(CF₃)—, —CH(OH)—, —CH(O—C₁₋₁₀ Alkyl)-, —C(O)—, —SO₂—, —N(C(O)—C₁₋₁₀ Alkyl)-, —N(C(O)O—C₁₋₁₀ Alkyl)-, —CH(NH₂)—, —CH(NH—C₁₋₁₀ Alkyl)-, and —CH(C(O)NH₂)—,
A and B are, independently, phenyl, a five-membered heteroaromatic ring containing one, two or three nitrogen, oxygen, or sulfur atoms, or a six-membered heteroaromatic ring containing one, two or three nitrogen atoms;
u and v are independently 0, 1, 2, 3 or 4; with the proviso that at least one of u and v is 1, 2, 3, or 4;
each R¹ and R² are independently R³, OH, OR³, SR³, S(O)R³, SO₂R³, C(O)R³, C(O)OR³, OC(O)R³, OC(O)OR³, NH₂, NHR³, NHC(O)R³, NR³C(O)R³, NHS(O)₂R³, NR³S(O)₂R³, NHC(O)OR³, NR³C(O)OR³, NHC(O)NH₂, NHC(O)NHR³, NHC(O)N(R³)₂, NR³C(O)N(R³)₂, C(O)NH₂, C(O)NHR³, C(O)N(R³)₂, C(O)NHOH, C(O)NHOR³, C(O)NHSO₂R³, C(O)NR³SO₂R³, SO₂NH₂, SO₂NHR³, SO₂N(R³)₂, COOH, C(O)H, C(N)NH₂, C(N)NHR³, C(N)N(R³)₂, C(N)OH, C(N)OCH₃, CN, N₃, NO₂, CF₃, CF₂CF₃, OCF₃, OCF₂CF₃, halo (F, Cl, Br, or I), —CH₂-phosphonate, —CH₂O-phosphate, CH₂P(O)(OH)₂, CH₂P(O)(OR³)₂, CH₂P(O)(OR³)(NR³), CH₂P(O)(NR³)₂, CH₂P(O)(OH)(OC₁₋₁₀ alkyl-O—C₁₋₂₀ alkyl), or CH₂-cycloSal monophosphate prodrug,
wherein the term phosphate includes monophosphate, diphosphate, triphosphate, and stabilized phosphate prodrugs, and the term phosphonate includes the same prodrugs that are present in the phosphate prodrugs,
and when R¹ and R² are on adjacent carbon, they can come together to form an saturated or unsaturated alkyl, an aromatic or a heteroaromatic ring,
each R³ is, independently, aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁴, OH, OR⁴, SR⁴, S(O)R⁴, SO₂R⁴, C(O)R⁴, C(O)OR⁴, OC(O)R⁴, OC(O)OR⁴, NH₂, NHR⁴, NHC(O)R⁴, NR⁴C(O)R⁴, NHS(O)₂R⁴, NR⁴S(O)₂R⁴, NHC(O)OR⁴, NR⁴C(O)OR⁴, NHC(O)NH₂, NHC(O)NHR⁴, NHC(O)N(R⁴)₂, NR⁴C(O)N(R⁴)₂, C(O)NH₂, C(O)NHR⁴, C(O)N(R⁴)₂, C(O)NHOH, C(O)NHOR⁴, C(O)NHSO₂R⁴, C(O)NR⁴SO₂R⁴, SO₂NH₂, SO₂NHR⁴, SO₂N(R⁴)₂, COOH, C(O)H, C(N)NH₂, C(N)NHR⁴, C(N)N(R⁴)₂, C(N)OH, C(N)OCH⁴, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), cycloSal monophosphate prodrugs, CH₂P(O)(OH)₂, CH₂P(O)(OR⁴)₂, CH₂P(O)(OR⁴)(NR⁴), CH₂P(O)(NR⁴)₂, CH₂P(O)(OH)(OC₁₋₁₀ alkyl-O—C₁₋₂₀ alkyl), and CH₂-cycloSal monophosphate prodrugs,
each R⁴ are independently selected from aryl, heteroaryl, arylalkyl, alkylaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁵, OH, OR⁵, SR⁵, S(O)R⁵, SO₂R⁵, C(O)R⁵, C(O)OR⁵, OC(O)R⁵, OC(O)OR⁵, NH₂, NHR⁵, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, COOH, C(O)H, C(N)NH₂, C(N)NHR⁵, C(N)N(R⁵)₂, C(N)OH, C(N)OCH₃, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
each R⁵ are independently aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁶, OH, OR⁶, SR⁶, S(O)R⁶, SO₂R⁶, C(O)R⁶, C(O)OR⁶, OC(O)R⁶, OC(O)OR⁶, NH₂, NHR⁶, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, COOH, C(O)H, C(N)NH₂, C(N)NHR⁶, C(N)N(R⁶)₂, C(N)OH, C(N)OCH₃, CN, N₃, NO₂, CF₃, CF₂CF₃, OCF₃, OCF₂CF₃, F, Cl, Br, I, P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
each R⁶ are independently aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of C₁₋₁₀ alkyl, C₂₋₁₀ alkenyl, C₂₋₁₀ alkynyl, OH, NH₂, C(O)NH₂, C(O)NHOH, SO₂NH₂, COOH, C(O)H, C(N)NH₂, C(N)OH, C(N)OCH₃, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
or a pharmaceutically-acceptable salt or prodrug thereof, or
or a compound of Formulas (B)—(H): 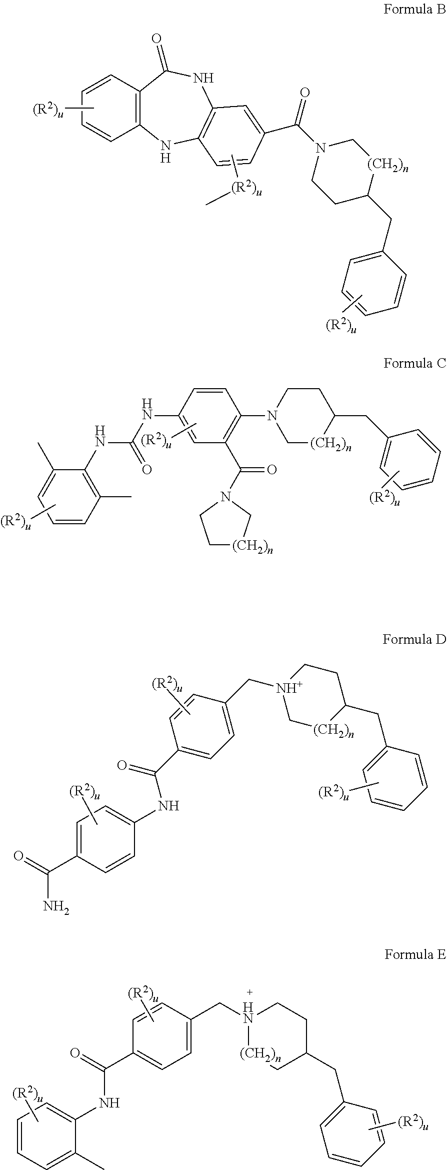 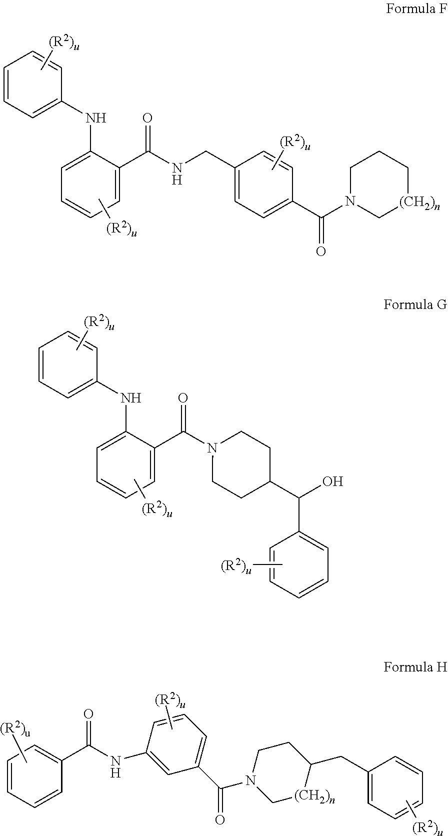
u is, independently 0, 1, 2, 3 or 4;
n is, independently, 0, 1 or 2,
each R² is, independently R³, OH, OR³, SR³, S(O)R³, SO₂R³, C(O)R³, C(O)OR³, OC(O)R³, OC(O)OR³, NH₂, NHR³, NHC(O)R³, NRC(O)R³, NHS(O)₂R³, NR³S(O)₂R³, NHC(O)OR³, NR³C(O)OR³, NHC(O)NH₂, NHC(O)NHR³, NHC(O)N(R³)₂, NR³C(O)N(R³)₂, C(O)NH₂, C(O)NHR³, C(O)N(R³)₂, C(O)NHOH, C(O)NHOR³, C(O)NHSO₂R³, C(O)NR³SO₂R, SO₂NH₂, SO₂NHR³, SO₂N(R³)₂, COOH, C(O)H, C(N)NH₂, C(N)NHR³, C(N)N(R³)₂, C(N)OH, C(N)OCH₃, CN, N₃, NO₂, CF₃, CF₂CF₃, OCF₃, OCF₂CF₃, halo (F, Cl, Br, or I), —CH₂-phosphonate, —CH₂O-phosphate, CH₂P(O)(OH)₂, CH₂P(O)(OR³)₂, CH₂P(O)(OR³)(NR³), CH₂P(O)(NR³)₂, CH₂P(O)(OH)(OC₁₋₁₀ alkyl-O—C₁₋₂₀ alkyl or CH₂-cycloSal monophosphate prodrug,
wherein the term phosphate includes monophosphate, diphosphate, triphosphate, and stabilized phosphate prodrugs, and the term phosphonate includes the same prodrugs that are present in the phosphate prodrugs,
each R³ is, independently, aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁴, OH, OR⁴, SR⁴, S(O)R⁴, SO₂R⁴, C(O)R⁴, C(O)OR⁴, OC(O)R⁴, OC(O)OR⁴, NH₂, NHR⁴, NHC(O)R⁴, NR⁴C(O)R⁴, NHS(O)₂R⁴, NR⁴S(O)₂R⁴, NHC(O)OR⁴, NR⁴C(O)OR⁴, NHC(O)NH₂, NHC(O)NHR⁴, NHC(O)N(R⁴)₂, NR⁴C(O)N(R⁴)₂, C(O)NH₂, C(O)NHR⁴, C(O)N(R⁴)₂, C(O)NHOH, C(O)NHOR⁴, C(O)NHSO₂R⁴, C(O)NR⁴SO₂R⁴, SO₂NH₂, SO₂NHR⁴, SO₂N(R⁴)₂, COOH, C(O)H, C(N)NH₂, C(N)NHR⁴, C(N)N(R⁴)₂, C(N)OH, C(N)OCH⁴, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), cycloSal monophosphate prodrugs, CH₂P(O)(OH)₂, CH₂P(O)(OR⁴)₂, CH₂P(O)(OR⁴)(NR⁴), CH₂P(O)(NR⁴)₂, CH₂P(O)(OH)(OC₁₋₁₀ alkyl-O—C₁₋₂₀ alkyl), and CH₂-cycloSal monophosphate prodrugs,
each R⁴ are independently selected from aryl, heteroaryl, arylalkyl, alkylaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁵, OH, OR₅, SR₅, S(O)R⁵, SO₂R⁵, C(O)R⁵, C(O)OR⁵, OC(O)R⁵, OC(O)OR⁵, NH₂, NHR⁵, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R)₂, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R, SO₂NH₂, SO₂NHR⁵, SO₂N(R⁵)₂, COOH, C(O)H, C(N)NH₂, C(N)NHR⁵, C(N)N(R⁵)₂, C(N)OH, C(N)OCH₃, CN, N₃, NO₂, CF₃, CF₂CF₃, OCF₃, OC₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
each R⁵ are independently aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of R⁶, OH, OR⁶, SR⁶, S(O)R⁶, SO₂R⁶, C(O)R⁶, C(O)OR⁶, OC(O)R⁶, OC(O)OR⁶, NH₂, NHR⁶, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R, SO₂NH₂, SO₂NHR⁶, SO₂N(R⁶)₂, COOH, C(O)H, C(N)NH₂, C(N)NHR⁶, C(N)N(R⁶), C(N)OH, C(N)OCH₃, CN, N₃, NO₂, CF₃, CF₂CF₃, OCF₃, CF₂CF₃, F, Cl, Br, I, P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
each R⁶ are independently aryl, heteroaryl, C₃₋₁₀ cycloalkyl, C₃₋₁₀ cycloalkenyl, heterocycloalkyl, heterocycloalkenyl, C₁₋₁₀ alkyl, C₂₋₁₀ alkenyl or C₂₋₁₀ alkynyl, each of which is unsubstituted or independently substituted with one or more substituents selected from the group consisting of C₁₋₁₀ alkyl, C₂₋₁₀ alkenyl, C₂₋₁₀ alkynyl, OH, NH₂, C(O)NH₂, C(O)NHOH, SO₂NH₂, COOH, C(O)H, C(N)NH₂, C(N)OH, C(N)OCH₃, CN, N₃, NO₂, CF₃, CF₂CF₃, OCF₃, OCF₂CF₃, halo (F, Cl, Br, or I), P(O)(OH)₂, P(O)(OR⁴)₂, P(O)(OR⁴)(NR⁴), P(O)(NR⁴)₂, P(O)(OH)(OC₁₋₁₀ alkyl-O—C₁₋₂₀ alkyl), and cycloSal monophosphate prodrugs,
or a pharmaceutically-acceptable salt or prodrug thereof.

2. The method of claim 1, wherein the compound is a Retinoic Acid Receptor-like Orphan Receptor (ROR) alpha agonist.

3. The method of claim 1, wherein one of X and Z is —C(O)—, —SO₂—, or —NC(O)—, and the other is —NH—, —N(NH₂)—, —N(OH)—, —N(CH₂—O—P(O)(OH)₂)—; —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, or —O—.

4. The method of claim 1, wherein one of X and Z is —C(O)—, —SO₂—, or —N(C(O)—, and the other is —CH₂—, —CH(C₁₋₆ alkyl)-, C(alkyl)₂-, —CH(C₃₋₈ cycloalkyl)-, —CH(C₂₋₆ alkenyl, —CH(C₂₋₆ alkynyl)-, —CH(aryl)-, —CH(heteroaryl)-, —CF₂—, —CCl₂—, —CH(CF₃)—, —CH(OH)—, —CH(OAlkyl)-, —CH(NH₂)—, —CH(NHAlkyl)-, or —CH(C(O)NH₂)—.

5. The compound of claim 1, wherein one of X and Z is —NH—, —N(CH₂—O—P(O)(OH)₂)—; —N(NH₂)—, —N(OH)—, —N(alkyl)-, or —O— and the other is —CH₂—, —CH(C₁₋₆ alkyl)-, C(alkyl)₂-, —CH(C₃₋₈ cycloalkyl)-, —CH(C₂₋₆ alkenyl, —CH(C₂₋₆ alkynyl)-, —CH(aryl)-, —CH(heteroaryl)-, —CF₂—, —CCl₂—, —CH(CF₃)—, —CH(OH)—, —CH(OAlkyl)-, —CH(NH₂)—, —CH(NHAlkyl)-, or —CH(C(O)NH₂)—.

6. The method of claim 1, wherein one of X and Z is —NH—, —N(NH₂)—, —N(CH2-O—P(O)(OH)₂)—; —N(OH)—, —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, and the other is —C(O)— or —SO₂—.

7. The method of claim 1, wherein Y is —NH, —N(NH₂)—, —N(CH₂—O—P(O)(OH)₂)—; —NH(OH)—, —N(C₁₋₁₀ alkyl)-, —N(C₃₋₁₀ cycloalkyl)-, —N(C₂₋₁₀ alkenyl)-, —N(C₂₋₁₀ alkynyl)-, —N(aryl)-, or —N(heteroaryl)-, or —O—.

7. (canceled)

8. The method of claim 1, wherein one of R¹ and R² is H, —CH₂-phosphonate, —CH₂O-phosphate, wherein the term phosphate includes monophosphate, diphosphate, triphosphate, and stabilized phosphate prodrugs, and the term phosphonate includes the same prodrugs that are present in the phosphate prodrugs.

9. The method of claim 1, wherein one of R¹ and R² is H, —CH₂P(O)(OH)₂, —CH₂P(O)(OH)(OR⁶), —CH₂P(O)(OR⁶)₂, —CH₂P(O)(OR⁶)(NR⁶), —CH₂P(O)(NR⁶)₂, —CH₂P(O)(OH)(OC₁₋₁₀ alkyl-O—C₁₋₂₀ alkyl), or a —CH₂-cycloSal monophosphate prodrug.

10. The method of claim 9, wherein one of R¹ and R² is a phosphonate, a phosphoramidate, a cycloSal monophosphate prodrug, or has the formula —CH₂P(O)(OH)(OC₁₋₁₀ alkyl-O—C₁₋₂₀ alkyl).

11. The method of claim 1, wherein one of R¹ and R² is C(O)NHR⁴, C(O)(NR⁴)₂, 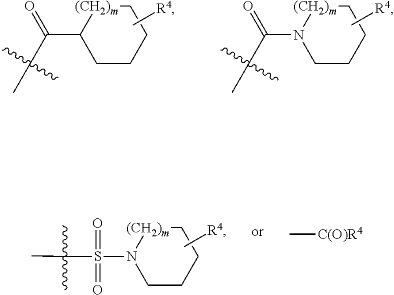
wherein R⁴ is C₁₋₁₀ alkyl, C₃₋₁₀ cycloalkyl, C₂₋₁₀ alkenyl, C₂₋₁₀ alkynyl, C₁₋₁₀ halo alkyl, C₁₋₁₀ alkyl-aryl, or C₁₋₁₀ haloalkyl-aryl and m is 0, 1 or 2.

12. The method of claim 1, wherein one of R¹ and R² is —C(O)—C₁₋₁₀ alkyl, —C(O)-alkylaryl, —C(O)-heterocyclyl-alkylaryl, —C(O)-heterocyclyl-CH₂-aryl, —C(O)— heterocyclyl-CF₂-aryl, —C(O)-cycloalkyl-alkylaryl, —C(O)NHC₁₋₁₀ alkyl, —C(O)NH-alkylaryl, —C(O)NH-heterocyclyl-alkylaryl, —C(O)NH-heterocyclyl-CF₂-aryl, —C(O)NH-cycloalkyl-alkylaryl, —SO₂—C₁₋₁₀ alkyl, —SO₂-alkylaryl, —SO₂-heterocyclyl-alkylaryl, —SO₂-heterocyclyl-CF₂-aryl, or —SO₂-cycloalkyl-alkylaryl.

13. The method of claim 1, wherein the compound has one of the following formulas: 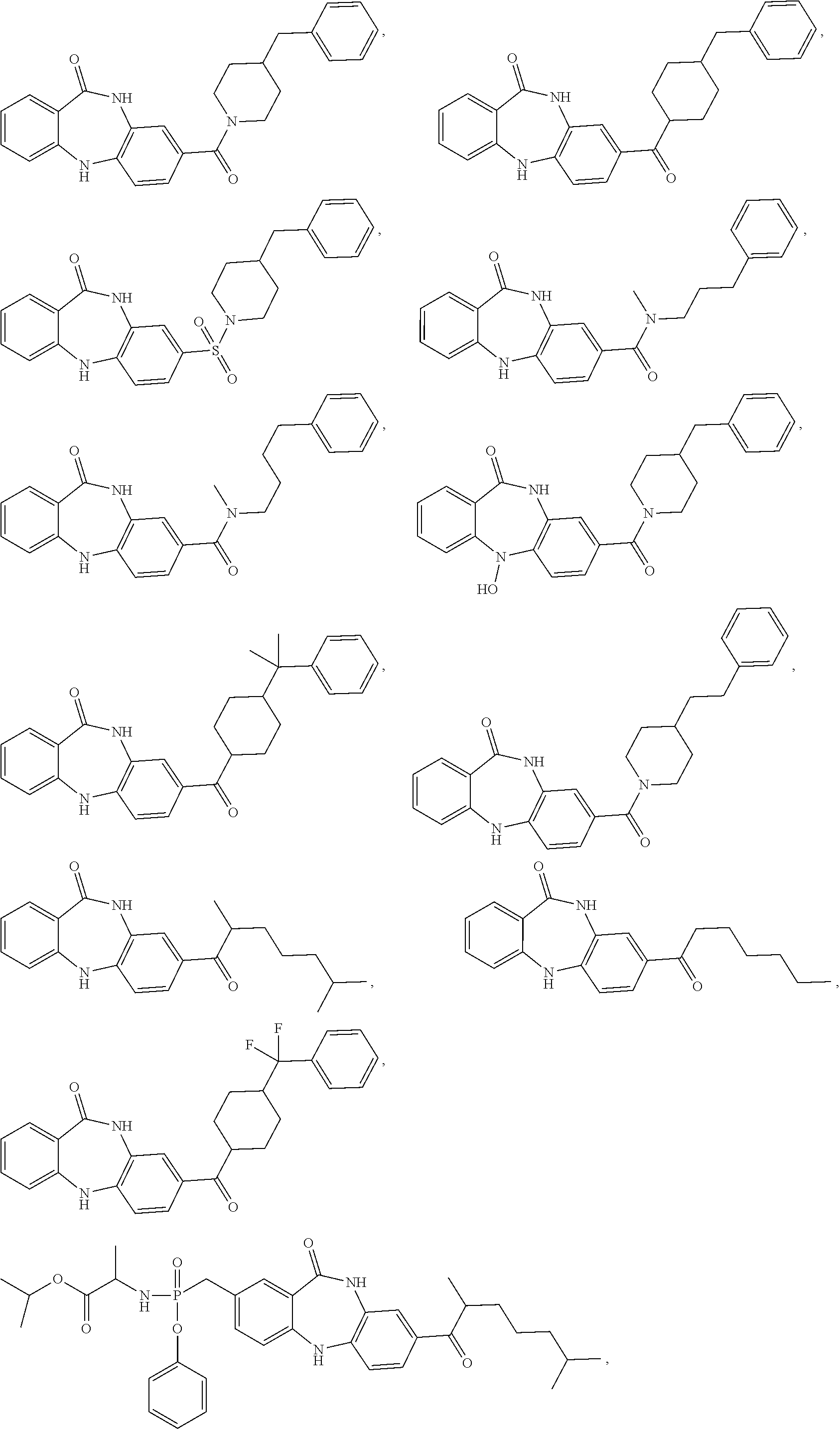 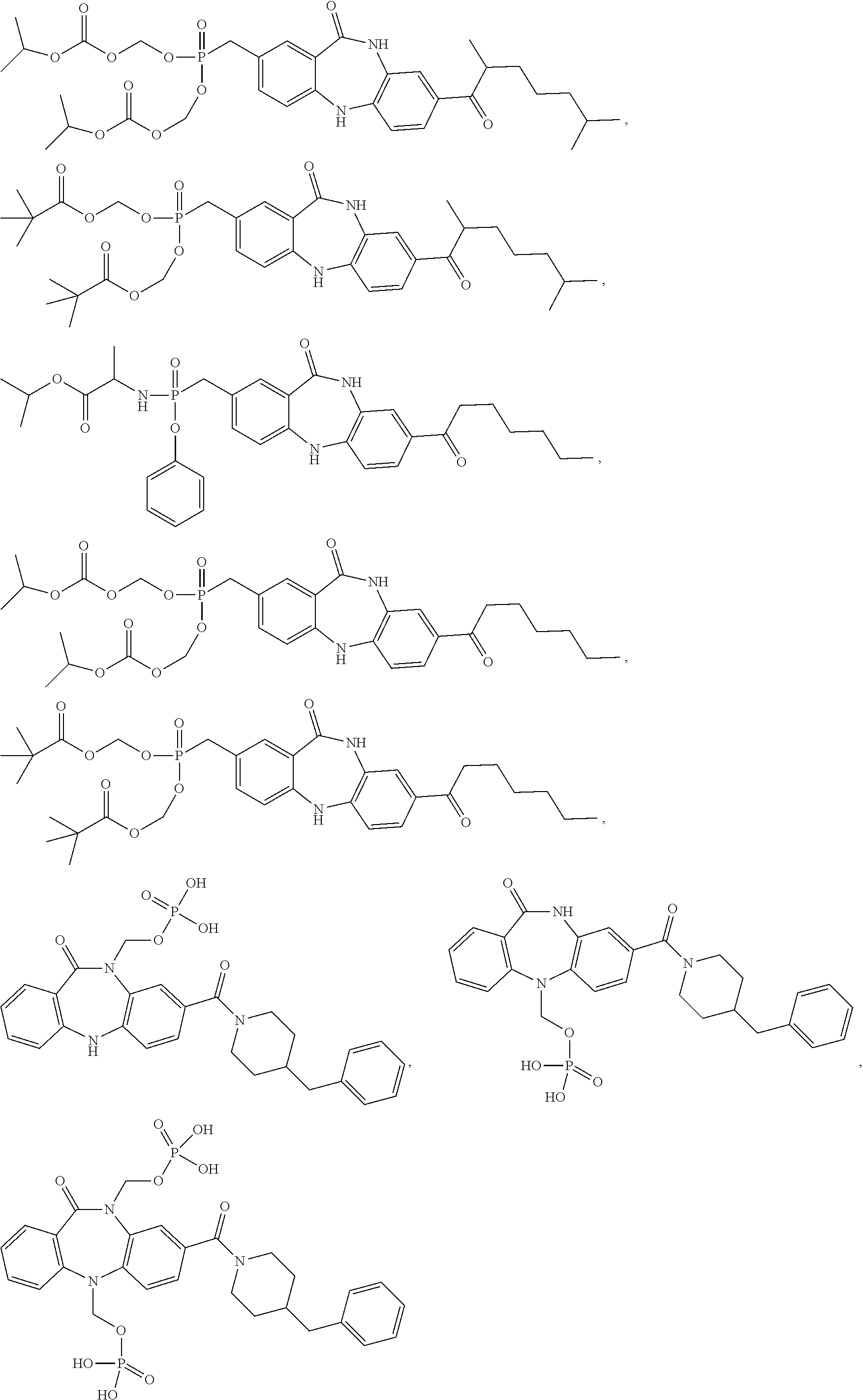
or a pharmaceutically-acceptable salt or prodrug thereof.

14. The method of claim 1, wherein the compound has the formula: 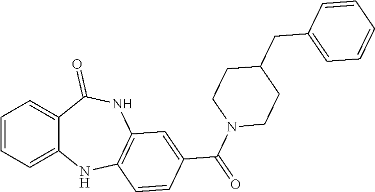 [SMILES: O=C1Nc2cc(C(=O)N3CCC(Cc4ccccc4)CC3)ccc2Nc2ccccc21]
or a pharmaceutically acceptable salt or prodrug thereof.

15. The method of claim 1, wherein the compound is administered in a composition, wherein the composition comprises a pharmaceutically-acceptable carrier or excipient.

16. The method of claim 15, wherein the composition is a transdermal composition or a nanoparticulate composition.

17. The method of claim 1, further comprising administering a second Retinoic Acid Receptor-like Orphan Receptor (ROR) modulator from formula (A).

18. The method of claim 1, further comprising administering one or more additional active agents for treating pancreatitis, sarcopenia, stroke, or traumatic brain injury.

19. The method of claim 15, further comprising administering one or more active agents selected from the group consisting of agents used to treat pancreatitis, sarcopenia, stroke, or traumatic brain injury.

20. The method of claim 1, wherein the pancreatitis is hypertriglyceridemia-induced pancreatitis, pancreatitis caused by Iatrogenic disease (pancreatitis in view of the ERCP procedure), pancreatitis caused by gallstones, or pancreatitis caused by alcohol consumption.

21. The method of claim 1, wherein the compound of Formula (A) is administered prior to, concomitantly with, or following treatments and/or procedures that are associated with an increased risk of pancreatitis.

22. A pharmaceutical composition comprising a compound of claim 1, and one or more active agents selected from the group consisting of statins, ACE inhibitors, oral contraceptives/hormone replacement therapy (HRT), diuretics, antiretroviral therapy, valproic acid, oral hypoglycemic agents; blood thinners, compounds that break up existing blood clots, platelet aggregation inhibitors, anti-coagulants, neuroprotective agents, argatroban, alfimeprase, tenecteplase, ancrod, sildenafil, insulin, insulin growth factor, magnesium sulfate, human serum albumin, caffeinol, microplasmin, a statin, eptifibatide, tinzaparin, enecadin, citicoline, edaravone, cilostazol, Tranexamic acid, sedatives, analgesics, paralytic agents, anti-seizure medications, norepinephrine, insulin, and VLA-1 (Very Late Activation Antigen-I) antagonists, Temozolomide, a cannabinoid, berberine, perillyl alcohol, a radiosensitizer, a boron neutron capture agent, an anticonvulsant, a corticosteroid, chimeric antigen receptor (CAR) T cells using CLTX, IL13Rα2, Her2/CMV, EGFRvIII, CSPG4, NKG2DL, CD19, or CD133 as the targeting domain, MP-Pt(IV), RIPGBM, and Kisquali® (Ribociclib), and combinations thereof.

23-67. (canceled)
